(19) United States
(12) Patent Application Publication
(10) Pub. No.: US 2025/0082643 A1
(43) Pub. Date: Mar. 13, 2025

(54) COMBINATION THERAPY FOR PIK3CA-ASSOCIATED DISEASES OR DISORDERS

(71) Applicant: Faeth Therapeutics, Inc., Austin, TX (US)

(72) Inventors: Oliver D.K. MADDOCKS, Glasgow (GB); Todd YOUNG, San Mateo, CA (US); Marcus GONCALVES, Garden City, NY (US)

(21) Appl. No.: 18/638,481

(22) Filed: Apr. 17, 2024

Related U.S. Application Data
(63) Continuation of application No. PCT/US2022/078418, filed on Oct. 20, 2022.
(60) Provisional application No. 63/270,297, filed on Oct. 21, 2021.

(51) Int. Cl. A61K 31/5377; A23L 33/00; A61K 31/337

(52) U.S. Cl. CPC A61K 31/5377; A23L 33/30; A61K 31/337

(57) ABSTRACT

Disclosed herein are methods of treating a cancer comprising administering an inhibitor of at least one kinase in the insulin-receptor/PI3K/AKT/mTOR pathway. In some embodiments, the kinase inhibitor is administered with an insulin suppressing diet.

### CROSS-REFERENCE

This application is a continuation of International Application No. PCT/2022/078418, filed Oct. 20, 2022 which claims the benefit of U.S. Provisional Application No. 63/270,297, filed Oct. 21, 2021, which is incorporated herein by reference in its entirety.

### BACKGROUND

An association exists between some diseases and disorders and the insulin-receptor phosphatidylinositol kinase (phosphoinositide 3-kinase; PI3K), protein kinase B (AKT), and mammalian target of rapamycin (mTOR) signaling pathway (called the insulin-receptor/PI3K/AKT/mTOR pathway). In cancer, mutations in PIK3CA are observed at similar frequency to mutations in KRAS. Targeting PI3K and upstream and downstream regulators of insulin-receptor/PI3K/AKT/mTOR signaling can be used to treat associated diseases.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, disclosed herein is a method of treating a cancer in a subject in need thereof, the method comprising administering to the subject: a) an insulin suppressing meal; and b) a therapeutically effective amount of serabelisib, wherein the therapeutically effective amount is about 300 mg to about 900 mg.

In some embodiments, disclosed herein is a method of treating a cancer in a subject in need thereof, the method comprising administering to the subject: a) an insulin suppressing meal; b) a therapeutically effective amount of an inhibitor of at least one kinase in the insulin-receptor/PI3K/AKT/mTOR pathway; and c) a therapeutically effective amount of a taxane-based chemotherapeutic agent.

In some embodiments, disclosed herein is a method of treating a cancer in a subject in need thereof, the method comprising administering to the subject: a) an insulin suppressing meal; and b) a therapeutically effective amount of serabelisib, wherein the therapeutically effective amount is about 300 mg to about 900 mg daily for three consecutive days per week, wherein the administration of the serabelisib is oral, wherein the cancer is a solid tumor comprising a PIK3CA mutation.

In some embodiments, disclosed herein is a method of treating a cancer in a subject in need thereof, the method comprising administering to the subject: a) an insulin-suppressing meal; b) a therapeutically effective amount of serabelisib, wherein the therapeutically effective amount is from about 300 mg to about 900 mg; and c) a therapeutically effective amount of nab-paclitaxel, wherein the therapeutically effective amount is about 80 mg/m², wherein the administration of the serabelisib is oral, wherein the administering of the serabelisib is daily for three consecutive days per week, wherein the administration of the nab-paclitaxel is intravenous, wherein the administration of the nab-paclitaxel is once per week.

In some embodiments, disclosed herein is a method of treating a cancer in a subject in need thereof, the method comprising administering to the subject: a) an insulin-suppressing meal; and b) a therapeutically effective amount of a PI3K inhibitor.

In some embodiments, disclosed herein is a method of inhibiting PI3K signaling in a subject in need thereof, the method comprising administering to the subject: a) an insulin-suppressing meal; and b) a therapeutically effective amount of a PI3K inhibitor.

### BRIEF DESCRIPTION OF THE DRAWINGS

FIG. 1 depicts a flowchart of the study using serabelisib in combination with an insulin suppressing diet in subjects with cancer.

FIG. 2 depicts a representation of the study cohorts of the clinical trial using serabelisib in combination with an insulin suppressing diet in subjects with cancer.

FIG. 3 depicts a flowchart of the study using serabelisib in combination with an insulin suppressing diet and nab-paclitaxel in subjects with cancer.

FIG. 4 depicts a representation of the study cohorts of the clinical trial using serabelisib in combination with an insulin suppressing diet with and without nab-paclitaxel in subjects with cancer.

### DETAILED DESCRIPTION

The phosphoinositide 3-kinase (PI3K) signaling pathway is an important regulator of normal cellular processes including cell growth, proliferation, and survival. Abnormalities in this pathway can promote tumor progression and resistance to treatment in a variety of solid tumor cancers. Additionally, PI3K pathway abnormalities can include mutations, often in multiple genes. An association exists between some diseases and disorders and the insulin-receptor PI3K, protein kinase B (AKT), and mammalian target of rapamycin (mTOR) signaling pathway (called the insulin-receptor/PI3K/AKT/mTOR pathway). In cancer, mutations in PIK3CA (PIK3Ca) are observed at similar frequency to mutations in KRAS. Phosphatase and tensin homolog (PTEN) is a tumor suppressor and core inhibitory component of the PI3K signaling pathway. Inherited loss-of-function mutations in the PTEN gene can be associated with more diverse human pathologies, mostly relating to cell and tissue overgrowth and development of many sporadic cancers.

In colorectal cancer, insulin is the best-established biochemical mediator between obesity and colon cancer. Insulin may play a key role in the development of colorectal cancer. An increased concentration of plasma C-peptide (a direct marker of insulin production that is more stable than insulin itself) is significantly associated with an increased risk of colorectal cancer. Additionally, fasting insulin, homeostatic model assessment for insulin resistance (HOMA-IR), glycated hemoglobin (HbA1c), and C-peptide were all significantly associated with increased risk of colorectal cancer. A subset of CRC tumors may have an exaggerated response to serum insulin as activating mutations in PIK3CA are present in 10-20% of colorectal cancers.

Endometrial cancer is associated with clinical biomarkers of hyperinsulinemia such as high fasting insulin, C-peptide (a marker of insulin production), fasting glucose (a marker of insulin resistance), hemoglobin A1c (a direct marker of chronic hyperglycemia), and high dietary glycemic load. Insulin stimulates the growth of the endometrial mucosa, especially in the proliferative phase of the endometrial cycle, during which insulin-like growth factor (IGF) signaling is essential. IGF-1 and insulin bind to the same cell surface tyrosine kinase receptors to activate PI3K to drive the production of macromolecules and proliferation. PI3K activation is sufficient for the initiation of carcinoma in the native adult uterine epithelia, and enhancements in the PI3K signaling pathway dominate the genetics of human endometrial cancer. More than 90% of human endometrial tumors have alterations in the PI3K signaling pathway, most often activating mutations and amplifications in PIK3CA (the gene encoding the catalytic subunit of PI3K), or loss-of-function mutations in PTEN, the gene encoding a phosphatase with activity that directly opposes that of PI3K.

In clear cell carcinoma, the most frequent gene alterations are in the AT-rich interaction domain 1A (ARID1A) and PIK3CA genes (both occurring in about 50% of cases). Additionally, mutations of PTEN are found in 2%-13% of cases. Endometrioid carcinoma accounts for about 10% of all epithelial ovarian cancers, and among the most mutated genes are PIK3CA (43%) and PTEN (30%).

Although therapies targeting the insulin-receptor/PI3K/AKT/mTOR pathway are desirable, the medical community has struggled to identify effective compositions and methods for targeting PI3K as well as upstream and downstream regulators of insulin-receptor/PI3K/AKT/mTOR signaling. Thus, there exists a long-felt and unmet need for compositions and methods for treating a disease or disorder associated with PI3K signaling.

Disclosed herein are compositions and methods for treating a disease or disorder associated with PI3K signaling comprising administering an inhibitor of at least one kinase in the insulin-receptor/PI3K/AKT/mTOR pathway. In some embodiments, the methods disclosed herein further comprise administering a diet that influences the subject's metabolic state, for example, an insulin suppressing meal or diet. In some embodiments, the methods disclosed herein further comprise administering a modulator of glucose metabolism. The disclosure further provides pharmaceutical compositions that can include a pathway inhibitor, modulator of glucose metabolism, or a combination thereof.

Administration of various modulators of glucose metabolism alone or administration of various inhibitors of the insulin-receptor/PI3K/AKT/mTOR pathway can be associated with an unfavorable safety profile and suboptimal anti-cancer activity. However, the types of combination therapies described herein can reduce or eliminate such side effects. For example, the inhibitors may be administered in lower doses or for shorter duration when combined with the other therapeutic agents and/or an insulin suppressing diet. Such combination therapies can provide reduced toxicity and avoid some of the side effects of monotherapies of drug-only therapies.

As disclosed herein, pharmacologic blockade of PI3K elevates serum glucose and raises serum insulin. This hyperinsulinemia can re-activate the PI3K and mTOR signaling pathway in tumors, thereby compromising the effectiveness of PI3K blocking. The present disclosure provides a variety of interventions to reduce serum insulin. In some embodiments, the methods described herein can include administration of serabelisib, a PI3K alpha isoform (PI3Kα) inhibitor, an insulin suppressing diet, or a combination thereof.

TABLE 1 shows human PI3K genes that can be associated with diseases or disorders associated with PI3K signaling.

TABLE 1
PI3K Genes

| Group | Gene | Protein | Aliases |
|---|---|---|---|
| Class 1 | PIK3CA | PI3K, catalytic, alpha polypeptide | p110-α |
| catalytic | PIK3CB | PI3K, catalytic, beta polypeptide | p110-β |
| | PIK3CG | PI3K, catalytic, gamma polypeptide | p110-γ |
| | PIK3CD | PI3K, catalytic, delta polypeptide | p110-δ |
| Class 1 | PIK3R1 | PI3K, regulatory subunit 1 (alpha) | p85-α |
| regulatory | PIK3R2 | PI3K, regulatory subunit 2 (beta) | p85-β |
| | PIK3R3 | PI3K, regulatory subunit 3 (gamma) | p55-γ |
| | PIK3R4 | PI3K, regulatory subunit 4 | p150 |
| | PIK3R5 | PI3K, regulatory subunit 5 | p101 |
| | PIK3R6 | PI3K, regulatory subunit 6 | p87 |
| Class 2 | PIK3C2A | PI3K, class 2, alpha polypeptide | PI3K-C2α |
| | PIK3C2B | PI3K, class 2, beta polypeptide | PI3K-C2β |
| | PIK3C2G | PI3K, class 2, gamma polypeptide | PI3K-C2γ |
| Class 3 | PIK3C3 | PI3K, class 3 | Vps34 |

In some embodiments, disclosed herein is a method of inhibiting PI3K signaling in a subject in need thereof, the method comprising administering to the subject: a) an insulin suppressing meal; and b) a therapeutically effective amount of an inhibitor of at least one kinase in the insulin-receptor/PI3K/AKT/mTOR pathway. In some embodiments, the inhibitor of at least one kinase in the insulin-receptor/PI3K/AKT/mTOR pathway is a PI3K inhibitor. In some embodiments, the inhibitor of at least one kinase in the insulin-receptor/PI3K/AKT/mTOR pathway is a PI3Kα inhibitor, for example, serabelisib.

In some embodiments, disclosed herein is a method of treating a cancer, the method comprising administering to a subject in need thereof: a) an insulin suppressing meal; and b) a therapeutically effective amount of an inhibitor of at least one kinase in the insulin-receptor/PI3K/AKT/mTOR pathway. In some embodiments, the inhibitor of at least one kinase in the insulin-receptor/PI3K/AKT/mTOR pathway is a PI3K inhibitor. In some embodiments, the inhibitor of at least one kinase in the insulin-receptor/PI3K/AKT/mTOR pathway is a PI3Kα inhibitor, for example, serabelisib.

In some embodiments, disclosed herein is a method of treating a cancer in a subject in need thereof, the method comprising administering to the subject: a) an insulin suppressing meal; and b) a therapeutically effective amount of serabelisib, wherein the therapeutically effective amount is about 300 mg to about 900 mg.

In some embodiments, disclosed herein is a method of treating a cancer in a subject in need thereof, the method comprising administering to the subject: a) an insulin suppressing meal; b) a therapeutically effective amount of an inhibitor of at least one kinase in the insulin-receptor/PI3K/AKT/mTOR pathway; and c) a therapeutically effective amount of a taxane-based chemotherapeutic agent.

In some embodiments, disclosed herein is a method of treating a cancer in a subject in need thereof, the method comprising administering to the subject: a) an insulin suppressing meal; and b) a therapeutically effective amount of serabelisib, wherein the therapeutically effective amount is about 300 mg to about 900 mg daily for three consecutive days per week, wherein the administration of the serabelisib is oral, wherein the cancer is a solid tumor comprising a PIK3CA mutation.

In some embodiments, disclosed herein is a method of treating a cancer in a subject in need thereof, the method comprising administering to the subject: a) an insulin-suppressing meal; b) a therapeutically effective amount of serabelisib, wherein the therapeutically effective amount is from about 300 mg to about 900 mg; and c) a therapeutically effective amount of nab-paclitaxel, wherein the therapeutically effective amount is about 80 mg/m², wherein the administration of the serabelisib is oral, wherein the administering of the serabelisib is daily for three consecutive days per week, wherein the administration of the nab-paclitaxel is intravenous, wherein the administration of the nab-paclitaxel is once per week.

In some embodiments, disclosed herein is a method of treating a cancer in a subject in need thereof, the method comprising administering to the subject: a) an insulin-suppressing meal; and b) a therapeutically effective amount of a PI3K inhibitor.

In some embodiments, disclosed herein is a method of inhibiting PI3K signaling in a subject in need thereof, the method comprising administering to the subject: a) an insulin-suppressing meal; and b) a therapeutically effective amount of a PI3K inhibitor.

In some embodiments, the disclosed methods of treating a cancer further comprise administering a standard-of-care (SOC) therapy for the cancer such as those described herein.

### Therapeutic Agents

In some embodiments, a therapeutic agent is administered to a subject in need thereof. In some embodiments, the therapeutic agent is an inhibitor of at least one kinase in the insulin-receptor/PI3K/AKT/mTOR pathway. In some embodiments, the therapeutic agent is a PI3K inhibitor. In some embodiments, the therapeutic agent is a PI3Kα inhibitor. In some embodiments, the therapeutic agent is an AKT inhibitor. In some embodiments, the therapeutic agent is an mTOR inhibitor. In some embodiments, the therapeutic agent is a small molecule. In some embodiments, the therapeutic agent is an antibody. In some embodiments, the therapeutic agent is a protein.

In some embodiments, the inhibitor of at least one kinase in the insulin-receptor/PI3K/AKT/mTOR pathway is serabelisib (TAK-117), idelalisib, copanlisib, buparlisib (BKM120), alpelisib (BYL719), taselisib (GDC-0032), pictilisib (GDC-0941), apitolisib (GDC-0980), dactolisib, MK-2206, linsitinib (OSI-906), or a combination thereof.

A compound described herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

An inhibitor of at least one kinase in the insulin-receptor/PI3K/AKT/mTOR pathway can be administered using a scheduled administration cycle. In some embodiments, the kinase inhibitor of the disclosure can be administered for 1 day, 2 days, 3 days, 4 days, 5 days, 6 days, 7 days, 8 days, 9 days, 10 days, 11 days, or 12 days. In some embodiments, the kinase inhibitor of the disclosure can be administered for 1 day, 2 days, 3 days, 4 days, 5 days, 6 days, 7 days, 8 days, 9 days, 10 days, 11 days, or 12 days over consecutive days. In some embodiments, the kinase inhibitor of the disclosure can be administered for 2 consecutive days. In some embodiments, the kinase inhibitor of the disclosure can be administered for 3 consecutive days. In some embodiments, the kinase inhibitor of the disclosure can be administered for 4 consecutive days. In some embodiments, the kinase inhibitor of the disclosure can be administered for 5 consecutive days.

In some embodiments, administration of a kinase inhibitor of the disclosure is followed by 1 day, 2 days, 3 days, 4 days, 5 days, 6 days, 7 days, 8 days, 9 days, 10 days, 11 days, or 12 days without administration of the kinase inhibitor. In some embodiments, administration of a kinase inhibitor of the disclosure is followed by 1 day, 2 days, 3 days, 4 days, 5 days, 6 days, 7 days, 8 days, 9 days, 10 days, 11 days, or 12 days consecutively without administration of the kinase inhibitor. In some embodiments, administration of a kinase inhibitor of the disclosure is followed by 3 consecutive days without administration of the kinase inhibitor. In some embodiments, administration of a kinase inhibitor of the disclosure is followed by 4 consecutive days without administration of the kinase inhibitor. In some embodiments, administration of a kinase inhibitor of the disclosure is followed by 5 consecutive days without administration of the kinase inhibitor.

In some embodiments, the kinase inhibitor of the disclosure can be administered for a first period of time, followed by no administration of the kinase inhibitor for a second period of time. In some embodiments, the kinase inhibitor of the disclosure can be administered for 1 day, followed by 6 consecutive days without administration of the kinase inhibitor. In some embodiments, the kinase inhibitor of the disclosure can be administered for 2 days, followed by 5 consecutive days without administration of the kinase inhibitor. In some embodiments, the kinase inhibitor of the disclosure can be administered for 3 days, followed by 4 consecutive days without administration of the kinase inhibitor. In some embodiments, the kinase inhibitor of the disclosure can be administered for 4 days, followed by 3 consecutive days without administration of the kinase inhibitor. In some embodiments, the kinase inhibitor of the disclosure can be administered for 5 days, followed by 2 consecutive days without administration of the kinase inhibitor. In some embodiments, the kinase inhibitor of the disclosure can be administered for 6 days, followed by 1 day without administration of the kinase inhibitor.

In some embodiments, the periods of administering the kinase inhibitor of the disclosure and periods without administration of the kinase inhibitor are cycled over a period of 1 week, 2 weeks, 3 weeks, 4 weeks, 5 weeks, 6 weeks, 7 weeks, 8 weeks, 9 weeks, 10 weeks, 11 weeks, or 12 weeks. In some embodiments, the periods of administering the kinase inhibitor of the disclosure and periods without administration of the kinase inhibitor are cycled over a period of 3 weeks. In some embodiments, the periods of administering the kinase inhibitor of the disclosure and periods without administration of the kinase inhibitor are cycled over a period of 4 weeks. In some embodiments, the periods of administering the kinase inhibitor of the disclosure and periods without administration of the kinase inhibitor are cycled over a period of 5 weeks. In some embodiments, the periods of administering the kinase inhibitor of the disclosure and periods without administration of the kinase inhibitor are cycled over a period of 6 weeks.

In some embodiments, the inhibitor of at least one kinase in the insulin-receptor/PI3K/AKT/mTOR pathway is serabelisib. Serabelisib (6-(2-Aminobenzo[d]oxazol-5-yl) imidazo[1,2-a]pyridine-3-yl morpholino methanone) is a potent and selective small molecule inhibitor of PI3Kα, with a half maximal inhibitory concentration (IC⁵⁰) of 20 nM in biochemical assays, and >50-fold selectivity over other class I PI3K isoforms and the mammalian target of rapamycin (mTOR). Serabelisib blocks downstream signaling of PI3K in cell lines with hyperactivation of the pathway and demonstrates antiproliferative activity across a range of cell lines. Serabelisib shows strong tumor growth inhibition in mouse xenograft models of human breast cancer cells harboring mutations in the PIK3CA gene but has no efficacy on a renal cell carcinoma xenograft with mutations in the PTEN gene. PTEN null mutations can increase sensitivity to insulin. Thus, insulin suppressing diets can restore sensitivity to PI3K inhibition.

In some embodiments, serabelisib can be administered in an amount of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg. In some embodiments, serabelisib can be administered in an amount of from 300 mg to about 900 mg. In some embodiments, serabelisib can be administered in an amount of from 200 mg to about 500 mg. In some embodiments, serabelisib can be administered in an amount of from about 500 mg to about 750 mg. In some embodiments, serabelisib can be administered in an amount of from about 750 mg to about 1000 mg.

In some embodiments, serabelisib can be administered in an amount of about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg. In some embodiments, serabelisib can be administered in an amount of about 300 mg. In some embodiments, serabelisib can be administered in an amount of about 600 mg. In some embodiments, serabelisib can be administered in an amount of about 900 mg.

In some embodiments, serabelisib can be administered 1, 2, 3, 4, 5, or 6 times a day. In some embodiments, serabelisib can be administered once a day. In some embodiments, serabelisib can be administered twice a day. In some embodiments, serabelisib can be administered three times a day.

The starting dose of serabelisib can be 150, 200, 250, 300, 350, 400, 450, 500, 550, 600, 650, 700, 750, 800, 850, 900, 950, or 1000 mg once daily. Dosing can increase, decrease, remain constant, or a combination thereof throughout the course of treatment. Dosing can be modified (e.g., increased or decreased) by 5, 10, 15, 20, 30, 40, 50, 60, 70, 80, 90, 100, 200, 300, 400, 500, 600, 700, 800, 900, or 1000 mg.

In some embodiments, administration of serabelisib is oral.

Serabelisib exhibits consistent pharmacokinetics by oral administration in various animal species, with rapid absorption and high bioavailable. Following oral dosing, the radiolabeled drug is well distributed to all tissues. The in vitro metabolism of serabelisib is similar across test species and no unique human metabolite was identified. The majority of the drug is excreted in urine and feces as the parent compound.

Radiolabeled serabelisib is metabolized via a number of pathways including oxidation, hydroxylation, dehydrogenation, and to a minor extent by glucuronidation, sulfation, and reduced glutathione conjugation. Multiple human cytochrome P450 (CYP) enzymes (CYP3A4/5, 1A2, 2C8, and 2C9) may contribute to serabelisib metabolism, with CYP3A4 responsible for 72% of the metabolism when normalized for liver content.

Serabelisib displays low potential for inhibition of CYP3A4/5, CYP2D6, CYP2C19, CYP2C9, CYP2C8, CYP2B6, and CYP1A2. Serabelisib does not induce CYP1A2, 2B6, or 3A4/5 activity. The potential for serabelisib to cause exposure changes to concomitantly administered CYP substrates is low. However, there is a potential for co-administered CYP3A4 inhibitors and inducers to affect the exposure of serabelisib. Because serabelisib is an inhibitor of organic cation transporter (OCT) 1, OCT2, and breast cancer resistance protein (BCRP) transporting proteins, serabelisib may affect the exposures of co-administered OCT1, OCT2, and BCRP substrates.

In some embodiments, the inhibitor of at least one kinase in the insulin-receptor/PI3K/AKT/mTOR pathway is idelalisib. Idelalisib is a PI3Kδ inhibitor. By inhibiting PI3Kδ expressed in normal and malignant B-cells, idelalisib induces apoptosis and prevents proliferation in cell lines derived from malignant B-cells and in primary tumor cells. Idelalisib can be used for the treatment of hematological malignancies.

In some embodiments, the inhibitor of at least one kinase in the insulin-receptor/PI3K/AKT/mTOR pathway is copanlisib. Copanlisib is a PI3K inhibitor with inhibitory activity predominantly against the PI3Kα and PI3Kδ isoforms expressed in malignant B-cells. Copanlisib can be used for the treatment of follicular lymphoma.

In some embodiments, the inhibitor of at least one kinase in the insulin-receptor/PI3K/AKT/mTOR pathway is buparlisib. Buparlisib is a pan-class I PI3K inhibitor. Buparlisib can be used for the treatment of breast cancer and head and neck squamous cell carcinoma.

In some embodiments, the inhibitor of at least one kinase in the insulin-receptor/PI3K/AKT/mTOR pathway is alpelisib. Alpelisib selectively inhibits class I PI3K p110α, the catalytic subunit of PI3K. Alpelisib can be used for the treatment of metastatic breast cancer. In some cases, alpelisib is administered in combination with fulvestrant.

In some embodiments, the inhibitor of at least one kinase in the insulin-receptor/PI3K/AKT/mTOR pathway is taselisib. Taselisib is a PI3K inhibitor with inhibitory activity against the PI3Kα/δ/γ isoforms. Taselisib has a dual mechanism of action, both blocking kinase signaling and inducing downregulation of the mutant p110α. Taselisib can be used for the treatment of metastatic breast cancer. In some cases, taselisib is administered in combination with fulvestrant.

In some embodiments, the inhibitor of at least one kinase in the insulin-receptor/PI3K/AKT/mTOR pathway is pictilisib. Pictilisib is a PI3Kα/δ selective inhibitor. Pictilisib can be used for the treatment of advanced breast cancer. In some cases, taselisib is administered in combination with paclitaxel, bevacizumab or trastuzumab, and/or letrozole.

In some embodiments, the inhibitor of at least one kinase in the insulin-receptor/PI3K/AKT/mTOR pathway is apitolisib. Apitolisib is a dual inhibitor of PI3K and mTOR with inhibitory activity against the PI3Kα/β/δ/γ isoforms. Apitolisib can be used for the treatment of hematological malignancies.

In some embodiments, the inhibitor of at least one kinase in the insulin-receptor/PI3K/AKT/mTOR pathway is dactolisib. Dactolisib is a dual inhibitor of PI3K and mTOR with inhibitory activity against the PI3Kα/β/δ/γ isoforms. Dactolisib can be used for the treatment of glioblastomas.

In some embodiments, the inhibitor of at least one kinase in the insulin-receptor/PI3K/AKT/mTOR pathway is MK-2206. MK-2206 is an AKT inhibitor. MK-2206 can be used for the treatment of breast cancer and endometrial cancer.

In some embodiments, the inhibitor of at least one kinase in the insulin-receptor/PI3K/AKT/mTOR pathway is linsitinib. Linsitinib is an inhibitor of IGF-IR and the insulin receptor. Linsitinib can be used for the treatment of adrenocortical carcinoma.

In some cases, the method includes co-administering a pathway inhibitor (such as an inhibitor of at least one kinase in the insulin-receptor PI3K/AKT/mTOR pathway) and a modulator of glucose metabolism. In some embodiments, the modulator of glucose metabolism is a glucose-uptake inhibitor. In some embodiments, the glucose-uptake inhibitor is a sodium-glucose-linked transport protein 1 (SGLT1). In some embodiments, the glucose-uptake inhibitor is a sodium-glucose-linked transport protein 2 (SGLT2). In some embodiments, the glucose-uptake inhibitor is a dual SGLT1/SGLT2 inhibitor. In some embodiments, the methods of the disclosure comprise administering one glucose metabolism modulator. In some embodiments, the methods of the disclosure comprise administering two glucose metabolism modulators. In some embodiments, the methods of the disclosure comprise administering three glucose metabolism modulators.

In some embodiments, the modulator of glucose metabolism is metformin, dapagliflozin, empagliflozin, canagliflozin, ipragliflozin, tofogliflozin, sergliflozin etabonate, remogliflozin etabonate, ertugliflozin, sotagliflozin, conagliflozin, or a combination thereof. In some embodiments, the modulator of glucose metabolism is metformin or a pharmaceutically acceptable salt thereof. In some embodiments the methods of the disclosure further comprise administering about 500 mg of metformin twice a day. In some embodiments the methods of the disclosure further comprise administering about 800 mg of metformin once a day. In some embodiments, the methods of the disclosure further comprise administering less than about 500 mg of metformin twice a day. In some embodiments the methods of the disclosure further comprise administering less than about 800 mg of metformin once a day.

PI3Kα is involved in maintaining glucose homeostasis following activation by insulin receptor signaling. Consequently, a pharmacological effect of targeted PI3Kα inhibition is hyperglycemia. Hyperglycemia induces hyperinsulinemia as the pancreas releases insulin in an attempt to regulate glycemia. In PI3K inhibitors with alpha specificity, hyperglycemia is a frequent adverse event (AE) that reduces tolerability. Hyperglycemia occurs more in the elderly (>75 years old) and those who were diabetic or prediabetic at the start of treatment. PI3K-induced hyperglycemia is commonly managed with diabetic medications such as metformin, insulin secretagogues, or exogenous insulin. In addition to reducing tolerability, hyperglycemia may reduce the efficacy of PI3K inhibitors. The profound hyperinsulinemia that follows PI3K inhibition can activate insulin receptors on cancer cells and reactivate PI3Kα in the tumor.

In some embodiments, the methods of the disclosure further comprise administering a therapeutically effective amount of an antihyperglycemia agent. In some embodiments, the antihyperglycemia agent is metformin, acarbose, a sodium-glucose cotransporter-2 inhibitor (SGLT2i), a dipeptidyl peptidase-4 (DPP-4) inhibitor, a thiazolidinedione, a GLP-1 receptor agonist, an α-glucosidase inhibitor, a meglitinide, a sulfonylurea, or insulin. In some embodiments, the DPP-4 inhibitor is alogliptin, saxagliptin, linagliptin, or sitagliptin. In some embodiments, the thiazolidinedione is pioglitazone or rosiglitazone. In some embodiments, the GLP-1 receptor agonist is exenatide, dulaglutide, semaglutide, or liraglutide. In some embodiments, the α-glucosidase inhibitor is acarbose. In some embodiments, the meglitinide is nateglinide or repaglinide. In some embodiments, the sulfonylurea is glimepiride, glipizide, glipizide xr, glyburide*, or glyburide micronized.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

Inhibition of the PI3K/AKT/mTOR pathway can reduce cancer resistance to taxanes or toxoids. Thus, inhibition of that pathway can re-sensitize tumors to the effects of paclitaxel. In some embodiments, the methods of the disclosure further comprise administering a therapeutically effective amount of a taxane-based chemotherapeutic agent. In some embodiments, the taxane-based chemotherapeutic agent is paclitaxel, nab-paclitaxel, docetaxel, or cabazitaxel.

In some embodiments, the taxane-based chemotherapeutic agent is nab-paclitaxel. Nab-paclitaxel is a form of paclitaxel that is bound to the human protein albumin and contained in nanoparticles. This albumin-bound, solvent-free formulation of paclitaxel does not require steroid premedication and has an improved therapeutic index compared to paclitaxel.

In some embodiments, nab-paclitaxel is administered in an amount of from about 50 mg/m² to about 300 mg/m², 50 mg/m² to about 100 mg/m², 100 mg/m² to about 200 mg/m², or 200 mg/m² to about 300 mg/m². In some embodiments, nab-paclitaxel can be administered in an amount of about 50 mg/m², about 60 mg/m², about 70 mg/m², about 75 mg/m², about 80 mg/m², about 90 mg/m², about 100 mg/m², about 110 mg/m², about 120 mg/m², about 125 mg/m², about 130 mg/m², about 140 mg/m², about 150 mg/m², about 160 mg/m², about 170 mg/m², about 180 mg/m², about 190 mg/m², about 200 mg/m², about 210 mg/m², about 220 mg/m², about 230 mg/m², about 240 mg/m², about 250 mg/m², about 260 mg/m², about 270 mg/m², about 280 mg/m², about 290 mg/m², or about 300 mg/m².

In some embodiments, nab-paclitaxel is administered once weekly. In some embodiments, nab-paclitaxel is administered once weekly for three weeks. In some embodiments, nab-paclitaxel is administered once weekly for three weeks over 4 weeks (3-weeks-on/1-week-off cycle).

In some embodiments, administration of nab-paclitaxel is intravenous.

In some embodiments, a dose of nab-paclitaxel is intravenously administered for about 10-60 minutes, about 10-50 minutes, about 10-40 minutes, about 10-30 minutes, about 10-20 minutes, about 20-30 minutes, about 20-40 minutes, about 20-50 minutes, about 20-60 minutes, about 30-40 minutes, about 30-50 minutes, about 30-60 minutes, about 40-50 minutes, about 40-60 minutes, or about 50-60 minutes. In some embodiments, a dose of nab-paclitaxel is intravenously administered for about 10 minutes, about 20 minutes, about 30 minutes, about 40 minutes, about 50 minutes, or about 60 minutes. In some embodiments, a dose of nab-paclitaxel is intravenously administered for about 30 minutes.

In some embodiments, a dose of nab-paclitaxel is intravenously administered for about 30 minutes once weekly. In some embodiments, a dose of nab-paclitaxel is intravenously administered for about 30 minutes every 3 weeks.

For metastatic breast cancer (MBC), a recommended dosage of nab-paclitaxel is 260 mg/m² intravenously over 30 minutes every 3 weeks. For non-small cell lung cancer (NSCLC), a recommended dosage of nab-paclitaxel is 100 mg/m² intravenously over 30 minutes on Days 1, 8, and 15 of each 21-day cycle. In some cases, carboplatin is further administered on Day 1 of each 21-day cycle immediately after nab-paclitaxel administration.

For adenocarcinoma of the pancreas, a recommended dosage of nab-paclitaxel is 125 mg/m² intravenously over 30-40 minutes on Days 1, 8, and 15 of each 28-day cycle. In some cases, gemcitabine is further administered on Days 1, 8, and 15 of each 28-day cycle immediately after nab-paclitaxel administration.

The methods of the disclosure further comprise administering a therapeutically effective amount of one or more chemotherapeutic agents. In some embodiments, a method can comprise administering to a subject: a) an insulin suppressing meal; b) a therapeutically effective amount of an inhibitor of at least one kinase in the insulin-receptor/PI3K/AKT/mTOR pathway; and c) a therapeutically effective amount of a chemotherapeutic agent. In other embodiments, a method can comprise administering to a subject: a) an insulin suppressing meal; b) a therapeutically effective amount of an inhibitor of at least one kinase in the insulin-receptor/PI3K/AKT/mTOR pathway; c) a therapeutically effective amount of a taxane-based chemotherapeutic agent; and d) a therapeutically effective amount of a chemotherapeutic agent.

The chemotherapeutic agent can include one or more of the following categories of anti-cancer agents:
(i) antiproliferative/antineoplastic drugs and combinations thereof, such as alkylating agents (e.g., cisplatin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, carmustine, lomustine, and dacarbazine); antimetabolites (e.g., gemcitabine, antifolates, such as fluoropyrimidines (5-FU and tegafur), raltitrexed, methotrexate, pemetrexed, cytosine arabinoside, floxuridine, cytarabine, 6-mercaptopurine, 6-thioguanine, fludarabine phosphate, and hydroxyurea); antibiotics (e.g., anthracyclines such as adriamycin, bleomycin, doxorubicin, daunomycin, epirubicin, idarubicin, mitomycin-C, dactinomycin, and mithramycin); antimitotic agents (e.g., vinca alkaloids such as vincristine, vinblastine, vindesine, and vinorelbine; taxanes/taxoids such as paclitaxel, nab-paclitaxel, docetaxel, and cabazitaxel; and polokinase inhibitors); proteasome inhibitors, e.g., carfilzomib and bortezomib; interferon therapy; and topoisomerase inhibitors (e.g., epipodophyllotoxins such as etoposide and teniposide, amsacrine, topotecan, irinotecan, mitoxantrone, and camptothecin); bleomcin, dactinomycin, daunorubicin, doxorubicin, epirubicin, idarubicin, ara-C, paclitaxel (Taxol™), nab-paclitaxel, docetaxel, mithramycin, deoxycoformycin, mitomycin-C, L-asparaginase, interferons (e.g., IFN-alpha), etoposide, teniposide, DNA-demethylating agents, (e.g., azacitidine or decitabine); and histone de-acetylase (HDAC) inhibitors (e.g., vorinostat, MS-275, panobinostat, romidepsin, valproic acid, mocetinostat (MGCD0103), and pracinostat SB939);
(ii) cytostatic agents such as antiestrogens (e.g., tamoxifen, fulvestrant, toremifene, raloxifene, droloxifene and iodoxyfene), antiandrogens (e.g., bicalutamide, flutamide, nilutamide and cyproterone acetate), LHRH antagonists or LHRH agonists (e.g., goserelin, leuprorelin, and buserelin), progestogens (e.g., megestrol acetate), aromatase inhibitors (e.g., as anastrozole, letrozole, vorazole and exemestane) and inhibitors of 5α-reductase such as finasteride; and navelbene, CPT-II, anastrazole, letrazole, capecitabine, reloxafme, cyclophosphamide, ifosamide, and droloxafine;
(iii) anti-invasion agents, e.g., dasatinib and bosutinib (SKI-606), and metalloproteinase inhibitors, inhibitors of urokinase plasminogen activator receptor function or antibodies to heparanase;
(iv) inhibitors of growth factor function: e.g., such inhibitors include growth factor antibodies and growth factor receptor antibodies, e.g., the anti-erbB2 antibody trastuzumab, the anti-EGFR antibody panitumumab, the anti-erbB1 antibody cetuximab, tyrosine kinase inhibitors, e.g., inhibitors of the epidermal growth factor family (e.g., EGFR family tyrosine kinase inhibitors such as gefitinib, erlotinib, canertinib (CI-1033), afatinib, vandetanib, osimertinib, rociletinib, and lapatinib), and antibodies to costimulatory molecules such as CTLA-4, 4-IBB and PD-I, or antibodies to cytokines (IL-10, TGF-beta); inhibitors of the hepatocyte growth factor family; inhibitors of the insulin growth factor family; modulators of protein regulators of cell apoptosis (e.g., Bcl-2 inhibitors); inhibitors of the platelet-derived growth factor family such as imatinib and/or nilotinib (AMN107); inhibitors of serine/threonine kinases (e.g., Ras/Raf signaling inhibitors such as farnesyl transferase inhibitors, sorafenib, tipifarnib, and lonafarnib), inhibitors of cell signaling through MEK and/or AKT kinases, c-kit inhibitors, Abl kinase inhibitors, PI3K inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; CCR2, CCR4, or CCR6 antagonists; and RAF kinase inhibitors.
(v) antiangiogenic agents such as those that inhibit the effects of VEGF, e.g., the anti-VEGF antibody bevacizumab; thalidomide; lenalidomide; and e.g., a VEGF receptor tyrosine kinase inhibitor such as vandetanib, vatalanib, sunitinib, axitinib, and pazopanib;
(vi) gene therapy approaches, including, e.g., approaches to replace aberrant genes such as aberrant p53 or aberrant BRCA1 or BRCA2;
(vii) immunotherapy approaches, including e.g., antibody therapy such as alemtuzumab, rituximab, ibritumomab tiuxetan, and ofatumumab; interferons such as interferon α; interleukins such as IL-2 (aldesleukin); interleukin inhibitors e.g., IRAK4 inhibitors; cancer vaccines including prophylactic and treatment vaccines such as HPV vaccines, e.g., Gardasil®, Cervarix®, Oncophage®, and sipuleucel-T (Provenge®); gp100; dendritic cell-based vaccines (such as Ad.p53 DC); toll-like receptor modulators e.g., TLR-7 or TLR-9 agonists; PD-1, PD-L1, PD-L2, and CTL4-A modulators (e.g., nivolumab), antibodies and vaccines; indoleamine 2,3-dioxygenase 1 (IDO1) inhibitors (such as indoximod); anti-PD-1 monoclonal antibodies (such as MK-3475 and nivolumab); anti-PDL1 monoclonal antibodies (such as MEDI-4736 and RG-7446); anti-PDL2 monoclonal antibodies; and anti-CTLA-4 antibodies (such as ipilumumab);
(viii) cytotoxic agents e.g., fludaribine (fludara), cladribine, and pentostatin;
(ix) targeted therapies, e.g., PI3K inhibitors, e.g., idelalisib and perifosine; SMAC (second mitochondria-derived activator of caspases) mimetics, also known as inhibitor of apoptosis proteins (IAP) antagonists. These agents act to suppress IAPs, e.g., XIAP, cIAP1, and cIAP2, and thereby re-establish cellular apoptotic pathways. Particular SMAC mimetics include birinapant (TL32711), LCL161 (Novartis), AEG 40730, SM-164, LBW242, ML101, AT-406, GDC-0917, AEG 35156, and HGS1029; and agents which target ubiquitin proteasome system (UPS), e.g., bortezomib, carfilzomib, marizomib (NPI-0052), and MLN9708; and
(xii) chimeric antigen receptors, anticancer vaccines, and arginase inhibitors.

The therapeutic agents used in the methods of the disclosure can be a single agent or a combination of agents. In some embodiments, such combinations include agents that have different mechanisms of action. Such combinations can be administered simultaneously, separately, or sequentially. In some embodiments, a combination therapy provided herein is to simultaneously administered. In some embodiments, a combination therapy provided herein is to separately administered. In some embodiments, a combination therapy provided herein is to sequentially administered.

In some embodiments, the methods of the disclosure further comprise administering a therapeutically effective amount of a radiotherapy. The radiotherapy can be administered simultaneously, separately, or sequentially with one or more compositions described herein. In some embodiments, a subject can be treated with a composition of the disclosure and radiotherapy, then go off treatment before beginning a subsequent treatment cycle with the composition and radiotherapy. In some embodiments, the length of the treatment period and off-treatment period are identical. In some embodiments, the length of the treatment period and off-treatment period are different. In some embodiments, the length of the treatment period is longer than the off-treatment period. In some embodiments, the length of the treatment period is shorter than the off-treatment period. Radiotherapy treatments can include, e.g., X-ray therapy, proton beam therapy or electron beam therapies.

### Pharmaceutically-Acceptable Salts

The present disclosure provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base comprises or consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Pharmaceutical Compositions

A pharmaceutical composition of the disclosure can be used, for example, before, during, or after treatment of a subject with, for example, another pharmaceutical agent. A pharmaceutical composition of the disclosure can be used, for example, before, during, or after treatment of a subject with an insulin suppressing meal or diet, such as those described herein.

Subjects can be, for example, elderly adults, adults, adolescents, pre-adolescents, children, toddlers, infants, neonates, and non-human animals. In some embodiments, a subject is a patient. In some embodiments, a subject is ≥18 years of age.

In some embodiments, a subject has a cancer. In some embodiments, a subject has a solid tumor. In some embodiments, a subject has an adenocarcinoma of the colon or rectum. In some embodiments, a subject has an endometrial adenocarcinoma, e.g., endometrioid adenocarcinoma, serous adenocarcinoma, undifferentiated carcinoma, clear cell adenocarcinoma, mixed epithelial carcinoma, adenocarcinoma not otherwise specified (N.O.S.), mucinous adenocarcinoma, squamous cell carcinoma, transitional cell carcinoma, or carcinosarcoma. In some embodiments, a subject has an ovarian clear cell or endometrioid carcinoma. In some embodiments, a subject has a cancer harboring a PIK3CA-activating mutation with or without a PTEN loss-of-function mutation.

In some embodiments, the subject failed, was intolerant of, or ineligible for no more than 3 prior lines of systemic therapy (LOT) for advanced/metastatic disease or refused standard-of-care therapy (excluding neoadjuvant/adjuvant therapy). In some embodiments, the subject failed, was intolerant of, ineligible for, or has refused standard-of-care therapy for advanced/metastatic disease (excluding neoadjuvant/adjuvant therapy).

In some embodiments, the subject has a life expectancy of at least 3 months.

In some embodiments, the subject is not pregnant or suspected of being pregnant. In some embodiments, the subject does not have a primary brain tumor. In some embodiments, the subject does not have Leptomeningeal disease and symptomatic or untreated brain metastases. In some embodiments, the subject does not have diabetes. In some embodiments, the subject does not have diabetes requiring insulin therapy, e.g., type 1 diabetes. In some embodiments, the subject is not undergoing a therapy comprising a CYP3A4 inducer or a CYP3A4 inhibitor. In some embodiments, the subject is not undergoing a specialized diet or a weight loss plan.

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino) propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution, or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers, and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16, or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins 1999), each of which is incorporated by reference in its entirety.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV).

Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

The disclosure further relates to pharmaceutical compositions comprising a pathway inhibitor and modulator of glucose metabolism.

Compositions of the disclosure can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Insulin Suppressing Diet and Dietary Supplements

In some embodiments, a subject is administered a special meal with a PI3K inhibitor of the disclosure. The meal can be an insulin suppressing meal. The insulin suppressing diet can comprise one or more insulin suppressing meals, e.g., breakfast, lunch, snacks, and/or dinner. The insulin suppressing diet can comprise one, two, three, or more insulin suppressing meals daily. In some embodiments, the insulin suppressing diet comprises three meals daily, for example, breakfast, lunch, and dinner.

In some embodiments, a subject is administered a special diet with a PI3K inhibitor of the disclosure. In some embodiments, the diet is a whole food diet. In some embodiments, the diet is an insulin suppressing diet. In some embodiments, the diet is a ketogenic diet. An insulin suppressing diet suppresses insulin production and enhances fat oxidation, which both promote a loss in total body adiposity. In the context of a ‘hypocaloric’ insulin suppressing diet, the body derives most of its energy from fat, but the source of fat is from a mix of dietary ‘exogenous’ fat and endogenous fat mobilized from adipose tissue. On the other hand, exogenous fat is a major source of ketones and fuel during a ‘eucaloric’ insulin suppressing diet.

An insulin suppressing diet can avoid foods that have high levels of sugar or carbohydrates. In some embodiments, an insulin suppressing diet can comprise from about 0.1 g to about 10 g, from about 1 g to about 10 g, from about 10 g to about 20 g, from about 20 g to about 20 g, from about 30 g to about 40 g, from about 40 g to about 50 g, or from about 50 g to about 60 g of carbohydrates per day. In some embodiments, an insulin suppressing diet can comprise from about 0.1 g to about 9 g of carbohydrates per day. In some embodiments, an insulin suppressing diet can comprise from about 1 g to about 9 g of carbohydrates per day. In some embodiments, an insulin suppressing diet can comprise from about 20 g to about 30 g of carbohydrates per day. In some embodiments, an insulin suppressing diet can comprise from about 30 g to about 40 g of carbohydrates per day. In some embodiments, an insulin suppressing diet can comprise from about 40 g to about 50 g of carbohydrates per day.

In some embodiments, an insulin suppressing diet can comprise less than about 9 g, less than about 10 g, less than about 15 g, less than about 20 g, less than about 25 g, less than about 30 g, less than about 35 g, less than about 40 g, less than about 45 g, less than about 50 g, less than about 55 g, or less than about 60 g of carbohydrates per day. In some embodiments, an insulin suppressing diet can comprise less than about 10 g of carbohydrates per day. In some embodiments, an insulin suppressing diet can comprise less than about 20 g of carbohydrates per day. In some embodiments, an insulin suppressing diet can comprise less than about 30 g of carbohydrates per day. In some embodiments, an insulin suppressing diet can comprise less than about 40 g of carbohydrates per day.

In some embodiments, an insulin suppressing diet can comprise from about 0.1-10% or from about 1-10% of total calories coming from carbohydrates. In some embodiments, an insulin suppressing diet can comprise less than about 1%, less than about 2%, less than about 3%, less than about 4%, less than about 5%, less than about 6%, less than about 7%, less than about 8%, less than about 9%, or less than about 10% of total calories coming from carbohydrates. In some embodiments, an insulin suppressing diet can comprise less than about 2% of total calories coming from carbohydrates. In some embodiments, an insulin suppressing diet can comprise less than about 3% of total calories coming from carbohydrates. In some embodiments, an insulin suppressing diet can comprise less than about 4% of total calories coming from carbohydrates.

An insulin suppressing diet of the disclosure can promote foods with moderate levels of protein. In some embodiments, an insulin suppressing diet can comprise from about 0.8 g to about 1 g, from about 0.25 g to about 0.5 g, from about 0.5 g to about 0.75 g, from about 0.75 g to about 1 g, from about 1 g to about 1.25 g, or from about 1.25 g to about 1.5 g of protein per kilogram of reference weight of a subject per day. In some embodiments, an insulin suppressing diet can comprise from about 0.75 g to about 1.0 g of protein per kilogram of reference weight of a subject per day. In some embodiments, an insulin suppressing diet can comprise from about 0.8 g to about 1.0 g of protein per kilogram of reference weight of a subject per day. In some embodiments, an insulin suppressing diet can comprise from about 1 g to about 1.25 g of protein per kilogram of reference weight of a subject per day.

In some embodiments, an insulin suppressing diet can comprise about 0.4 g, about 0.5 g, about 0.6 g, about 0.7 g, about 0.8 g, about 0.9 g, about 1.0 g, about 1.1 g, about 1.2 g, about 1.3 g, about 1.4 g, or about 1.5 g of protein per kilogram of reference weight of a subject per day. In some embodiments, an insulin suppressing diet can comprise about 0.7 g of protein per kilogram of reference weight of a subject per day. In some embodiments, an insulin suppressing diet can comprise about 0.8 g of protein per kilogram of reference weight of a subject per day. In some embodiments, an insulin suppressing diet can comprise about 0.9 g of protein per kilogram of reference weight of a subject per day. In some embodiments, an insulin suppressing diet can comprise about 1.0 g of protein per kilogram of reference weight of a subject per day. In some embodiments, an insulin suppressing diet can comprise about 1.1 g of protein per kilogram of reference weight of a subject per day. In some embodiments, an insulin suppressing diet can comprise about 1.2 g of protein per kilogram of reference weight of a subject per day.

In some embodiments, an insulin suppressing diet can comprise from about 1-20%, from about 5-15%, or from 1-10% of total calories coming from protein. In some embodiments, an insulin suppressing diet can comprise about 5%, about 6%, about 7%, about 8%, about 9%, about 10%, about 11%, about 12%, about 13%, about 14%, about 15%, about 16%, about 17%, about 18%, about 19%, or about 20% of total calories coming from protein. In some embodiments, an insulin suppressing diet can comprise about 8% of total calories coming from protein. In some embodiments, an insulin suppressing diet can comprise about 9% of total calories coming from protein. In some embodiments, an insulin suppressing diet can comprise about 10% of total calories coming from protein. In some embodiments, an insulin suppressing diet can comprise about 11% of total calories coming from protein. In some embodiments, an insulin suppressing diet can comprise about 12% of total calories coming from protein.

An insulin suppressing diet can be high in fat. In some embodiments, an insulin suppressing diet can comprise from about 70% to about 75%, from about 75% to about 80%, from about 80% to about 85%, from about 85% to about 90%, or from about 90% to about 95% of total calories coming from fat. In some embodiments, an insulin suppressing diet can comprise from about 70% to about 80% of total calories coming from fat. In some embodiments, an insulin suppressing diet can comprise from about 80% to about 90% of total calories coming from fat.

An insulin suppressing diet can be high in fat. In some embodiments, an insulin suppressing diet can comprise about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, or about 94% of total calories coming from fat. In some embodiments, an insulin suppressing diet can comprise about 80% of total calories coming from fat. In some embodiments, an insulin suppressing diet can comprise about 85% of total calories coming from fat. In some embodiments, an insulin suppressing diet can comprise about 87% of total calories coming from fat. In some embodiments, an insulin suppressing diet can comprise about 90% of total calories coming from fat.

In some embodiments, an insulin suppressing diet of the disclosure can have a mass ratio of [fat grams]: [grams of protein+net carbohydrates]. In some embodiments, the mass ratio of [fat grams]: [grams of protein+net carbohydrates] is about 5:1, about 4:1, about 3:1, about 2:1, about 1:1, about 1:2, about 1:3, about 1:4, or about 1:5. In some embodiments, the mass ratio of [fat grams]: [grams of protein+net carbohydrates] is about 4:1. In some embodiments, the mass ratio of [fat grams]: [grams of protein+net carbohydrates] is about 3:1. In some embodiments, the mass ratio of [fat grams]: [grams of protein+net carbohydrates] is about 2:1.

An insulin suppressing diet can limit the total number of daily calories. The number of kilocalories per kilogram of reference weight in an insulin suppressing diet can depend on the body mass index (BMI) of a subject. In some embodiments, a subject has a BMI<30 kg/m²), and the insulin suppressing diet comprises from about 20 to about 25 kcal/kg, from about 25 to about 30 kcal/kg, from about 30 to about 35 kcal/kg, from about 35 to about 40 kcal/kg, from about 40 to about 45 kcal/kg, or from about 45 to about 50 kcal/kg. In some embodiments, a subject has a BMI<30 kg/m²), and the insulin suppressing diet comprises from about 25 to about 30 kcal/kg. In some embodiments, a subject has a BMI<30 kg/m²), and the insulin suppressing diet comprises from about 30 to about 35 kcal/kg. In some embodiments, a subject has a BMI<30 kg/m²), and the insulin suppressing diet comprises from about 35 to about 40 kcal/kg. In some embodiments, a subject has a BMI>30 kg/m²), and the insulin suppressing diet comprises from about 20 to about 25 kcal/kg, from about 25 to about 30 kcal/kg, from about 30 to about 35 kcal/kg, from about 35 to about 40 kcal/kg, from about 40 to about 45 kcal/kg, or from about 45 to about 50 kcal/kg. In some embodiments, a subject has a BMI>30 kg/m²), and the insulin suppressing diet comprises from about 20 to about 25 kcal/kg. In some embodiments, a subject has a BMI>30 kg/m²), and the insulin suppressing diet comprises from about 25 to about 30 kcal/kg. In some embodiments, a subject has a BMI>30 kg/m²), and the insulin suppressing diet comprises from about 30 to about 35 kcal/kg.

In some embodiments, methods of the disclosure further comprise administering a multivitamin. In some embodiments, the multivitamin provides the subject with the daily recommended amounts of nutrients, for example, vitamin C, vitamin D, vitamin E, riboflavin, vitamin B-6, folate or L-methyl folate, vitamin B-12, biotin, calcium, zinc, or omega-3 fatty acids.

Lower insulin and nutritional ketosis can trigger increased renal excretion of sodium and fluid. Thus, ensuring adequate sodium intake can be required in conjunction with administering an insulin suppressing diet described herein. In some embodiments, methods of the disclosure further comprise ensuring adequate sodium intake by the subject. In some embodiments, methods of the disclosure further comprise limiting the amount of sodium consumed by the subject. In some embodiments, sodium intake of a subject is from about 2 g to about 2.5 g, from about 2.5 g to about 3 g, from about 3 g to about 3.5 g, from about 3.5 g to about 4 g, from about 4 g to about 4.5 g, from about 4.5 g to about 5 g, from about 5 g to about 5.5 g, or from about 5.5 g to about 6 g per day. In some embodiments, sodium intake of a subject is maintained to an amount of from about 3.5 g to about 4 g per day. In some embodiments, sodium intake of a subject is maintained to an amount of from about 4 g to about 4.5 g per day. In some embodiments, sodium intake of a subject is maintained to an amount of from about 4 g to about 5 g per day. In some embodiments, sodium intake of a subject is maintained to an amount of from about 4.5 g to about 5 g per day. In some embodiments, an insulin suppressing diet described herein comprises a sodium intake amount of from about 2 g to about 2.5 g, from about 2.5 g to about 3 g, from about 3 g to about 3.5 g, from about 3.5 g to about 4 g, from about 4 g to about 4.5 g, from about 4.5 g to about 5 g, from about 5 g to about 5.5 g, or from about 5.5 g to about 6 g per day. In some embodiments, an insulin suppressing diet described herein comprises a sodium intake amount of from about 3.5 g to about 4 g per day. In some embodiments, an insulin suppressing diet described herein comprises a sodium intake amount of from about 4 g to about 4.5 g per day. In some embodiments, an insulin suppressing diet described herein comprises a sodium intake amount of from about 4 g to about 5 g per day. In some embodiments, an insulin suppressing diet described herein comprises a sodium intake amount of from about 4.5 g to about 5 g per day.

### Methods of Use

The present disclosure relates generally to compositions and methods for treating a disease or disorder associated with PI3K signaling. In one embodiment, a disease or disorder associated with PI3K signaling is a cell proliferative disease. In one embodiment, a disease or disorder associated with PI3K signaling is a neurodegenerative disease. In one embodiment, a disease or disorder associated with PI3K signaling is an inflammatory disease or condition. In one embodiment, a disease or disorder associated with PI3K signaling is a metabolic disease.

In some embodiments, the disease or disorder associated with PI3K signaling is a cell proliferative disease. In some embodiments, compounds of the disclosure can be used to treat a cancer in a subject. A compound of the disclosure can, for example, slow the proliferation of cancer cell lines, or kill cancer cells. Non-limiting examples of cancer that can be treated by a method of the disclosure includes one or more leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphomas (Hodgkin's disease, non-Hodgkin's disease), Waldenstrom's macroglobulinemia, heavy chain disease, and solid tumors such as sarcomas and carcinomas (e.g., fibrosarcoma, myosarcoma, liposarcoma, chondrosarcoma, osteogenic sarcoma, chordoma, angiosarcoma, endotheliosarcoma, lymphangiosarcoma, lymphangio endotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma), or a combination thereof. In some embodiments, the compounds of the disclosure show non-lethal toxicity.

In some embodiments, the cell proliferative disease is a solid tumor. In some embodiments, the cell proliferative disease is an advanced solid tumor.

In some embodiments, the cell proliferative disease is colorectal cancer. Colorectal cancer is cancer that starts in either the colon or the rectum and is also known as either colon cancer or rectal cancer.

Newly diagnosed metastatic CRC can be treated with 5-fluorouracil (5-FU)-based chemotherapy regimens, such as FOLFOX (5-FU, oxaliplatin, and leucovorin) or FOLFIRI (5-FU, irinotecan, and leucovorin). Chemotherapy can be used alone or in combination with targeted therapies such as those that block epidermal growth factor receptor (EGFR) or vascular endothelial growth factor (VEGF) signaling. For example, the addition of bevacizumab (anti-VEGF antibody) to FOLFIRI can increase overall survival (OS). In subsequent lines of therapy, responses can be poor. In this setting, targeted therapies like regorafenib and trifluridine/tipiracil can be used to maximize OS. In some embodiments, a method for treating CRC in a subject comprises administration of an inhibitor of the insulin-receptor/PI3K/AKT/mTOR pathway in combination with a SOC therapy for CRC such as those described herein.

In some embodiments, the cell proliferative disease is ovarian cancer (OC). In some embodiments, the cell proliferative disease is endometrial cancer (EC). In some embodiments, the cell proliferative disease is endometrial adenocarcinoma. In some embodiments, the cell proliferative disease is ovarian endometroid carcinoma. In some embodiments, the cell proliferative disease is ovarian clear cell carcinoma.

For advanced or recurrent EC, combination chemotherapy with carboplatin and paclitaxel is the SOC. Molecular characterization of EC can be critical in directing treatment for advanced and recurrent disease. Assessment of estrogen receptor and progesterone receptor status, microsatellite instability analysis, and assessment of human epidermal growth factor receptor 2 (HER2) status for uterine serous cancers can be key in addition to histologic and genetic analysis. For example, subject with advanced or recurrent endometrioid endometrial tumors and are positive for estrogen receptors and progesterone receptors can be treated with hormonal agents like progesterone if chemotherapy is intolerable. Combinations of antihormonal and biologic agents can also be effective as second- or third-line treatment for endometrioid ECs. For example, the combination of everolimus and letrozole can be used. Subjects with advanced stage uterine serous carcinomas that overexpress HER2, trastuzumab added to carboplatin and paclitaxel can also be used to prolong progression free survival (PFS) and OS.

Advanced OC is commonly treated with chemotherapy, particularly platinum-based chemotherapy. OC can typically be highly responsive to chemotherapy; however, OC can often recur quickly. Cancer that recurs more than six months after the end of treatment is defined as “platinum-sensitive,” whereas cancer that recurs less than six months after the end of treatment is defined as “platinum-resistant.” Subjects with platinum-resistant disease can have a worse prognosis than those with platinum-sensitive disease.

Germline (g) alterations in breast cancer 1 (gBRCA1) and breast cancer 2 (gBRCA2) genes and somatic mutations have been identified in women diagnosed with EOC. These EOCs also exhibit homologous recombination deficiency (HRD) involved in repair of DNA damage and replication. Poly(ADP-ribose) polymerase inhibitors (PARPis) can be used for EOC management across the treatment life cycle. Newly diagnosed stage III-IV EOC whose disease is in complete or partial response to first-line, platinum-based chemotherapy. High-grade serous or endometrioid EOC can be treated with PARPi maintenance therapy with niraparib. Subjects with germline or somatic pathogenic or likely pathogenic variants in BRCA1 (g/sBRCA1) or BRCA2 (g/sBRCA2) genes can be treated with olaparib. The addition of olaparib to bevacizumab can be used to subjects with stage III-IV EOC with g/sBRCA1/2 and/or genomic instability and a partial or complete response to chemotherapy plus bevacizumab combination. Maintenance therapy (second line or more) with single-agent PARPi can be used for subjects with EOC who have not received a PARPi and have responded to platinum-based therapy regardless of BRCA mutation status. Treatment with a PARPi can be used to subjects with recurrent EOC that has not recurred within 6 months of platinum-based therapy, who have not received a PARPi and have a g/sBRCA1/2, or whose tumor demonstrates genomic instability. Antiangiogenics such as bevacizumab can be used in conjunction with chemotherapy and PARPis to prevent the growth of blood vessels into the tumor. While these advances have significantly impacted the upfront treatment of subjects with OC, response rates to available cytotoxic therapies for recurrent OC remain poor, and an unmet need to exploit the molecular aberrations in rare subtypes such as ovarian endometrioid and ovarian clear cell carcinomas clearly exists.

In some embodiments, a method for treating an endometrial or ovarian cancer in a subject comprises administration of an inhibitor of the insulin-receptor/PI3K/AKT/mTOR pathway in combination with a SOC therapy for the endometrial or ovarian cancer, such as those described herein.

In some embodiments, the disease or disorder associated with PI3K signaling is a neurodegenerative disease including, but not limited to, brain trauma, spinal cord trauma, trauma to the peripheral nervous system, Alzheimer's disease, Pick's disease, diffuse Lewy body disease, progressive supranuclear palsy (Steel-Richardson syndrome), multisystem degeneration (Shy-Drager syndrome), motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffman disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia, age-related dementia, vascular dementia, diffuse white matter disease (Binswanger's disease), dementia of endocrine or metabolic origin, dementia of head trauma and diffuse brain damage, dementia pugilistica or frontal lobe dementia, neurodegenerative disorders resulting from cerebral ischemia or infraction including embolic occlusion and thrombotic occlusion as well as intracranial hemorrhage of any type, intracranial and intravertebral lesions, hereditary cerebral angiopathy, hereditary amyloid, Down's syndrome, macroglobulinemia, secondary familial Mediterranean fever, Muckle-Wells syndrome, multiple myeloma, pancreatic-related amyloidosis, cardiac-related amyloidosis, chronic hemodialysis arthropathy, Finnish amyloidosis, Iowa amyloidosis, or a combination thereof.

In some embodiments, the disease or disorder associated with PI3K signaling is an inflammatory disorder including, but not limited to, Type II diabetes, insulin resistance cardiovascular disease, arrhythmia, atherosclerosis, coronary artery disease, hypertriglyceridemia, dyslipidemia, retinopathy, nephropathy, neuropathy, obesity, and macular edema, ileitis, ulcerative colitis, Barrett's syndrome, Crohn's disease, or a combination thereof.

In some embodiments, the disease or disorder associated with PI3K signaling is a metabolic disease including, but not limited, Type II diabetes, insulin resistance cardiovascular disease, arrhythmia, atherosclerosis, coronary artery disease, hypertriglyceridemia, dyslipidemia, retinopathy, nephropathy, neuropathy, obesity, macular edema, or a combination thereof.

In some embodiments, the methods of the disclosure further comprise administration of a modulator of glucose metabolism, use of a diet that influences the subject's metabolic state, or a combination thereof.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

### Therapeutic Effects

Disclosed herein is a method of treating a cancer in a subject in need thereof. In some embodiments, the methods of the disclosure can decrease cell proliferation and/or decrease tumor size.

The methods of the disclosure can decrease cell proliferation in a subject. In some embodiments, the methods of the disclosure can decrease cell proliferation in a subject by from about 5% to about 10%, from about 10% to about 15%, from about 15% to about 20%, from about 20% to about 25%, from about 25% to about 30%, from about 35% to about 40%, from about 40% to about 45%, from about 45% to about 50%, from about 50% to about 75%, from about 75% to about 100%, from about 100% to about 125%, from about 125% to about 150%, from about 150% to about 175%, or from about 175% to about 200% compared to a subject that is not administered a therapeutic agent or insulin suppressing diet. In some embodiments, the methods of the disclosure can decrease cell proliferation in a subject by from about 20% to about 25% compared to a subject that is not administered the therapeutic agent or the insulin suppressing diet. In some embodiments, methods of the disclosure can decrease cell proliferation in a subject by from about 50% to about 75% compared to a subject that is not administered the therapeutic agent or the insulin suppressing diet.

In some embodiments, methods of the disclosure can decrease cell proliferation in a subject by about 5%, about 10%, about 15%, about 20%, about 25%, about 30%, about 35%, about 40%, about 45%, about 50%, about 75%, about 100%, about 125%, about 150%, about 175%, or about 200% compared to a subject that is not administered the therapeutic agent or the insulin suppressing diet. In some embodiments, the methods of the disclosure can decrease cell proliferation in a subject by about 20% compared to a subject that is not administered the therapeutic agent or the insulin suppressing diet. In some embodiments, the methods of the disclosure can decrease cell proliferation in a subject by about 30% compared to a subject that is not administered the therapeutic agent or the insulin suppressing diet. In some embodiments, the methods of the disclosure can decrease cell proliferation in a subject by about 50% compared to a subject that is not administered the therapeutic agent or the insulin suppressing diet. In some embodiments, the methods of the disclosure can decrease cell proliferation in a subject by about 70% compared to a subject that is not administered the therapeutic agent or the insulin suppressing diet.

In some embodiments, methods of the disclosure can decrease a tumor size in a subject by from about 5% to about 10%, from about 10% to about 15%, from about 15% to about 20%, from about 20% to about 25%, from about 25% to about 30%, from about 35% to about 40%, from about 40% to about 45%, from about 45% to about 50%, from about 50% to about 75%, from about 75% to about 100%, from about 100% to about 125%, from about 125% to about 150%, from about 150% to about 175%, or from about 175% to about 200%. In some embodiments, methods of the disclosure can decrease a tumor size in a subject by from about 20% to about 25%. In some embodiments, methods of the disclosure can decrease a tumor size in a subject by from about 45% to about 50%.

In some embodiments, methods of the disclosure can decrease a tumor size in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, methods of the disclosure can decrease a tumor size in a subject by about 20%. In some embodiments, methods of the disclosure can decrease a tumor size in a subject by about 30%. In some embodiments, methods of the disclosure can decrease a tumor size in a subject by about 50%.

The methods of the disclosure can increase overall survival of a subject. In some embodiments, methods of the disclosure can increase overall survival of a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, methods of the disclosure can increase overall survival of a subject by at least about 10%. In some embodiments, methods of the disclosure can increase overall survival of a subject by at least about 20%. In some embodiments, methods of the disclosure can increase overall survival of a subject by at least about 30%.

Methods of the disclosure can increase progression free survival of a subject. In some embodiments, methods of the disclosure can increase progression free survival of a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, methods of the disclosure can increase progression free survival of a subject by at least about 10%. In some embodiments, methods of the disclosure can increase progression free survival of a subject by at least about 20%. In some embodiments, methods of the disclosure can increase progression free survival of a subject by at least about 30%.

Methods of the disclosure can increase percentage of cancer cell death. In some embodiments, methods of the disclosure can increase percentage of cancer cell death by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, methods of the disclosure can increase percentage of cancer cell death by at least about 10%. In some embodiments, methods of the disclosure can increase percentage of cancer cell death by at least about 20%. In some embodiments, methods of the disclosure can increase percentage of cancer cell death by at least about 30%.

The methods of the disclosure can increase sensitivity to a cancer therapy in a subject. In some embodiments, methods of the disclosure can increase sensitivity to a cancer therapy in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, methods of the disclosure can increase sensitivity to a cancer therapy in a subject by at least about 10%. In some embodiments, methods of the disclosure can increase sensitivity to a cancer therapy in a subject by at least about 20%. In some embodiments, methods of the disclosure can increase sensitivity to a cancer therapy in a subject by at least about 30%.

The methods of the disclosure can increase a treatment response rate of a therapeutic agent. In some embodiments, methods of the disclosure can increase a treatment response rate of a therapeutic agent by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, methods of the disclosure can increase a treatment response rate of a therapeutic agent by at least about 10%. In some embodiments, methods of the disclosure can increase a treatment response rate of a therapeutic agent by at least about 20%. In some embodiments, methods of the disclosure can increase a treatment response rate of a therapeutic agent by at least about 30%.

The methods of the disclosure can increase the efficacy of the therapeutic agent in a subject compared to a subject treated only with the insulin suppressing diet of the disclosure. In some embodiments, methods of the disclosure can increase the efficacy of the therapeutic agent in a subject by at least about at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% compared to a subject treated only with the insulin suppressing diet of the disclosure. In some embodiments, methods of the disclosure can increase the efficacy of the therapeutic agent in a subject by at least about at least about 10% compared to a subject treated only with the insulin suppressing diet of the disclosure. In some embodiments, methods of the disclosure can increase the efficacy of the therapeutic agent in a subject by at least about at least about 20% compared to a subject treated only with the insulin suppressing diet of the disclosure. In some embodiments, methods of the disclosure can increase the efficacy of the therapeutic agent in a subject by at least about at least about 30% compared to a subject treated only with the insulin suppressing diet of the disclosure.

The methods disclosed herein can have a decreased dose of the therapeutic agent compared to a subject treated with the therapeutic agent alone to achieve the same outcome. In some embodiments, methods of the disclosure can decrease the required dose of the therapeutic agent by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% compared to a subject treated with the therapeutic agent alone to achieve the same outcome. In some embodiments, methods of the disclosure can decrease the required dose of the therapeutic agent by at least about 10% compared to a subject treated with the therapeutic agent alone to achieve the same outcome. In some embodiments, methods of the disclosure can decrease the required dose of the therapeutic agent by at least about 20% compared to a subject treated with the therapeutic agent alone to achieve the same outcome. In some embodiments, methods of the disclosure can decrease the required dose of the therapeutic agent by at least about 30% compared to a subject treated with the therapeutic agent alone to achieve the same outcome.

The methods disclosed herein can decrease adverse events associated with the therapeutic agent compared to a subject treated with the therapeutic agent alone. In some embodiments, methods of the disclosure can decrease adverse events associated with the therapeutic agent by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% compared to a subject treated with the therapeutic agent alone. In some embodiments, methods of the disclosure can decrease adverse events associated with the therapeutic agent by at least about 10% compared to a subject treated with the therapeutic agent alone. In some embodiments, methods of the disclosure can decrease adverse events associated with the therapeutic agent by at least about 20% compared to a subject treated with the therapeutic agent alone. In some embodiments, methods of the disclosure can decrease adverse events associated with the therapeutic agent by at least about 30% compared to a subject treated with the therapeutic agent alone.

Methods and compositions disclosed herein can be used to increase the effectiveness of treatment with a pathway inhibitor (e.g., an inhibitor of the insulin-receptor/PI3K/AKT/mTOR signaling pathway). Thus, the disclosure provides a method for increasing the effectiveness or efficacy of treatment with a pathway inhibitor (e.g., an inhibitor of the insulin-receptor/PI3K/AKT/mTOR signaling pathway). Such a method can include treating a subject with an effective amount of the pathway inhibitor and optionally an effective amount of a modulator of glucose metabolism. The disclosure also provides a method for increasing the effectiveness or efficacy of treatment with a pathway inhibitor (e.g., an inhibitor of the insulin-receptor/PI3K/AKT/mTOR signaling pathway). Such a method can include treating a subject with an effective amount of the pathway inhibitor, wherein the subject consumes an insulin suppressing diet during treatment. The subject can be in need of such treatment. Alternatively, treatment can be used to reduce the incidence or onset of disease in the subject.

Methods and compositions disclosed herein may allow the use of a lower dosage of a pathway inhibitor. Thus, the disclosure provides a method for treating a disease or disorder associated with PI3K signaling, comprising administering to a subject in need thereof an effective amount of the pathway inhibitor (e.g., an inhibitor of the insulin-receptor/PI3K/AKT/mTOR signaling pathway) and optionally an effective amount of a modulator of glucose metabolism, wherein the effective amount of the pathway inhibitor is a lower amount than the amount effective in the absence of treatment with the modulator of glucose metabolism. The disclosure also provides a method for treating a disease or disorder associated with PI3K signaling, comprising administering to a subject in need thereof an effective amount of the pathway inhibitor (e.g., an inhibitor of the insulin-receptor/PI3K/AKT/mTOR signaling pathway), wherein the subject consumes an insulin suppressing diet during treatment, wherein the effective amount of the pathway inhibitor is a lower amount than the amount effective in the absence of the subject consuming the insulin suppressing diet during treatment. In certain embodiments, the effective amount of the pathway inhibitor is less than 90%, less than 80%, less than 70%, less than 60%, less than 50%, less than 40%, or less than 30%, when used in combination with the modulator of glucose metabolism and/or insulin suppressing diet, than is the amount of the pathway inhibitor when used alone.

Methods and compositions disclosed herein may allow for less frequent administration of a pathway inhibitor. Thus, the disclosure provides a method for treating a disease or disorder associated with PI3K signaling, comprising administering to a subject in need thereof an effective amount of the pathway inhibitor (e.g., an inhibitor of the insulin-receptor/PI3K/AKT/mTOR signaling pathway) and an effective amount of a modulator of glucose metabolism, wherein the pathway inhibitor is administered less frequently than the frequency effective in the absence of treatment with the modulator of glucose metabolism. The disclosure also provides a method for treating a disease or disorder associated with PI3K signaling, comprising administering to a subject in need thereof an effective amount of a pathway inhibitor (e.g., an inhibitor of the insulin-receptor PI3K/AKT/mTOR signaling pathway) wherein the subject consumes an insulin suppressing diet during treatment, wherein the pathway inhibitor is administered less frequently than the frequency effective in the absence of the subject consuming the insulin suppressing diet during treatment. For example, at least one, or at least two, or at least three, or at least four, or at least five, or at least six, or at least seven, or at least eight, or at least ten, or at least thirteen, or at least fifteen fewer doses of the pathway inhibitor and/or the modulator of glucose metabolism can be administered over a duration of treatment than when the pathway inhibitor or the modulator of glucose metabolism is administered alone.

### EXAMPLES

### Example 1: Formulations of the Insulin Suppressing Diet (Study ISD)

A wide range of whole foods are incorporated into the Study ISD menu including non-starchy vegetables, meats (beef, chicken, pork, fish, lamb), nuts and seeds, oils (olive, avocado, canola, coconut), cheeses, butter, cream, whole eggs, and small amounts of fruits (berries, olives, avocado, tomatoes, lemons/limes). Instructions are given on food storage and reheating attached to the food log for each day. Meal formulation is adjusted in a manner that allows for ideal protein allotments for each person to be met with additional ‘optional’ calories provided as fat. Similarly, appropriate micronutrient quantities in the meal formulation are ensured. No preservatives or any other artificial chemicals are used in any meal.

The Study ISD comprises, or in some cases consists of, the following target intakes: 30 to 40 g of carbohydrates per day; between 0.8 and 1.0 g of protein per kg of reference weight of a subject; and 30-35 kcal/kg (BMI <30 kg/m² or 25-30 kcal/kg (BMI >30 kg/m²). Alternatively, the Study ISD comprises, or in some cases consists of, the following target intakes: less than about 9 g of carbohydrates per day; between 0.8 and 1.0 g of protein per kg of reference weight of a subject; and 30-35 kcal/kg (BMI <30 kg/m² or 25-30 kcal/kg (BMI >30 kg/m²).

The Study ISD is a very low carbohydrate, high fat diet formulated from whole, fresh food products. The meals and snacks are designed to provide a 3:1 ratio of fat grams to grams protein and net carbohydrate, with 87% of total calories from fat, 10% from protein, and 3% from carbohydrate. Alternatively, the meals and snacks are designed to provide a 3:1 ratio of fat grams to grams protein and net carbohydrate, with 82% of total calories from fat, 11% from protein, and 7% from carbohydrate. Meals and snacks provide adequate calories for weight maintenance and are adjusted to provide approximately 30-35 kcal/kg body weight for subjects with BMI <30 kg/m² and 25-30 kcal/kg for subjects with BMI >30 kg/m². The diet provides approximately 0.8 to 1.0 g protein/kg body weight for both obese and more lean subjects.

Subjects are provided with a eucaloric number of calories, defined as food portions adjusted to meet adequate calories for weight maintenance (30-35 kcal/kg for a BMI <30 kg/m² and 25-30 kcal/kg for a BMI >30 kg/m²). For example, a 70 kg non-obese woman is fed 2,100 kcal per day composed of 35 g of carbohydrate, 56 g of protein, and 193 g of fat. Obese individuals consuming an ISD naturally restrict caloric intake resulting in fat loss, while preserving functional lean body mass. Subjects are asked to eat to satiety. Subjects record the percentage of food eaten at each meal/snack in a log that is returned to the study investigators. At regular follow-up intervals, subjects are weighed and given a food log to assess water intake, compliance, and tolerance to the dietary interventions.

The Study ISD contains adequate essential vitamins and minerals achieved through consumption of a wide variety of whole foods prepared using appropriate methods to preserve nutrients. To ensure that all nutritional goals are met, the meal plans are supplemented with a multivitamin to meet 100% of recommended needs. Ensuring adequate sodium intake is particularly important since lower insulin and nutritional ketosis trigger increased renal excretion of sodium and fluid. The exact amount of additional sodium needed on an ISD varies based on several factors. As a general guide, if an individual normally consumes about 3 g sodium/day, an extra 1-2 g for a total of 4-5 g/day is a good starting place. Adequate sodium and potassium intake is achieved through selection of appropriate food sources and cooking methods and monitoring of symptoms for micronutrient deficiencies (e.g., sodium, potassium, magnesium) are used to avoid common side effects and optimize acceptability and adherence.

The Study ISD formulation is consistent with other very low carbohydrate diets that have been used safely in thousands of adults and children. Constipation, thirst/dehydration, frequent urination, fatigue, tachycardia, lightheadedness, weight loss, and increased cholesterol are common (>5%). People on this diet for months or years can also have decreases in some vitamins and minerals such as magnesium, calcium, vitamin D, and folate. Thus, standard once a day vitamin supplements are given. Subjects receive meals based on a two-week meal plan. All subjects receive written documents which include the list of ingredients and nutritional labels, per FDA guidelines, along with full heating and meal storage instructions. Instructions are given on food storage and reheating with each shipment.

### Example 2: Use of Serabelisib in Combination with an Insulin Suppressing Diet in Subjects with Cancer

The primary objective of the study is to evaluate the safety, feasibility, and pharmacokinetic impact of intrasubject modulated dosing for serabelisib when co-administered with a study ISD in a pilot cohort of subjects with solid tumors with PIK3CA mutations with or without PTEN loss. Serabelisib, a selective small-molecule inhibitor of PI3Kα, is investigated for clinical safety, tolerability, and efficacy of subjects with advanced solid tumors with phosphatidylinositol-4,5-bisphosphate 3-kinase catalytic alpha (PI3Kα or PIK3CA) mutations and a subset who also have phosphatase and tensin homolog (PTEN) loss.

Combining PI3K inhibition with an insulin suppressing diet (Study ISD) results in improved efficacy across many tumor types compared to PI3K inhibition alone. This study applies to human subjects with cancer. Specifically, the combination of serabelisib, a PI3K alpha isoform (PI3Kα) inhibitor, and the Study ISD is evaluated to determine safety and efficacy in adult subjects with advanced, PIK3CA-mutated solid tumors with or without PTEN loss.

General Methods: There are fifty total subjects in this study, split into four cohorts. The first cohort comprises eight subjects with PIK3CA mutations, with or without PTEN loss, in solid tumors. The cohort is used to determine the optimal Serabelisib dose when combined with an ISD. The second cohort comprises fourteen subjects with PIK3CA mutations, with or without PTEN loss, in colorectal cancer. The third cohort comprises fourteen subjects with PIK3CA mutations, with or without PTEN loss, in endometrial cancer. The fourth cohort comprises fourteen subjects with PIK3CA mutations, with or without PTEN loss, in ovarian endometroid or clear cell carcinoma. The expansion cohorts two, three, and four evaluate the efficacy of combining Serabelisib with the insulin suppressing diet.

The study takes place in up to 20 study sites in the United States. The total planned enrollment is approximately 50 subjects, who are ≥18 years old and have histologically or cytologically confirmed recurrent solid tumors with PIK3CA mutations with or without PTEN loss. Each subject enrolled participates in the study under a single cohort. The general objectives of the studies are to assess the antitumor efficacy, the pharmacokinetics (PK), and the pharmacodynamics (PD) of serabelisib in combination with a study ISD in subjects with solid tumors with PIK3CA mutations with or without PTEN loss.

Preparation, Handling, and Storage of Serabelisib: Serabelisib drug product is an anticancer drug. As with other potentially toxic compounds, caution is exercised when handling the serabelisib tablets. Serabelisib is administered as provided and is not manipulated (e.g., cut, crushed, chewed, or mixed with food) in any way. Serabelisib drug product is stored at room temperature (15° C. to 30° C.; 59° F. to 86° F.). All study supplies are kept in a restricted access area.

At a minimum, each Study Drug label of serabelisib shipped to the sites provides the following information: batch number/lot number, study identification, required storage conditions, directions for use, and caution statements. The serabelisib label includes “New Drug-Limited by United States federal law to investigational use” language. Serabelisib tablets are packaged in high-density polyethylene bottles equipped with child-resistant cap and induction sealed.

Dosing and Administration: Subjects are provided with a sufficient quantity of the Study Drug, serabelisib, at the time points designated in the schedule of events. Serabelisib is taken PO QD orally in the morning. Study drug is taken on the same 3 consecutive days of each week (starting on either a Monday, Tuesday, or Wednesday) followed by four consecutive days off Study Drug. The Study Drug is administered in 28-day (4-week; +3 days) cycles without a break between cycles.

At Visit 3 (Cycle 1, Study Day 1), Visit 4 (Cycle 1, Study Day 9), Visit 5 (Cycle 1, Study Day 10), Visit 6.1 (Cycle 2, Study Day 37; Cohort 1 only), and Visit 6.2 (Cycle 2, Study Day 38; Cohort 1 only) Study Drug is taken by the subject in the clinic after fasting labs. On these clinic days, the subject is counseled not to take their Study Drug dose at home. Subjects are required to fast overnight (nothing to eat or drink except water, for at least 6 hours) prior to all study visits.

Subjects are provided the following dosing instructions:
1. Eat Study ISD breakfast as directed and try to start breakfast at approximately the same time each day.
2. Swallow the appropriate number of 300 mg serabelisib tablets (depending on cohort and dose assignment) with water within 15-20 minutes upon finishing breakfast. Tablets cannot be cut, chewed, or broken apart in any way; the whole tablet must be swallowed.
3. Record details of dosing.

If there is a missed Study Drug dose within 24 hours of the intended schedule, the subject takes the missed dose as soon as possible and no later than 24 hours after the scheduled dosing time. A late Study Drug dose is taken after lunch or dinner, but the study drug must be taken following Study ISD consumption.

If there is a missed Study Drug dose beyond 24 hours of the intended schedule, the study drug dose is not doubled on a single day due to the missed dose. There must not be fewer than 3 days off the Study Drug between the end of dosing for one week and the beginning of dosing the next week. The Study Drug schedule is moved up by one day to account for a missed dose (for example, with a M/T/W schedule, Study Drug missed on Tuesday can be taken on Wednesday and Thursday that week) except for cycle 1, week 2 (all cohorts) and cycle 2, week 2 (Cohort 1 only; due to PK timed procedures). The original dosing schedule is reinstated the following week.

Efficacy Assessments: Tumor Assessments can include tumor imaging which may consist of radiological assessments using CT (with contrast) or MRI scans of chest, abdomen, and/or pelvis. Objective response rate (CR or PR) and PFS may be assessed using RECIST 1.1. Physical examinations may include, but are not limited to, recording evaluations of the following body systems: general appearance, head (ear, nose, and throat), eyes, respiratory, cardiovascular, abdomen, urogenital, musculoskeletal, neurological, lymph nodes, and skin. ECOG PS can be recorded at screening and/or upon the first visit of each Study Drug Administration Cycle. Body weight can be recorded at screening and/or consistently throughout the study. Physical examination abnormalities identified at screening can be documented in the subject's source documents and on the medical history eCRF. Changes after the Screening Visit can be captured as AEs on the AE eCRF page.

Vital sign measurements can include, but are not limited to, blood pressure (e.g., diastolic, systolic), heart rate, respiratory rate, and temperature. Resting vital sign measurements can be performed after the subject has been at rest for a minimum of 5 minutes. Vital signs can be measured prior to any blood draws that occur at the same study visit. Blood pressure may be measured manually or with an automated device, either in the dominant or in the non-dominant arm. The same measurement technique or different measurement techniques for all vital sign evaluations can be used throughout the study for each subject.

Hematology clinical laboratory tests may include a CBC with differential (e.g., red blood cells [RBC], white blood cells (WBC), platelets, hemoglobin, neutrophils, lymphocytes, monocytes, basophils, eosinophils, mean corpuscular volume (MCV), mean corpuscular hemoglobin (MCH), mean corpuscular hemoglobin concentration (MCHC), and red cell distribution width (RDW). Serum chemistry clinical laboratory tests may test for BUN, creatinine, ALT, AST, ALP, LDH, total bilirubin, direct bilirubin, total protein, sodium, potassium, chloride, bicarbonate, calcium, phosphate, and magnesium. Urinalysis clinical laboratory tests may test for Dipstick protein, glucose, ketones, and blood; bilirubin, urobilinogen, pH, specific gravity, nitrite, and leukocyte esterase. A microscopic examination can also be performed, if necessary. A serum pregnancy test (for human chorionic gonadotropin [HCG]) may be performed on female subjects of childbearing potential at and/or after screening. Other laboratory tests can include, but are not limited to, HbA1c, blood ketones, glycated albumin, blood glucose, insulin, C-peptide, and continuous glucose monitoring.

Electrocardiogram parameters may include, but are not limited to, RR interval duration or heart rate, PR interval duration, QRS interval duration, QT interval duration, and QT interval corrected by Fredericia (QTcF). Single 12-lead ECGs can be recorded after the subject has been at rest for a minimum of 1 minute, 2 minutes, 3 minutes, 4 minutes, 5 minutes, 6 minutes, 7 minutes, 8 minutes, 9 minutes, 10 minutes, or more minutes. TABLE 2 depicts the clinical schedule for investigators. TABLE 3 depicts the clinical schedule for subjects.

TABLE 2
Schedule of Activities for Investigators

| | | Intervention Period | | | | | | | | | | |
|---|---|---|---|---|---|---|---|---|---|---|---|---|
| | | | Study Drug Administration Period | | | | | | | | | |
| | | | | | | | | | | | Cycle 4 | |
| | | | Cycle 1 | | | Cycle 2 | | | | Cycle 3 | onward | End of |
| | SC | RI | W 1 | W 2 | | W 1 | W 2¹ | | W 3 | W 1 | W 1 | Intervention |
| | Visit (study day) | | | | | | | | | | | (within |
| | V1 | V2 | | | | | | | | | | 28 d of |
| | (D −28 | (D −7 | V3 | V4 | V5 | V6 | V6.1¹ | V6.2¹ | V7 | V8 | V9 | last |
| Procedures | to −8) | to −1) | (D 1) | (D 9) | (D 10) | (D 29) | (D 37) | (D 38) | (D 43) | (D 57) | (D 85+) | dose) |
| Written | X | | | | | | | | | | | |
| informed | | | | | | | | | | | | |
| consent² | | | | | | | | | | | | |
| Inclusion and | X | | | | | | | | | | | |
| exclusion | | | | | | | | | | | | |
| criteria³ | | | | | | | | | | | | |
| Medical | X | | | | | | | | | | | |
| history | | | | | | | | | | | | |
| (including | | | | | | | | | | | | |
| demographics) | | | | | | | | | | | | |
| Physical | X | | X | | | X | | | | X | X | |
| examination | | | | | | | | | | | | |
| Height, | X | X⁴ | X^{4,5} | X⁴ | X⁴ | X^{4,5} | X⁴ | X⁴ | X⁴ | X^{4,5} | X^{4,5} | X^{4,5} |
| weight, ECOG | | | | | | | | | | | | |
| PS | | | | | | | | | | | | |
| Vital signs⁶ | X | X | X | X | X | X | X⁷ | X | X | X | X | X |
| 12-lead ECG | X | | X⁷ | X⁷ | | | X⁷ | | | | | |
| Continuous | | X⁸ | X⁹ | | X¹⁰ | X¹¹ | | | X¹² | X¹³ | | |
| glucose | | | | | | | | | | | | |
| monitoring | | | | | | | | | | | | |
| (FreeStyle | | | | | | | | | | | | |
| Libre) | | | | | | | | | | | | |
| placement/ | | | | | | | | | | | | |
| replacement/data | | | | | | | | | | | | |
| download | | | | | | | | | | | | |
| Dispense | | | X | | | X | | | X | X | X | |
| Serabelisib¹⁴ | | | | | | | | | | | | |
| Administer | | | X | X | X | | X | X | | | | |
| Serabelisib (in | | | | | | | | | | | | |
| clinic)¹⁵ | | | | | | | | | | | | |
| Food as | | X | X | X | X | X | X | X | X | X | X | |
| directed | | | | | | | | | | | | |
| (in clinic)¹⁶ | | | | | | | | | | | | |
| Adverse | | X (review on an ongoing basis) | | | | | | | | | | |
| events¹⁷ | | | | | | | | | | | | |
| Prior and | X | X (review on an ongoing basis) | | | | | | | | | | |
| concomitant | | | | | | | | | | | | |
| medications¹⁸ | | | | | | | | | | | | |
| Study drug | | | | | | X | | | X | X | X | X |
| reconciliation¹⁹ | | | | | | | | | | | | |

LABORATORY ASSESSMENTS²⁰

| | | | | | | | | | | | | |
|---|---|---|---|---|---|---|---|---|---|---|---|---|
| Pregnancy | X | | X | | | | | | | | | |
| test²¹ | | | | | | | | | | | | |
| Hematology | X | | | X | | X | X | | X | X | X | X |
| Chemistry | X | | X | X | | X | X | | X | X | X | X |
| Metabolic | X | X | X | X²² | X²³ | X | X²² | X²³ | X | X | X | X |
| HbA1c²⁴ | X | | | | | | | | | | X | X |
| Glycated | X | | X | | | X | | | X | X | X | X |
| albumin | | | | | | | | | | | | |
| Urinalysis | X | | X | X | | X | X | | X | X | X | X |

PHARMACOKINETIC ASSESSMENTS

| | | | | |
|---|---|---|---|---|
| PK sampling | X²⁵ | X²⁶ | X²⁵ | X²⁶ |

EFFICACY ASSESSMENTS

| | | | | | | |
|---|---|---|---|---|---|---|
| Circulating | X | X | X | X | X | X |
| Tumor | | | | | | |
| Markers²⁷ | | | | | | |
| Tumor | X²⁸ | | | X²⁸ | X²⁹ | |
| imaging | | | | | | |

PHARMACODYNAMIC ASSESSMENTS

| | | | | | |
|---|---|---|---|---|---|
| Tumor | X³⁰ | | | X³¹ | |
| biopsy/collection | | | | | |
| of archival | | | | | |
| tissue | | | | | |
| Buccal cell | | X | X³² | | X³² |
| collection | | | | | |

¹D 37 and D 38 (V6.1 and V6.2) for those in Cohort 1 only (for PK study upon dose escalation).
²Informed consent must be obtained prior to any study-specific Visit 1 (screening) activities.
³Perform check of inclusion and exclusion criteria at screening; confirm eligibility prior to Visit 2 (run-in).
⁴Body weight only.
⁵ECOG PS to be completed on the first visit of every cycle.
⁶Vital signs consist of heart rate, respiratory rate, temperature, and blood pressure.
⁷Day 1 (V3), Day 9 (V4), and Day 37 (V6.1; for Cohort 1 only) vital signs and 12-lead ECG to be performed pre-dose (up to 60 min prior to dosing) and then post-dose at 6 h (±30 min). 12-lead ECG to be performed in triplicate after 3 min rest in sitting position.
⁸Insert CGM sensor at clinic. Subjects are provided with two unopened CGM sensors (one for placement during visit and the other to remain unopened and taken home for backup) and education on how to remove sensor, return sensor to clinic, and replace sensor. Should the backup sensor be used at any point, another backup sensor must be provided.
⁹The sensor placed at visit 2 is removed, the data are downloaded, and a new one placed at least 1 h prior to taking Study Drug.
¹⁰Subjects are provided with two unopened CGM sensors; one is placed in clinic at least 1 h prior to the first planned administration of Study Drug and the other sent home with the subject along with education on how to remove sensor, store sensor for later return to clinic, and replace sensor with new one on day 24. Subjects are reminded via phone call on day 23.
¹¹Subjects return first sensor and remove second sensor provided at Visit 5. A new sensor is placed in clinic at least 1 h prior to taking Study Drug.
¹²Subjects remove sensor placed at Visit 6. A new sensor is placed in clinic at least 1h prior to taking Study Drug.
¹³Remove CGM sensor placed at Visit 7 at least 1h prior to taking Study Drug.
¹⁴Study drug (serabelisib) during Cycle 1 at Week 1 and Week 2 (and onward) to be administered PO QD in the morning on three consecutive days per week (either MTW, TWTh, or WThF of each week; subject is instructed to take Study Drug on the same 3 consecutive days of the week for the entire Study Drug administration). Study drug is be administered in 28-day (4-week; 28 ± 3 days) cycles without a break between cycles. Study drug is be dispensed in 300 mg tablets and must not be cut, broken, chewed, crushed, mixed with food/liquid before ingestion, or altered in any way. Where Study Drug is not indicated as taken in-clinic, it is taken at home. For all cohorts, serabelisib is taken within 20 min of finishing ISD breakfast.
¹⁵Serabelisib to be administered in clinic (due to timed PK sampling) within 20 min of finishing ISD breakfast. Subjects bring their dispensed serabelisib dose with them to all other clinic visits.
¹⁶Subjects bring study ISD breakfast (and lunch for V3 and V4) with them for consumption in clinic.
¹⁷AEs are collected at Visit 2 (non-intervention-related AEs) and then after the first administration of Study Drug until end of intervention (intervention-related AEs).
¹⁸Prior and concomitant medications are collected from 30 days prior to the first planned administration of Study Drug (prior medications) until end of intervention.
¹⁹Compliance with the dosing schedule is assessed by reconciliation of the used and unused Study Drug during the clinic visit on day 1 of each cycle, beginning with Cycle 2.
²⁰Laboratory assessments to be collected in a fasted state (assessments not required at V1 if collected in the past 14 d) and prior to Serabelisib dosing include:
a. Hematology: CBC with percent and absolute counts differential, Hb, MCV, MCHC, MCH, RDW, and platelet count.
b. Chemistry: BUN, creatinine, ALT, AST, ALP, total bilirubin, direct bilirubin, LDH, total protein, sodium, potassium, chloride, bicarbonate, calcium, phosphate, and magnesium
c. Metabolic: blood glucose, insulin, triglycerides, total cholesterol, LDL-direct, HDL, C-peptide, beta-hydroxybutyrate (BHB), and non-esterified fatty acids (NEFA)
d. Urinalysis: dipstick protein, glucose, ketones, and blood; bilirubin, urobilinogen, pH, specific gravity, nitrite, and leukocyte esterase. A microscopic examination is performed, if necessary.
²¹Pregnancy tests for female subjects of childbearing potential are performed at Visit 1 during screening (serum only) and at Visit 3 (urine). Day 1 (V3) urine pregnancy testing to be performed prior to dosing.
²²Serial assessment of glucose, serum insulin, C-peptide, BHB, NEFA pre-dose and post-dose at 1.5 h (±15 min), 3 h (±30 min), 6 h (±30 min), 9 h (±1 h), and 12 h (±1 h).
²³Assessment of glucose, serum insulin, C-peptide, BHB, and NEFA pre-dose and pre-study ISD breakfast.
²⁴HbA1c to be collected at V1 if not measured within 14 d prior to screening.
²⁵Serial assessment serabelisib PK pre-dose and post-dose at 1.5 h (±15 min), 3 h (±30 min), 6 h (±30 min), 9 h (±1 h), and 12 h (±1 h). Visit 4 occurs on the second day of serabelisib dosing and occurs no later than Thursday (to ensure the lab can receive PK samples).
²⁶Assessment of serabelisib PK pre-dose and pre-study ISD breakfast.
²⁷Circulating tumor marker(s) selected by the treating physician (such as CEA, CA 19-9, and CA 125) to be collected at screening and on the first visit of every Study Drug cycle for the first six months, and once every three Study Drug cycles thereafter.
²⁸CT scans with contrast of chest, abdomen, pelvis.
²⁹CT scans with contrast of chest, abdomen, pelvis to be conducted after completion of each even numbered cycle.
³⁰The screening procedures include evidence by a fresh tumor biopsy or archival tissue confirming mutational status for PIK3CA and PTEN. If mutational analysis of tumor tissue was not previously performed within the past 6 months, and if analysis did not include PIK3CA and PTEN loss, collection of available archival tumor tissue or fresh tumor tissue is to be sent to the local laboratory for determination of PIK3CA and PTEN mutational status, and PTEN immunohistochemistry. Tumor tissue biopsy is to be sent to the local laboratory for PD assessment of biomarkers (if only slides are available, then a minimum of 15 slides is required and they must have been collected within 6 months of screening); for Cohort 2, 3, and 4 subjects, the results of this procedure confirming PIK3CA and PTEN mutational status as per the protocol eligibility criteria must be done prior to the first planned administration of Study Drug.
³¹Schedule tumor biopsy. Can be performed any time after serabelisib dosing administration on day 2 (preferred) or 3 of weekly dosing (i.e., Tuesday or Wednesday if dosing started on Monday).
³²Buccal cells are collected pre- and 6 h (±30 min) post-serabelisib administration.

TABLE 3
Schedule of Activities for Subjects

| | | Intervention Period | | | | | | | | | | |
|---|---|---|---|---|---|---|---|---|---|---|---|---|
| | | | Study Drug Administration Period | | | | | | | | | |
| | | | | | | | | | | | Cycle 4 | End of |
| | | | Cycle 1 | | | Cycle 2 | | | | Cycle 3 | onward | Inter- |
| | SC | RI | W 1 | W 2 | | W 1 | W 2¹ | | W 3 | W 1 | W 1 | vention |
| | Visit (study day) | | | | | | | | | | | |
| | V1 | V2 | | | | | | | | | | (within 28 |
| | (D −28 | (D −7 | V3 | V4 | V5 | V6 | V6.1¹ | V6.2¹ | V7 | V8 | V9 | d of the |
| Procedures | to −8) | to −1) | (D 1) | (D 9) | (D 10) | (D 29) | (D 37) | (D 38) | (D 43) | (D 57) | (D 85+) | last dose) |
| Virtual visit | | X | X | X | As needed | | | | | | | |
| with dietitian | | | | | | | | | | | | |
| (meal | | | | | | | | | | | | |
| consultation, | | | | | | | | | | | | |
| training with | | | | | | | | | | | | |
| App, etc.) | | | | | | | | | | | | |
| Bring smart | | X | | | | | | | | | | |
| phone or tablet | | | | | | | | | | | | |
| to clinic visit | | | | | | | | | | | | |
| Arrive at | X | X | X | X | X | X | X | X | X | X | X | X |
| clinic fasted | | | | | | | | | | | | |
| Bring breakfast | | | X | X | X | | X | X | | | | |
| from Study ISD | | | | | | | | | | | | |
| menu to clinic | | | | | | | | | | | | |
| Bring lunch | | | X | X | | | | | | | | |
| from Study ISD | | | | | | | | | | | | |
| menu to clinic | | | | | | | | | | | | |
| Bring dispensed | | | | X | X² | X² | X² | X² | X² | X² | X² | |
| Serabelisib | | | | | | | | | | | | |
| dose to clinic | | | | | | | | | | | | |

| Fasted morning | X (daily on an ongoing basis)³ | |
|---|---|---|
| glucose and BHB | | |
| (via finger stick); | | |
| report via App | | |
| Report | X (daily on an ongoing basis) | |
| administration of | | |
| and satisfaction | | |
| with Study ISD | | |
| via App | | |
| Report body weight | X (daily on an ongoing basis) | |
| and composition | | |
| from at-home | | |
| scale via App | | |
| Report at-home | | X (daily on an ongoing basis) |
| administration | | |
| of Serabelisib | | |
| via App | | |

¹D 37 and D 38 (V6.1 and V6.2) for those in Cohort 1 only (for PK study upon dose escalation).
²Serabelisib is be self-administered on three consecutive days starting with either Monday, Tuesday, or Wednesday for the remainder of the trial.
³Fasted morning glucose and BHB measurements occur daily for two weeks upon run-in (i.e., starting on V2 and through Week 1 Cycle 1 of Serabelisib dosing) and intermittently thereafter, as directed by the Study Dietitian.

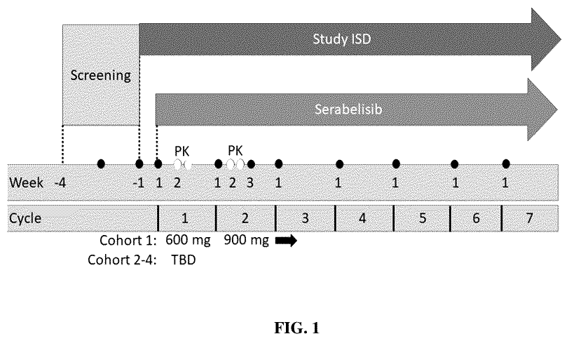

FIG. 1 depicts a flowchart of the study using serabelisib in combination with an insulin suppressing diet in subjects with cancer. The screening is run from 28 days (Week −4) to 8 days prior to the first planned administration of serabelisib. The run-in is from 7 days to 1 day prior to the first planned administration of serabelisib. Study ISD alone is administered. During Cycle 1 Week 1 and onward, serabelisib in combination with Study ISD is administered. The Study Drug is given orally on the same 3 consecutive days each week (starting with either Monday, Tuesday, or Wednesday) followed by 4 consecutive days off Study Drug. Study Drug is administered in 28-day (4-week; +3 days) cycles without a break between cycles.

Study Schedule: For all cohorts, the study comprises, or in some cases consists, of three periods: Screening and study diet Run-In (captured at Visits 1 and 2, respectively), Study Drug Cycles (Visit 3 and onward), and End of Intervention (to be conducted within 28 d of ending participation). During the Study Drug Cycles, serabelisib and the ISD are administered in 28-day (4-week; +3 days) cycles without a break between cycles. The intervention continues until subjects develop disease progression, have unacceptable toxicity, or for other protocol-specified reasons.

In general, the procedures performed during the Screening, Run-in, and Study Drug Cycles, and at the End of Intervention Visit are the same for all cohorts. Safety is evaluated by AEs, clinical laboratory tests, vital signs, ECGs, and physical examination. Efficacy is determined from tumor assessments (ORR, PFS, and OS). Pharmacokinetic and PD samples are used to estimate PK and PD parameters. TABLE 4 shows pharmacokinetic parameters used in the study.

TABLE 4
Pharmacokinetic Parameters

| Parameter | Definition |
|---|---|
| Cₘᵢₙ | The minimum observed plasma concentration |
| Cₘₐₓ | The maximum observed plasma concentration |
| tₘₐₓ | The time at which Cₘₐₓ was apparent. |
| AUC₀₋ₜ | The area under the concentration versus time curve from time zero to the |
| | sampling time at the last quantifiable concentration (Ct) at the time of the |
| | last quantifiable concentration (tₗₐₛₜ) calculated by the linear trapezoidal |
| | rule. |
| AUC_{0-∞} | The area under the concentration versus time curve estimated from time |
| | zero to infinity as the sum of the 2 areas: AUC₀₋ₜ and AUCₑₓₜᵣₐₚ, where |
| | AUCₑₓₜᵣₐₚ is calculated as Ct/λz. |
| AUCₑₓₜᵣₐₚ | The area under the plasma concentration versus time curve extrapolated |
| | from time t to infinity as a percentage of total AUC. |
| λz | The apparent terminal rate constant, estimated using the negative slope of |
| | the least square regression analysis of the log concentration versus time |
| | data for the terminal linear portion of the curve. |
| t_{1/2} | The apparent terminal half-life, calculated from Ln 2/λz. |
| CL | The systemic clearance calculated as: Dose/AUC_{0-∞}. |
| V_{d} | The apparent volume of distribution, calculated as the amount of drug in |
| | the body divided by the concentration of drug in the blood/plasma. |
| Vₛₛ | The apparent volume of distribution at steady state. |

A written Informed Consent Form (ICF) is obtained before any study-specific screening evaluations are performed and is documented in the subject's medical chart. After obtaining the ICF, subjects are screened for enrollment at Visit 1 (Study days −28 to −8). The screening procedures include evidence by a fresh tumor biopsy or archival tissue confirming mutational status for PIK3CA and PTEN. If mutational status is unknown or not performed within the past 6 months, analysis of archival or fresh tumor tissue is completed prior to screening. Other screening procedures include collection of circulating tumor markers (e.g., CEA, CA 125, CA 19-9; selected by the treating physician), a physical examination, medical history, safety tests (e.g., lab tests, ECG), and tumor imaging. Eligible subjects are enrolled to participate in the initial cohort until RP2D is determined, and in the appropriate expansion cohort thereafter.

The following procedures and fasting laboratory assessments are performed at Visit 1, between 28 days and 8 days prior to the first planned administration of Study Drug (Cycle 1, Visit 3, Study Day 1):

Procedures: Signed ICF; Review of inclusion/exclusion criteria; Medical history (including demographics); Complete physical examination; Height, weight, ECOG PS; Vital signs (heart rate, respiratory rate, temperature, and blood pressure); 12-lead ECG; Fasting Laboratory assessments (not required if performed within 14 days prior to screening); Serum pregnancy test for females of childbearing potential only; Review of prior 30 d and concomitant medications; Circulating tumor marker assessment; Available archival tumor tissues or fresh tumor tissue are sent to the local laboratory for PD analysis.

Laboratory assessments:
Hematology: complete blood count (CBC) with differential (percent and absolute), hemoglobin (Hb), mean corpuscular volume (MCV), mean corpuscular hemoglobin concentration (MCHC), mean corpuscular hemoglobin (MCH), red cell distribution width (RDW), and platelet count
Chemistry: blood urea nitrogen (BUN), creatinine, alanine aminotransferase (ALT), aspartate transaminase (AST), alkaline phosphatase (ALP), total bilirubin, direct bilirubin, lactate dehydrogenase (LDH), total protein, sodium, potassium, chloride, bicarbonate, calcium, phosphate, and magnesium
Metabolic: blood glucose, insulin, triglycerides, total cholesterol, LDL-direct, HDL, C-peptide, beta-hydroxybutyrate (BHB), and non-esterified fatty acids (NEFA)
   Including glycated hemoglobin (HbA1c; not required if performed within 14 d prior to screening) and glycated albumin
Urinalysis: dipstick protein, glucose, ketones, and blood; bilirubin, urobilinogen, pH, specific gravity, nitrite, and leukocyte esterase. A microscopic examination is performed, if necessary
Circulating tumor markers: selected by the treating physician (such as CEA, CA 19-9, and CA 125). Marker selection must remain consistent throughout study participation.

At Visit 2 (Study Days-7), subjects come to the clinic for placement of the continuous glucose monitor (CGM) and discussion with the study dietitian. Subjects are provided with a backup CGM sensor and education on how to remove sensor, return sensor to clinic, and replace a sensor. The second CGM serves as a backup to be kept for the remainder of study participation. Should the backup sensor be used, another backup sensor is provided. The sensor remains placed until removed and replaced with a new sensor on Visit 3, Day 1. The first delivery of the Study ISD to the subject is delivered the day before or the day of Visit 2. The Study ISD is initiated the day following Visit 2 on Day-6. Subjects bring their smart-phone or tablet to the visit.

The following evaluations and procedures are performed: Vital signs (heart rate, respiratory rate, temperature, and blood pressure); Body Weight; CGM initiation (sensor placed according to Prescribing Information); Fasting laboratory assessments; Buccal cell collection; Assessment of AEs (non-intervention-emergent AEs); Review of prior (past 30 d) and concomitant medications; Introduction and training on the Study ISD and consult with the study dietitian; Training on App utilization.

Laboratory assessments: Metabolic assessments including blood glucose, insulin, triglycerides, total cholesterol, LDL-direct, HDL, C-peptide, beta-hydroxybutyrate (BHB), and non-esterified fatty acids (NEFA).

Virtual visits through the App from study dietitian are used to check in on App use and how data entry is going, issues or questions with meals, and any other questions.

The Study Drug is dispensed according to the schedule of events for administration at home in between clinic visits. The Study Drug Administration Period begins at the time of serabelisib initiation and ends with the completion of the End of Intervention (EOI) visit. Subjects receive the Study Drug as continuous 28±3 day cycles. Subjects undergo safety and efficacy assessments. Subjects continue to receive the Study Drug and Study ISD until disease progression, development of unacceptable toxicity, or withdrawal of consent.

At Visit 3 (Cycle 1 Week 1 Day 1), subjects come to the clinic to begin serabelisib administration. Subjects must arrive at the clinic fasted (water consumption encouraged but no food for at least 6 hours prior and must bring breakfast and lunch from their Study ISD menu). During the visit: CGM data are downloaded and a new sensor is applied at least 1 h prior to serabelisib dosing; serabelisib administration is initiated (600 mg for Cohort 1, and RP2D for Cohorts 2, 3, and 4) 15-20 min after finishing the Study ISD breakfast; serabelisib is dispensed and subjects continue with dosing on the same three consecutive days (starting with either Monday, Tuesday, or Wednesday) for the remainder of the trial. Subjects receive their serabelisib on a monthly basis.

Procedures prior to breakfast include: Vital signs (heart rate, respiratory rate, temperature, and blood pressure): up to 60 min prior to dosing; Body Weight; 12-lead ECG: in triplicate after 3 min rest in sitting position up to 60 min prior to dosing; Fasting Blood Collection for Laboratory Assessments; CGM download and replacement (remove sensor that was placed at Visit 2 and replace with a new sensor at least 1 h prior to taking Study ISD).

ISD breakfast and administration of serabelisib includes: Eat Study ISD breakfast; Administer serabelisib within 15-20 min after finishing breakfast. Procedures after breakfast and serabelisib include: Vital signs (heart rate, respiratory rate, temperature, and blood pressure): post-dose at 6 h (±30 min); 12-lead ECG: in triplicate after 3 min rest in sitting position post-dose at 6 h (±30 min); Assessment of AEs. Laboratory assessments from visit 2 are repeated. Additional procedures include dispensing the Study Drug (serabelisib) and providing dosing instructions. A virtual visit from the study dietitian is conducted to check in on App use and how data entry is going, how meals are working out, and any other questions that occur on study day 4.

At Visit 4 (Cycle 1, Week 2, Day 9), subjects arrive for a safety assessment and PK/PD testing. Visit occurs on the second day of serabelisib dosing and occurs no later than Thursday (to ensure the lab can receive PK samples). Subjects arrive at the clinic fasted (water consumption encouraged but no food for at least 6 hours prior). Subjects bring breakfast and lunch from their Study ISD menu and their serabelisib dose with them to the clinic.

During the visit: Serial blood samples are collected for PK; Buccal cells are collected pre- and post-serabelisib administration for PD; the subject eats the Study ISD breakfast and consumes the daily serabelisib dose; CGM placed at Visit 3 remains in place.

Procedures prior to breakfast include: Vital signs (heart rate, respiratory rate, temperature, and blood pressure): up to 60 min prior to dosing; Body Weight; 12-lead ECG: in triplicate after 3 min rest in sitting position up to 60 min prior to dosing; Fasting blood collection for Laboratory Assessments; Buccal cell collection: pre-dose (see protocol in laboratory manual); Assessment of AEs; Review of concomitant medications.

ISD breakfast and administration of serabelisib is the same as Visit 3. Procedures after breakfast and serabelisib include: Vital signs (heart rate, respiratory rate, temperature, and blood pressure) 60 min (+30 min) following dosing; 12-lead ECG: in triplicate after 3 min rest in sitting position 6 h (±30 min) following dosing; Blood collection for glucose, serum insulin, C-peptide, BHB, NEFA, and Serabelisib PK: post-dose at 1.5 h (±15 min), 3 h (±30 min), 6 h (±30 min), 9 h (±1 h), and 12 h (±1 h); Consultation with study dietitian (may be virtual); Eat Study ISD lunch (3-4 h after dose); Buccal cell collection: post-dose at 6 h (±30 min); Eat Study ISD dinner (8-9 h after dose); Schedule the subject to come back to the clinic at 24 h after the administration of Study Drug to finish PK assessment; Assessment of AEs, if any to report after dosing.

Laboratory assessments include hematology, chemistry, metabolic, and urinalysis measurements of visit 3, as well as a serabelisib PK analysis.

At Visit 5 (Cycle 1 Week 2 Day 10), subjects come to the clinic to finish PK assessment. Subjects arrive at the clinic fasting (water consumption encouraged but no food for at least 6 hours prior) and prior to serabelisib dose. Subjects bring breakfast from their Study ISD menu and bring their serabelisib dose. During the visit: a blood sample is collected for metabolic testing and PK; and the subject can then consume breakfast and consume their daily serabelisib dose.

The following evaluations and procedures are performed: Vital signs (heart rate, respiratory rate, temperature, and blood pressure); Body Weight; Fasting blood collection (collected 24 h [±2 h] after the Visit 4 dose); Metabolic: glucose, insulin, C-peptide, BHB, and NEFA; Serabelisib PK analysis; Assessment of AEs (non-intervention-emergent AEs); CGM sensor placed at Visit 3 is removed and data are downloaded. Subjects are provided with two unopened CGM sensors; one is placed in the clinic at least 1 h prior to taking the Study Drug and the other is sent home with the subject along with education on how to remove the sensor, store the sensor for later return to the clinic, and replace the sensor with new one on Day 24. Subjects are reminded via phone call on Day 23. Serabelisib is taken within 15-20 min of finishing Study ISD breakfast.

Starting in Week 2, V5, virtual visits from the study dietitian are only scheduled on an ad hoc basis if a subject's App data around meal compliance, drug compliance, ketone levels, or glucose levels are in question or if the subject has a question. The study dietitian schedules a meeting with the subject. The study dietitian is available to the subject, upon request through the App. All insights regarding subject information are communicated to the study team. The Study team is notified regarding any safety issues or recommendations made.

At Visit 6 (Cycle 2 Week 1 Day 29), subjects arrive for a safety assessment. Subjects arrive at the clinic fasted (water consumption encouraged but no food for at least 6 hours prior). Subjects can bring breakfast from their Study ISD menu and their serabelisib dose. Subjects in Cohort 1 serabelisib dosing can be increased from 600 mg to 900 mg for Cycle 2.

The following evaluations and procedures are performed: Vital signs (heart rate, respiratory rate, temperature, and blood pressure); Body Weight; Fasting blood collection for Laboratory Assessments; Assessment of AEs (non-intervention-emergent AEs); Subjects return the first sensor and remove the second sensor provided at Visit 5. Data from both sensors is to be downloaded. A new sensor is placed in the clinic at least 1 h prior to taking the Study Drug. Schedule tumor biopsy. Must be performed 6 h (±1 h) after dosing on day 2 or 3 of weekly dosing (i.e., Tuesday or Wednesday). Tumor tissue is sent to the local laboratory for PD analysis. Laboratory Assessments from visit 1 are repeated. After the fasting labs, the subject can then consume breakfast and consume their daily serabelisib dose 15-20 min following breakfast completion.

At Visit 6.1 (Cycle 2, Week 2, Day 37), subjects in Cohort 1 only arrive for a safety assessment and PK/PD testing after serabelisib dose modification to 900 mg. Visit 6.1 occurs on the second day of weekly serabelisib dosing and occurs no later than Thursday to ensure the lab can receive PK samples. Subjects arrive at the clinic fasted (water consumption encouraged but no food for at least 6 hours prior). Subjects bring breakfast and lunch from their Study ISD menu and their serabelisib dose with them to the clinic.

During the visit: Serial blood samples are collected for PK; Buccal cells are collected pre- and post-serabelisib administration for PD; the subject eats the Study ISD breakfast and consumes the daily serabelisib dose; CGM sensor placed at Visit 6 remains in place.

Procedures prior to breakfast include those listed for Visit 4. ISD breakfast and administration of serabelisib are the same as Visit 3. Procedures after breakfast and serabelisib include: Vital signs (heart rate, respiratory rate, temperature, and blood pressure) 60 min (±30 min) following dosing; 12-lead ECG: in triplicate after 3 min rest in sitting position 6 h (±30 min) following dosing; Blood collection for glucose, serum insulin, C-peptide, BHB, NEFA, and Serabelisib PK: post-dose at 1.5 h (±15 min), 3 h (±30 min), 6 h (±30 min), 9 h (±1 h), and 12 h (±1 h); Eat Study ISD lunch (3-4 h after dose); Buccal cell collection: post-dose at 6 h (±30 min); Eat Study ISD dinner (8-9 h after dose); Schedule the subject to come back to the clinic at 24 h after the administration of Study Drug to finish PK assessment; Assessment of AEs, if any to report after dosing. Laboratory assessments include assessments listed in visit 3.

At Visit 6.2 (Cycle 2 Week 2 Day 38), subjects in Cohort 1 only come to the clinic to finish PK assessment. Subjects arrive at the clinic fasting (water consumption encouraged but no food for at least 6 prior) and prior to serabelisib dose. Subjects bring breakfast from their Study ISD menu and bring their serabelisib dose. During the visit: a blood sample is collected for metabolic testing and PK; the subject can then consume breakfast and consume their daily serabelisib dose; the CGM placed at Visit 6 remains in place.

The following evaluations and procedures are performed: Vital signs (heart rate, respiratory rate, temperature, and blood pressure); Body Weight; Fasting blood collection (collected 24 h [+2 h] after the Visit 4 dose); Metabolic: glucose, insulin, C-peptide, BHB, and NEFA; Serabelisib PK analysis; Assessment of AEs (non-intervention-emergent AEs); Take serabelisib within 15-20 min of finishing Study ISD breakfast.

A Safety Review Committee (SRC) is convened after the last subject enrolled in a Cohort 1 has completed Cycle 2 with Study ISD and serabelisib to determine safety and establish a dosing regimen (RP2D) for expansion cohorts. Safety is determined from metabolic panel results (particularly those related to liver function) and IRAEs.

At Visit 7 (Cycle 2 Week 3 Day 43), subjects arrive for a safety assessment. Subjects arrive to the clinic fasted (water consumption encouraged but no food for at least 6 hours prior). Subjects can bring breakfast from their Study ISD menu and their serabelisib dose.

The following evaluations and procedures are performed: Vital signs (heart rate, respiratory rate, temperature, and blood pressure); Body Weight; Fasting blood collection for Laboratory Assessments; Assessment of AEs (non-intervention-emergent AEs); CGM sensor placed at Visit 6 is removed and data is downloaded. A new sensor is placed in the clinic at least 1 h prior to taking the Study Drug.

Laboratory Assessments include hematology, chemistry, metabolic, and urinalysis assessments of visit 3. After the fasting labs, the subject can then consume breakfast and consume their daily serabelisib dose 15-20 min following breakfast completion. A call to the subject as a reminder to replace CGM sensor occurs on study day 51.

At Visit 8 (Cycle 3 Week 1 Day 57), subjects arrive for a safety assessment. Subjects arrive at the clinic fasted (water consumption encouraged but no food for at least 6 hours prior). Subjects may bring breakfast from their Study ISD menu and their serabelisib dose.

The following evaluations and procedures are performed: Vital signs (heart rate, respiratory rate, temperature, and blood pressure); Body Weight; Fasting blood collection for Laboratory Assessments; Assessment of AEs (non-intervention-emergent AEs); Schedule tumor imaging; Remove CGM and download data. CGM data are not collected for the remainder of study participation.

Laboratory Assessments include hematology, chemistry, metabolic, urinalysis, and circulating tumor marker assessments of visit 1. After the fasting labs, the subject can then consume breakfast and consume their daily serabelisib dose 15-20 min following breakfast completion.

Subjects arrive on the 1st day of week 1 for each cycle for a safety assessment. Subjects arrive at the clinic fasting (water consumption encouraged but no food for at least 6 hours prior). Subjects may bring breakfast from their Study ISD menu and their serabelisib dose. The following evaluations and procedures are performed: Vital signs (heart rate, respiratory rate, temperature, and blood pressure); Body Weight; Fasting blood collection for Laboratory Assessments; Assessment of AEs (non-intervention-emergent AEs); Tumor imaging: after completion of each even-numbered cycle.

Laboratory Assessments for hematology, chemistry, metabolic, urinalysis, and circulating tumor marker assessments of visit 1 are used. Marker selection remains consistent throughout study participation, and is collected upon the first visit of each subsequent Study Drug cycle for the first 6 months of participation, and then every three months thereafter. After the fasting labs, the subject can then consume breakfast and consume their daily serabelisib dose 15-20 min following breakfast completion.

Subjects continue the study in 28-day (4-week; ±3 days) cycles without a break between cycles. Intervention continues until subjects develop disease progression, have unacceptable toxicity, or for other protocol-specified reasons. An End of Intervention Visit is performed within 28 days after the last administration of Study ISD and drug.

The following evaluations and procedures are performed within 28 days of the last administration of Study Drug: Vital signs (heart rate, respiratory rate, temperature, and blood pressure); Body Weight; ECOG PS; Fasting blood collection for Laboratory Assessments; Assessment of AEs (non-intervention-emergent AEs); Study drug reconciliation.

Laboratory Assessments include: Hematology: CBC with differential (percent and absolute), Hb, MCV, MCHC, MCH, RDW, and platelet count; Chemistry: BUN, creatinine, ALT, AST, ALP, total bilirubin, direct bilirubin, LDH, total protein, sodium, potassium, chloride, bicarbonate, calcium, phosphate, and magnesium; Metabolic: blood glucose, insulin, triglycerides, total cholesterol, LDL-direct, HDL, C-peptide, BHB, NEFA, hemoglobin A1C, and glycated albumin; Urinalysis: dipstick protein, glucose, ketones, and blood; bilirubin, urobilinogen, pH, specific gravity, nitrite, and leukocyte esterase. A microscopic examination is performed, if necessary; and Circulating tumor markers: selected by the treating physician (such as CEA, CA 19-9, and CA 125).

Inclusion and Exclusion Criteria: Subjects are included only if they meet all of the following criteria:
1. Able to provide written informed consent.
2. Age ≥18 at Visit 1 (screening).
3. Histologically or cytologically confirmed recurrent solid tumors.
   a. For cohort expansion:
      i. Cohort 2: Histological documentation of adenocarcinoma of the colon or rectum.
      ii. Cohort 3: Recurrent or persistent endometrial adenocarcinoma with the following histologic epithelial cell types are eligible: endometrioid adenocarcinoma, serous adenocarcinoma, undifferentiated carcinoma, clear cell adenocarcinoma, mixed epithelial carcinoma, adenocarcinoma not otherwise specified (N.O.S.), mucinous adenocarcinoma, squamous cell carcinoma, transitional cell carcinoma, and carcinosarcoma.
      iii. Cohort 4: Histologically (primary tumor with ≥50% clear cell histomorphology) or cytologically confirmed ovarian clear cell or endometrioid carcinoma.
4. Tumor must harbor an activating mutation in the PIK3CA gene with or without PTEN loss, either previously documented or determined during screening.
5. Fresh or archival tumor biopsy with sufficient material to be sent to the designated laboratory for PD analyses. If only slides are available, then a minimum of 15 slides is required and the tumor tissue must have been obtained within 6 months of screening.
6. Dose Modification cohort: failed, were intolerant of, or ineligible for no more than 3 prior lines of systemic therapy (LOT) for advanced/metastatic disease or refused standard-of-care therapy (not counting neoadjuvant/adjuvant therapy).
7. Expansion cohorts: failed, were intolerant of, ineligible for, or have refused standard-of-care therapy for advanced/metastatic disease (not counting neoadjuvant/adjuvant therapy) and
   a. For endometrial cancer subjects: Have no more than 2 prior LOT. Concurrent hormonal therapy is NOT allowed.
   b. For ovarian clear cell or endometrioid carcinoma subjects: Have no more than X prior LOT and must have platinum-resistant (recurrence within 6 months of platinum treatment), or platinum refractory disease (recurrence during platinum treatment).
   c. For colorectal cancer subjects: Have no more than 2 prior LOT for metastatic disease.
8. Life expectancy of at least 3 months.
9. Documented radiographic progression since the last treatment was administered with at least one measurable lesion (as defined by RECIST 1.1).
10. Eastern Cooperative Oncology Group performance status (ECOG PS) of 0-1.
11. Adequate organ function
   a. Absolute neutrophil count (ANC) ≥1.5×10⁹/L, platelet count ≥75×10⁹/L, and hemoglobin >9 gm/dL (may be transfused to reach this Hb level unless due to blood loss).
   b. Transaminases (aspartate aminotransferase [AST] and alanine aminotransferase [ALT])≤2.5× upper limit of normal (ULN) (<5×ULN if liver metastases are present), and total bilirubin≤1.5×ULN (<3×ULN if subject has Gilbert Syndrome).
   c. Renal: calculated or measured creatinine clearance >50 mL/min/1.73 m² (body surface area-adjusted Cockcroft-Gault or other appropriate formula).
12. Ability to take oral medication, be willing to adhere to study procedures and Study Drug administration, and receive, consume, and comply with Study ISD.
13. For women of childbearing potential, a negative serum pregnancy test collected at screening (V1) and negative urine pregnancy test collected at baseline (V2) and use of physician-approved method of birth control from 30 days before the planned first administration of Study Drug to 90 days following the last administration of Study Drug.
14. Male subjects must be surgically sterile or must agree to use physician-approved contraception during the study and for 90 days following the last administration of Study Drug.

Subjects are excluded for any of the following reasons:
1. Diagnosis of primary brain tumor.
2. Leptomeningeal disease and symptomatic or untreated brain metastases. Subjects with treated (surgically excised or irradiated) and stable brain metastases are eligible, assuming adequate recovery from treatment to Grade 1 or less, the treatment was at least 28 days prior to planned first day of Study Drug, administration and baseline brain computed tomography (CT) with contrast or magnetic resonance imaging (MRI) within 14 days of initiation of Study Drug, is negative for new or worsening brain metastases.
3. Diagnosis of another malignancy within the past 2 years (excluding a history of carcinoma in situ of the cervix, superficial non-melanoma skin cancer, or superficial bladder cancer that has been adequately treated, or stage 1 prostate cancer that does not require treatment or requires only treatment with luteinizing hormone-releasing hormone agonists or antagonists if initiated at least 90 days prior to the first planned day of Study Drug administration).
4. Is less than 21 days from therapeutic radiation or chemotherapy prior to the first planned day Study Drug administration and has not recovered to Grade≤1 (National Cancer Institute [NCI] Common Terminology Criteria for Adverse Events [CTCAE] v5.0, Appendix 5) from all clinically significant toxicities related to prior therapies.
5. For subjects receiving nitrosoureas or mitomycin C, the subject is <6 weeks from last dose. For monoclonal antibody therapy, the subject is <one half-life or <4 weeks from last dose, whichever is shorter.
6. Chronic, systemically administered glucocorticoids in doses equivalent to >5 mg prednisone daily. Topical, inhalational, ophthalmic, intraarticular, and intranasal glucocorticoids are permitted. Isolated or intermittent use of systemically administered glucocorticoids to treat complications of malignancy, use as a premedication, or as a one-time prep for an imaging procedure is permitted. If subject was on >5 mg prednisone/day equivalent, last dose must have been at least 7 days prior to the first planned dose of the Study Drug. Replacement corticosteroids for adrenal insufficiency are permitted.
7. Diabetes mellitus requiring insulin or insulin secretagogue therapy.
8. Poorly controlled diabetes mellitus defined as glycosylated hemoglobin A1c (HbA1c)>7.5% or fasting blood sugar >160 mg/dL.
9. Have a secondary malignancy requiring therapy or are unstable without therapy.
10. Known impaired cardiac function or clinically significant cardiac disease such as ventricular arrhythmia requiring therapy, congestive heart failure (New York Heart Association [NYHA] Class III or IV), and/or uncontrolled hypertension (defined as systolic blood pressure >150 mmHg and/or diastolic blood pressure >100 mmHg on antihypertensive medications).
11. Myocardial infarction, cardiac stent placement, or unstable angina within 6 months before the planned first administration of Study Drug.
12. Have clinically significant peripheral vascular disease.
13. Manifestations of malabsorption due to prior GI surgery, GI disease, or for an unknown reason that could alter the absorption of oral medications or Study ISD.
14. Other clinically significant comorbidities, such as uncontrolled pulmonary disease, active central nervous system disease, active infection (any infection requiring systemic therapy), non-healing wounds or injuries, or any other condition that could compromise the subject's participation.
15. Pregnant (positive serum pregnancy test), planning to become pregnant during or within 90 days after the last dose of Study Drug, or breastfeeding/planning to breastfeed.
16. Have taken strong CYP3A4 inducers/inhibitors within 7 days before the first administration of serabelisib or have conditions that require the concomitant use of CYP3A4 inducers/inhibitors.
17. Untreated or poorly controlled, gastro-esophageal reflux disease.
18. Have taken histamine-H2 receptor antagonists within 24 hours before the first administration of serabelisib.
19. Require or have taken proton-pump inhibitors (PPI) within 7 days or 5 half-lives (whichever is the shorter duration) before the first administration of serabelisib.
20. Have taken neutralizing antacids within 4 hours before the first administration of serabelisib.
21. Known to be positive for Human Immunodeficiency Virus (HIV), Hepatitis B, or Hepatitis C.
22. Known allergic reactions to excipients of the Study Drug (list needed: X, Y, Z).
23. Severe, uncontrolled gout.
24. A body mass index (BMI)<20 kg/m², or serious or refractive cachexia or anorexia that, in the investigator's opinion, realistically prohibits subjects from having energy or appetite sufficient to reliably engage in a strict medical food regimen for an extended time.
25. Any condition that renders the subject unable to satisfactorily chew, swallow, digest, or tolerate (i.e., persistent diarrhea) the majority of foods and liquids of the Study ISD.
26. History of severe nephrolithiasis requiring urologic intervention.
27. Participation in a diet (Atkins, Weight Watchers, Best Life, Nutrisystem, South Beach, Jenny Craig, Paleo Diet, Zone etc.) or weight loss plan within 10 days prior to the planned first administration of Study Drug.
28. Severe constipation or condition where exacerbation of constipation is not advisable (e.g., small bowel obstruction history).
29. History of anaphylaxis from food allergy or other disease state requiring avoidance of a particular food, such as celiac disease.
30. No daily access to a computer, tablet, or phone with internet access.
31. History of diagnosed eating disorder.
32. Unwilling to take a non-vegan or non-vegetarian diet.

Dose Modification and Determination (Cohort 1): A dose-modification scheme (Cohort 1; n=8) is employed to determine the optimal, safe dosing regimen for the combination of serabelisib and the Study ISD in the cohort expansion. The starting dose of serabelisib is 600 mg once daily (QD) on the same three consecutive days of each week (which must start on either a Monday, Tuesday, or Wednesday), which is less than the previously recommended Phase 2 dose (RP2D) of 900 mg QD administered on three consecutive days each week. Pharmacokinetics (PK) are assessed following one week of the 600 mg dosing (PK assessments occur in cycle 1, week 2). Following one 28 d cycle of 600 mg, and if 600 mg is tolerable, serabelisib dosing is escalated to 900 mg, and PK is again assessed following one week of the 900 mg dosing (PK assessment occur in cycle 2, week 2).

The starting dose of 600 mg PO QD is selected because of the MTD at 900 mg seen in the Phase 1a study and the potential for higher exposure of drug when given with meals. In addition, the intermittent schedule given in the same 3 consecutive days out of each week (e.g., M/Tu/W, Tu/W/Th, W/Th/F) is used based on the lower percentage of patients with elevated liver enzymes.

Dose modification, with an enrollment of 8 subjects, is designed to determine the serabelisib recommended dose (RP2D) in combination with Study ISD to be employed in the expansion cohorts (Cohorts 2, 3, and 4). One cohort (Cohort 1) with different tumor types, PIK3CA mutations (mandatory) with or without PTEN loss (no restrictions on proportion of subjects with PTEN loss) is used to assess the safety and tolerability of the combination of Study ISD co-administered with serabelisib at 600 mg for the first cycle (28 d) followed by 900 mg upon the start of the second cycle; doses are administered on 3 consecutive days of every week beginning with either a Monday, Tuesday, or Wednesday.

TABLE 5
Study Intervention

| | | Study Intervention | | |
|---|---|---|---|---|
| | Approximate | | Insulin | |
| | Number of | Serabelisib | Suppressing | |
| Cohort | Subjects | QD 3 d/Week | Diet¹ | Indications |

Dose modification: 8 subjects

| | | | | |
|---|---|---|---|---|
| 1 | 8 | 600 mg, escalated to | Yes | Solid Tumor |
| | | 900 mg after one cycle | | |

Cohort expansion: 42 subjects

| | | | | |
|---|---|---|---|---|
| 2 | 14 | (RP2D according to | Yes | CRC |
| | | Cohort 1) | | |
| 3 | 14 | (RP2D according to | Yes | EC |
| | | Cohort 1) | | |
| 4 | 14 | (RP2D according to | Yes | OC |
| | | Cohort 1) | | |

Dose modification to determine RP2D is conducted in Cohort 1 (subjects with mixed advanced cancers with PIK3CA mutations with or without PTEN loss. Safety data for cohort 1 after the last patient completes cycle 2 are evaluated by the SRC to determine the RP2D for expansion cohorts.

TABLE 6
Description of Dose Modification Cohort (Cohort 1)

| | | | Intervention | |
|---|---|---|---|---|
| | Approximate | | Serabelisib | |
| | Number of | Cancer/Mutational | PO QD | Study |
| Cohort | Subjects | Status | 3 d/wk | ISD¹ |
| 1 | 8 | PIK3CA-mutated | 600 mg, | Yes |
| | | with or without | escalated | |
| | | PTEN loss solid | to 900 mg | |
| | | tumors | after one | |
| | | | cycle | |

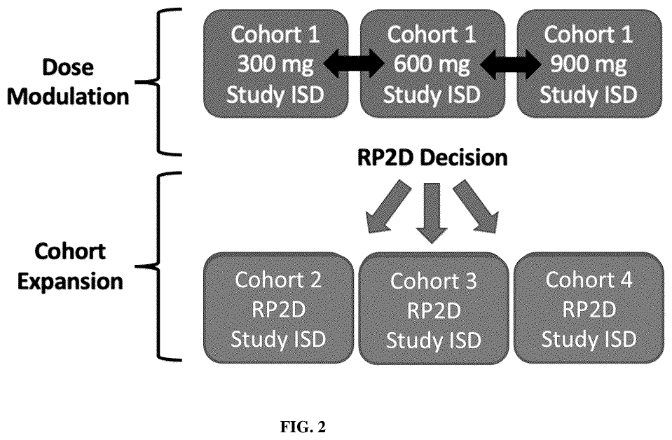

Cohort Expansion (Cohorts 2, 3, and 4): Cohort expansion includes three groups of subjects with advanced cancer to evaluate and confirm the safety of the combination of serabelisib and the Study ISD RP2D from dose modification (Cohort 1) results and to evaluate efficacy. The expansion cohorts include subjects with solid tumors that have failed standard-of-care (SOC) therapy and possess PIK3CA-activating mutations with or without PTEN loss: colorectal cancer (Cohort 2), endometrial cancer (Cohort 3), and ovarian endometrioid or clear cell carcinoma (Cohort 4). FIG. 2 depicts a representation of the study cohorts of the clinical trial using serabelisib in combination with an insulin suppressing diet in subjects with cancer.

Cohort expansion, with a planned enrollment of 42 subjects, comprises, or in some cases consists of, three parallel groups of subjects based on tumor type and mutational status. Cohort 2 (n=14): PIK3CA-mutated (mandatory) with or without PTEN loss (up to 6 of 14 subjects permitted) colorectal cancer. Cohort 3 (n=14): PIK3CA-mutated (mandatory) with or without PTEN loss (up to 6 of 14 subjects permitted) endometrial cancer. Cohort 4 (n=14): PIK3CA-mutated (mandatory) with or without PTEN loss (up to 6 of 14 subjects permitted) clear cell/endometrioid ovarian cancer. Subjects receive the RP2D regimen determined from dose modification (Cohort 1) plus the Study ISD.

With cohort expansion, subjects are assigned in parallel to expansion cohorts based on tumor type and mutational status. TABLE 7 describes expansion cohorts of the study.

TABLE 7
Description of Expansion Cohorts (Cohort 2, 3, and 4)

| | | | Intervention | |
|---|---|---|---|---|
| | Approximate | | Serabelisib | |
| | Number of | | PO QD | Study |
| Cohort | Subjects | Cancer/Mutational Status | 3 d/wk | ISD |
| 2 | 14 | PIK3CA-mutated with or | RP2D | Yes |
| | | without PTEN loss | | |
| | | colorectal cancer | | |
| 3 | 14 | PIK3CA-mutated with or | RP2D | Yes |
| | | without PTEN loss | | |
| | | endometrial cancer | | |
| 4 | 14 | PIK3CA-mutated with or | RP2D | Yes |
| | | without PTEN loss ovarian | | |
| | | endometrioid or clear cell | | |
| | | carcinoma | | |

Controlling Adverse Effects: If the starting serabelisib dose of 600 mg QD is not tolerated, then the subject is de-escalated to 300 mg. If 300 mg QD for three days per week is not tolerated, then the subject is discontinued because a lower dose is not anticipated to be biologically active. TABLE 8 shows serabelisib dose reduction guidelines.

TABLE 8
Serabelisib Dose Reduction Guidelines

| Serabelisib dose level | Dose and schedule |
|---|---|
| Starting dose | 600 mg once daily 3CDW¹ |
| Dose Increase² | 900 mg once daily 3CDW |
| First dose reduction | 600 mg once daily 3CDW |
| Second dose reduction | 300 mg once daily 3CDW |
| First dose reduction from 600 mg⁴ | 300 mg once daily 3CDW³ |

¹All dose levels (300 mg, 600 mg, 900 mg) are given daily on a 3CDW (three consecutive days per week) schedule.
²Dose cannot be increased above 900 mg; if subject is at 300 mg, dose cannot be increased more than 300 mg at a time (i.e., subject cannot go from 300 mg to 900 mg at once but must proceed by 300 mg increments to a top dose of 900 mg).
³If further dose reduction below 300 mg is required, discontinue serabelisib.
⁴If subject cannot tolerate the starting dose of 600 mg.

Nausea, vomiting, and diarrhea are treated with concomitant medications if warranted. If symptoms persist despite optimal treatment, then, depending on the severity of the AE, the Study Drug is reduced or stopped until symptoms have improved. TABLE 9 shows recommended dose modifications and management for nausea while TABLE 10 shows recommended dose modifications and management for vomiting. TABLE 11 shows recommended dose modifications and management for diarrhea.

The Eastern Cooperative Oncology Group Scale of Performance Status (ECOG PS) is widely used to quantify the functional status of cancer patients, and is an important factor determining prognosis in a number of malignant conditions. The PS describes the status of symptoms and functions with respect to ambulatory status and need for care. PS Grade 0 is described as fully active and able to carry on all pre-disease performance without restriction. PS Grade 1 is described as restricted in physically strenuous activity but ambulatory and able to carry out work of a light or sedentary nature. PS Grade 2 is described as ambulatory and capable of all self-care but unable to carry out any work activities (i.e., up and about more than 50% of waking hours). PS Grade 3 is described as capable of only limited self-care and is confined to a bed or chair more than 50% of waking hours. PS Grade 4 is described as completely disabled, cannot carry on any self-care, and totally confined to bed or chair. PS Grade 5 is dead.

TABLE 9
Dose Modification and Management for Nausea

| Grade | Recommendation |
|---|---|
| Grade 1 | No serabelisib dose adjustment required. Provide medical |
| Loss of appetite without | therapy as needed and monitor as clinically indicated. |
| alteration in eating habits | |
| Grade 2 | Initiate or intensify appropriate medical therapy and monitor as |
| Oral intake decreased | clinically indicated. Stop serabelisib dosing until recovery to |
| without significant | Grade ≤1, then resume serabelisib at same dose level. After |
| weight loss, dehydration, | resumption, if recurs at > Grade 1, stop serabelisib until ≤Grade |
| or malnutrition | 1, and resume at lower dose level. If subject is already at 300 mg |
| | dose level, monitor for no more than 14 days and, if ≥Grade 2 |
| | with no resolution despite medical management, discontinue |
| | serabelisib. |
| Grade 3 (No Grade 4 | Initiate or intensify appropriate medical therapy and monitor as |
| category) | clinically indicated. Stop serabelisib dosing until recovery to |
| Inadequate oral caloric | Grade ≤1, then resume serabelisib at the next lower dose level. |
| or fluid intake; tube | After resumption, if recurs at Grade >1, stop serabelisib and |
| feeding, TPN, or | resume at next lower dose level. If subject is already at 300 mg, |
| hospitalization indicated | resume 300 mg dose level, but if there is recurrent nausea of |
| | ≥Grade 2, stop serabelisib permanently. |

TABLE 10
Dose Modification and Management for Vomiting

| Grade | Recommendation |
|---|---|
| Grade 1 | No serabelisib dose adjustment required. Provide |
| 1 episode/24 hrs | medical therapy as needed and monitor as clinically |
| | indicated. |
| Grade 2 | Initiate or intensify appropriate medical therapy |
| 2-5 episodes/24 hrs; | and monitor as clinically indicated. Stop serabelisib |
| outpatient IV | dosing until recovery to Grade ≤1, then resume |
| hydration; medical | serabelisib at same dose level. After resumption, if |
| intervention | recurs at >Grade 1, stop serabelisib until ≤Grade 1, |
| indicated | and resume at lower dose level. If subject is already |
| | at 300 mg dose level, monitor for no more than 14 |
| | days and, if ≥Grade 2 with no resolution despite |
| | medical management, discontinue serabelisib. |
| Grade 3 | Initiate or intensify appropriate medical therapy and |
| 6-10 episodes/24 | monitor as clinically indicated. Stop serabelisib |
| hrs; tube feeding, | dosing until recovery to Grade ≤1, then resume |
| TPN, or | serabelisib at the next lower dose level. After |
| hospitalization | resumption, if recurs at Grade >1, stop serabelisib |
| indicated | and resume at next lower dose level. If subject is |
| | already at 300 mg, resume 300 mg dose level, but if |
| | there is recurrent vomiting of ≥Grade 2, stop |
| | serabelisib permanently. |
| Grade 4 | Initiate or intensify appropriate medical therapy and |
| >10/24 hrs | monitor as clinically indicated. Stop serabelisib |
| Life-threatening | dosing until recovery to Grade ≤1, then resume |
| consequences | serabelisib at the lowest dose (300 mg) level. If |
| | 300 mg, resume 300 mg dose level, but if there is |
| | subject is already at recurrent vomiting or nausea of |
| | any Grade, stop serabelisib permanently. |

TABLE 11
Dose Modification and Management for Diarrhea

| Grade | Recommendation |
|---|---|
| Grade 1 | No serabelisib dose adjustment required. Provide medical |
| <4 stools/day increase over | therapy as needed and monitor as clinically indicated. |
| baseline or mild increase in | |
| ostomy output | |
| Grade 2 | Initiate or intensify appropriate medical therapy and monitor |
| Increase of 4-6 stools/day or | as clinically indicated. Stop serabelisib dosing until recovery |
| moderate increase in ostomy | to Grade ≤1, then resume serabelisib at same dose level. |
| output over baseline; limiting | After resumption, if recurs at Grade >1, stop serabelisib and |
| instrumental ADL | resume at lower dose level. If subject is already at 300 mg |
| | dose level, monitor for no more than 14 days and, if no |
| | resolution despite medical management, discontinue |
| | serabelisib. |
| Grade 3 | Initiate or intensify appropriate medical therapy and monitor |
| Increase of ≥7 stools/day or | as clinically indicated. Stop serabelisib dosing until recovery |
| severe increase in ostomy | to Grade ≤1, then resume serabelisib at the next lower dose |
| output over baseline; | level. After resumption, if recurs at Grade >1, stop |
| hospitalization indicated; | serabelisib and resume at next lower dose level. If subject is |
| limiting self-care ADL | already at 300 mg, resume 300mg dose level, but if there is |
| | recurrent diarrhea of ≥Grade 2, stop serabelisib |
| | permanently. |
| Grade 4 | Initiate or intensify appropriate medical therapy and monitor |
| Life-threatening | as clinically indicated. Stop serabelisib dosing until recovery |
| consequences; urgent | to Grade ≤1, then resume serabelisib at the lowest dose |
| intervention indicated | (300 mg) level. If subject is already at 300 mg, resume |
| | 300 mg dose level, but if there is recurrent diarrhea of any |
| | Grade, stop serabelisib permanently. |

Liver Enzyme Increase: ALT and AST (aminotransferase or AT) increases have been reported in subjects receiving serabelisib in previous clinical trials. The dose modifications in TABLE 12 assume that there are no alternative causes of increased transaminases such as concomitant meds, viral hepatitis, or progressive disease. If bilirubin increases or INR increases accompany transaminasemia, drug-induced liver injury (DILI; based on Hy's law) is suspected. Briefly, Hy's Law cases have the following three components: 1. The drug causes hepatocellular injury, generally shown by a higher incidence of 3-fold or greater elevations above the ULN of ALT or AST than the (non-hepatotoxic) control drug or placebo 2. Among trial subjects showing such AT elevations, often with ATs much greater than 3×ULN, one or more also show elevation of serum total bilirubin (TBL) to >2×ULN, without initial findings of cholestasis (elevated serum alkaline phosphatase) 3. No other reason can be found to explain the combination of increased AT and TBL, such as viral hepatitis A, B, or C; preexisting or acute liver disease; or another drug capable of causing the observed injury. Although no cases of DILI have been reported in prior serabelisib clinical trials, the dose modifications for AT increases assume that there are no concomitant increases in TBL or coagulation abnormalities and the criteria to suspect DILI are not met. TABLE 12 shows dose modification and management for ALT or AST transaminasemia.

TABLE 12
Dose Modification and Management for ALT or AST Transaminasemia

| Grade | Recommendation |
|---|---|
| Grade 1 | No serabelisib dose adjustment required. Provide medical |
| >ULN-3.0 × ULN if normal | therapy as needed and monitor as clinically indicated. |
| baseline; 1.5-3.0 × baseline if | |
| baseline was abnormal | |
| Grade 2 | No serabelisib dose adjustment required. Provide medical |
| >3.0-5.0 × ULN if normal | therapy as needed and monitor as clinically indicated. |
| baseline; >3.0-5.0 × baseline if | |
| baseline was abnormal | |
| Grade 3 | Initiate or intensify appropriate medical therapy and |
| >5.0-20.0 × ULN if normal | monitor as clinically indicated. Stop serabelisib dosing |
| baseline; >5.0-20.0 × baseline | until recovery to Grade ≤1, then resume serabelisib at |
| if baseline was abnormal | next lower dose level. After resumption, if recurs at |
| | >Grade 1, stop serabelisib until ≤Grade 1, and resume at |
| | next lower dose level. If subject is already at 300 mg dose |
| | level, monitor for no more than 14 days and, if ≥Grade 2 |
| | with no resolution, discontinue serabelisib. |
| Grade 4 | Initiate or intensify appropriate medical therapy and |
| >20.0 × ULN if normal | monitor as clinically indicated. Stop serabelisib dosing |
| baseline; >20.0 × baseline if | until recovery to Grade ≤1, then resume serabelisib at the |
| baseline was abnormal | lowest dose (300 mg) level. If subject is already at |
| | 300 mg, stop serabelisib permanently. |

Although hyperglycemia has been reported in subjects receiving serabelisib in previous clinical trials, in this study hyperglycemia is not expected because all subjects receive Study ISD. Metabolic monitoring is initiated on day 1. Pre-prepared meals are provided to study subjects with the counseling and monitoring of the study dietitian. However, if subjects do develop hyperglycemia, then subjects are monitored with blood glucose or fasting plasma glucose measurements to determine whether additional treatment with metformin is indicated. Metformin is not initiated if eGFR is <45 mL/min/1.73 m², and is discontinued or reduced in dose by 50% if a subject's eGFR decreases to this level. Metformin is used 500 mg once daily (before dinner) then titrate based on response and GI side effects up to a maximum of 1000 mg twice a day (BID).

If subjects are unable to reach glycemic goals despite treatment with metformin, another antihyperglycemia medication is added (TABLE 13). Antihyperglycemia agents that do not affect the PI3K pathway, such as acarbose and sodium-glucose cotransporter 2 inhibitors are considered. TABLE 14 lists secondary non-insulin agents for antihyperglycemia; TABLE 15 lists tertiary non-insulin agents for antihyperglycemia; TABLE 16 lists insulin for antihyperglycemia treatment. SGLT2 inhibitor treatment reduces renal glucose threshold, leading to glucosuria, reduced plasma glucose, and lower insulin levels. SGLT2 inhibitor treatment thus ameliorates the insulin feedback mechanism associated with PI3K inhibition. The combination of metformin and SGLT2 is safe and is widely used in treating subjects with hyperglycemia, but this combination has not been formally tested in subjects who were being treated with PI3K inhibitors. The development of euglycemic ketoacidosis (increased anion gap metabolic acidosis, ketonemia [>3 mM], or ketonuria [moderate to large on urinalysis], but with normal or modestly elevated blood glucose [<250 mg/dL or 13.9 mmol/L]) in a subject with breast cancer taking a PI3Ki with an SGLT2i. Subjects are counseled on the symptoms of ketoacidosis, including malaise, fatigue, nausea, and vomiting and may be advised to measure ketones using a blood ketone meter (β-hydroxybutyrate goal <0.6-3.0 mmol/L) or urine (acetoacetate) testing. Notably, nausea and vomiting are common alpelisib-associated AEs that may be difficult to distinguish from symptoms of ketoacidosis. Onset of ketoacidosis prompts immediate referral to an urgent care center or emergency room.

TABLE 13
Serabelisib Dose Reduction Recommendations for Hyperglycemia

| Fasting Plasma Glucose | |
|---|---|
| (FPG)/Blood Glucose | |
| Values | Recommendation |
| Grade 1 | No dose adjustment required. |
| FPG >ULN-160 mg/dL | Initiate metformin 500 mg AC QD (titrate up by 500 mg QD |
| or >ULN-8.9 mmol/L | weekly as needed and tolerated to max 2000 mg per day) |
| Grade 2 | No dose adjustment required. |
| FPG >160-250 mg/dL | Initiate/maximize metformin and add secondary non-insulin |
| or >8.9-13.9 mmol/L | agent |
| | If persistent Grade 2 hyperglycemia, add another secondary |
| | non-insulin agent or a tertiary non-insulin agent. |
| | If FPG does not decrease to ≤160 mg/dL or 8.9 mmol/L |
| | within 21 days under appropriate antidiabetic treatment, reduce |
| | dose by 1 dose level and follow FPG value specific |
| | recommendations. |
| Grade 3 | Interrupt serabelisib. |
| >250-500 mg/dL | Administer intravenous hydration and consider appropriate |
| or >13.9-27.8 mmol/L | treatment (e.g., intervention for |
| | electrolyte/ketoacidosis/hyperosmolar disturbances which may |
| | include short-term insulin). |
| | Initiate/maximize metformin, add secondary non-insulin agent |
| | plus additional secondary non-insulin agent or tertiary non- |
| | insulin agent. |
| | If FPG decreases to ≤160 mg/dL or 8.9 mmol/L within 3 to 5 |
| | days under appropriate anti-diabetic treatment, resume |
| | serabelisib without dose adjustment for first grade 3 |
| | occurrence. If second or third occurrence, resume at 1 or 2 |
| | lower dose levels, respectively. |
| | If FPG improves to grade 2 (160-250 mg/dL or 8.9-13.9 |
| | mmol/L) after addition of secondary non-insulin agent, follow |
| | dose modification and management for grade 2 hyperglycemia |
| | (see above). |
| | If FPG does not decrease to ≤160 mg/dL or 8.9 mmol/L |
| | within 3 to 5 days, initiate/maximize non-insulin antidiabetic |
| | treatment or, if needed, add insulin. |
| | If FPG does not decrease to ≤160 mg/dL or 8.9 mmol/L |
| | within 21 days following antidiabetic treatment, permanently |
| | discontinue serabelisib administration. |
| Grade 4 | Interrupt serabelisib. |
| >500 mg/dL | Administer intravenous hydration and consider appropriate |
| or ≥27.8 mmol/L | treatment (e.g., intervention for |
| | electrolyte/ketoacidosis/hyperosmolar disturbances which may |
| | include short-term insulin), re-check FPG within 24 hours and |
| | as clinically indicated. |
| | Initiate/maximize metformin, add secondary non-insulin agent |
| | plus additional secondary non-insulin agent or tertiary non- |
| | insulin agent. Add insulin. |
| | If FPG decreases to ≤500 mg/dL or 27.8 mmol/L, follow FPG |
| | value specific recommendations for Grade 3. |
| | If FPG is remains at >500 mg/dL or 27.8 mmol/L after IV |
| | hydration and acute antidiabetic treatment, permanently |
| | discontinue serabelisib administration. |

TABLE 14
Secondary Non-Insulin Agents for Antihyperglycemia Treatment

| | Risk for | | Other |
|---|---|---|---|
| | weight | Drug and recommended | Considerations |
| Class | loss | dosage | for Treatment |
| Sodium-Glucose | Yes | Ertugliflozin 5-15 mg | Monitor for |
| Cotransporter | | Dapagliflozin 5-10 mg | euglycemic |
| Inhibitor | | Canagliflozin 100-300 mg | ketoacidosis |
| | | Empagliflozin 10-25 mg | especially when |
| | | | subject is |
| | | | nauseous, |
| | | | vomiting, or |
| | | | dehydrated. |
| | | | Hold SGLT2i if |
| | | | with poor |
| | | | hydration and/or |
| | | | illness |
| | | | (infection, etc.) |
| | | | or 5 days prior |
| | | | to any surgery. |
| | | | Associated with |
| | | | Genitourinary |
| | | | infections |
| | | | Polyuria with |
| | | | risk of volume |
| | | | depletion and |
| | | | hypotension |
| | | | Glycosuria with |
| | | | risk of |
| | | | genitourinary |
| | | | infections |
| | | | including |
| | | | Fournier's |
| | | | gangrene in |
| | | | diabetics |
| DPP-4 inhibitors | No | Alogliptin 25 mg | Typically well |
| | | Saxagliptin 5 mg | tolerated |
| | | Linagliptin 5 mg | Theoretical risk |
| | | Sitagliptin 100 mg | of acute |
| | | | pancreatitis |
| | | | Joint pain |
| Thiazolidinediones | No | Pioglitazone 45 mg | Exercise |
| | | Rosiglitazone 4 mg | extreme caution |
| | | | when using in |
| | | | subjects with or |
| | | | at risk for |
| | | | congestive heart |
| | | | failure and |
| | | | subjects at risk |
| | | | falls/fractures |
| GLP-1 receptor | Yes | Exenatide (extended | GI AEs are |
| agonists | | release) 2 mg powder for | common |
| | | suspension or pen | (nausea, |
| | | Exenatide 10 μg pen | vomiting, |
| | | Dulaglutide 1.5/0.5 mL pen | diarrhea) |
| | | Semaglutide 1 mg pen | Injection site |
| | | Liraglutide 18 mg/3 mL pen | reactions |
| | | | Risk of |
| | | | pancreatitis |
| | | | Risk of thyroid |
| | | | C-cell tumors |
| α-Glucosidase | No | Acarbose 100 mg | GI AEs are |
| inhibitors | | | common |

TABLE 15
Tertiary Non-Insulin Agents for Antihyperglycemia Treatment

| | Risk for | Drug and | Other |
|---|---|---|---|
| | weight | recommended | Considerations for |
| Class | loss | dosage | Treatment |
| Meglitinides | No | Nateglinide | Potential AEs include: |
| | | 60-120 mg AC | Hypoglycemia |
| | | Repaglinide | Weight Gain |
| | | 0.5-4 mg AC | Diarrhea |
| | | | Nausea |
| Sulfonylureas | No | Glimepiride | Potential AEs include: |
| | | 1-8 mg PO OD | Hypoglycemia |
| | | Glipizide 5-40 | Weight Gain |
| | | mg PO (OD or | Diarrhea |
| | | split dose BID) | Nausea |
| | | Glipizide XR | |
| | | 5-20 mg PO | |
| | | Glyburide* | |
| | | 2.5-20 mg OD | |
| | | Glyburide | |
| | | micronized* | |
| | | 3-12 mg OD | |

TABLE 16
Insulin for Antihyperglycemia Treatment

| | Risk for | | | | Other |
|---|---|---|---|---|---|
| | weight | | Recommended | | Considerations |
| Class | loss | Drug | starting dose | | for Treatment |
| Basal | No | Insulin | 0.2 units per kg SC | | Potential AEs |
| insulin | | glargine | OD | | include: |
| | | Insulin | Increase for goal | | Hypoglycemia |
| | | detemir | fasting FSBG <160 | | Weight Gain |
| | | | mg/dL | | |
| Rapid | No | Insulin aspart | Start sliding scale AC | | Potential AEs |
| acting | | Insulin lispro | FPG | Units | include: |
| insulin | | Insulin | 70-99 | 0 | Hypoglycemia |
| | | glulisine | 100-149 | 0 | Weight Gain |
| | | | 150-199 | 2 | |
| | | | 200-249 | 3 | |
| | | | 250-299 | 4 | |
| | | | 300-349 | 5 | |
| | | | 350-399 | 6 | |
| | | | over 400 | 7 | |

Constipation is managed as per routine oncologic care. A dietitian is consulted is recommended and use of products with no added sugar like psyllium. The Study RD monitors for diabetic ketoacidosis using POC capillary BHB that is reported on the App. TABLE 17 provides a monitoring plan and recommendations for diabetic ketoacidosis.

TABLE 17
Monitoring Plan and Recommendations for Diabetic Ketoacidosis

| BHB (mM) | Recommendation |
|---|---|
| >3.0 | RD informs site of BHB level and inform site of recommendations made |
| | to the subject |
| | RD asks about signs or symptoms of dehydration or acute illness. |
| | RD reviews daily carbohydrate content with the subject |
| | RD encourages additional 3 liters of oral hydration daily and continues to |
| | monitor daily POC BHB |
| >3.5 | RD informs site of BHB level and inform site of recommendations made |
| | to the subject |
| | RD asks about signs or assesses for signs and symptoms of dehydration |
| | or acute illness. |
| | RD alerts study dietitian and reviews daily carbohydrate content with the |
| | subject |
| | If concerning signs and symptoms present, study team obtain basic |
| | metabolic panel to assess for presence of gap metabolic acidosis |
| | If HCO₃_{^{−}} <18 mEq/L with anion gap >16, hold serabelisib, and refer to |
| | acute care medical facility |
| | If HCO₃_{^{−}} >18 mEq/L, encourage additional 3 liters of oral hydration daily, |
| | add 10 g of carbohydrate daily, and continue daily POC BHB monitoring |

Urolithiasis is always be considered in the differential diagnosis of abdominal pain. The classic presentation of renal colic is excruciating unilateral flank or lower abdominal pain of sudden onset that is not related to any precipitating event and is not relieved by postural changes or nonnarcotic medications. With the exception of nausea and vomiting secondary to stimulation of the celiac plexus, gastrointestinal symptoms are usually absent.

Urinalysis is performed in all subjects with suspected calculi. Aside from the typical microhematuria, important findings to note are the urine pH and the presence of crystals, which help to identify the stone composition. Several imaging modalities are available to confirm the diagnosis. The first step is to identify subjects who require emergency urologic consultation. For example, sepsis in conjunction with an obstructing stone represents a true emergency. Other emergency conditions are anuria and acute renal failure secondary to bilateral obstruction, or unilateral obstruction in a subject with a solitary functioning kidney.

Analgesia and intravenous hydration are the mainstays of non-emergent therapy. Referral to a urologist is indicated for subjects with a stone greater than 5 mm in size. Referral is also indicated for subjects with a ureteral stone that has not passed after two to four weeks of observation.

### Example 3: Use of Application to Assist and Assess Subjects

Subjects receive detailed instructions from the study team on how to take medications at home and also get trained to use a unique smart-phone or tablet-enabled application called “the App” which is used by the subject to manually enter multiple data including: daily drug intake; body weight; capillary ketone body β-hydroxybutyrate (BHB); capillary glucose; and surveys on quantity eaten, quality of, and satisfaction with meals.

The App is also used by subjects to communicate with the study team regarding informing on food quality or delivery status and connecting with the study team regarding questions about meals and seeking information for dietary support. Subjects are instructed to return all used and unused Study Drug containers at day 1 of each cycle when they receive their next dispensation of drug. Subject compliance with the dosing schedule is assessed by reconciliation of the used and unused Study Drug at each clinic visit and review of the App data. Compliance is be monitored and documented by site personnel on the appropriate form. The site personnel determines the subject's adherence to the dosing schedule by reviewing the App data and questioning the subject about any discrepancies, recording the number of tablets and strengths returned, the date returned, and determining intervention compliance before dispensing new medication to the study subject.

Subjects use the App to record actual daily food intake and surveys on the quality and satisfaction with meals. The study dietitian reviews meal consumption to guide caloric intake and subject satisfaction. The App is also used by subjects to record their body weight, capillary ketone body β-hydroxybutyrate (BHB), and serum glucose. In addition, the App allows communication with the dietitian to ask questions about the meals, and enables communication with the operations team to report on food quality or delivery concerns. Lastly, the App provides delivery tracking and updates for subjects regarding status of the shipments.

The importance of Study Drug compliance is emphasized to the subject. Subjects receive detailed instructions on how to take medications at home and utilize the App to record daily drug intake. Subjects are instructed to return all used and unused Study Drug containers at each study visit. Subject compliance with the dosing schedule is assessed by reconciliation of the used and unused Study Drug at each clinic visit and review of the App data. Compliance is monitored and documented by site personnel on the appropriate form. The site personnel determines the subject's adherence to the dosing schedule by reviewing the App data and questioning the subject about any discrepancies, recording the number of tablets and strengths returned, the date returned, and determining Study Drug compliance before dispensing new medication to the study subject.

### Example 4: Prior and Concomitant Treatments

All prescription and over-the-counter (OTC) medications taken by a subject within 30 days before the first administration of Study Drug are recorded on the designated eCRF. The following medications/therapies are prohibited:
Other investigational agents
Other anticancer therapies (drugs, biologics, radiation, or surgery)
Strong CYP3A4 inducers/inhibitors
Histamine-H2 receptor antagonists within 24 hours of Study Drug administration or PPIs
Antacids within 24 hours of Study Drug administration

Prophylactic antiemetic therapy is not used with the Visit 3 (Cycle 1, Study Day 1) dose. If a subject experiences any vomiting or nausea, antiemetic medications may be administered in keeping with standard clinical practice. Hematopoietic growth factors, transfusions of blood and blood products are not used in particular during Cycle 1 unless necessary, but can be administered starting from Cycle 2 and onward, if needed.

Subjects who develop hyperglycemia while on study are monitored closely to determine whether treatment with metformin is indicated, after discussion with the Sponsor's Medical Monitor. Other medications considered necessary for the subject's safety and well-being are given at the discretion of the Investigator. Any concomitant medications added or discontinued during the study are recorded on the eCRF.

### Example 5: Use of Serabelisib in Combination with an Insulin Suppressing Diet and with or without Nab-Paclitaxel in Subjects with Cancer

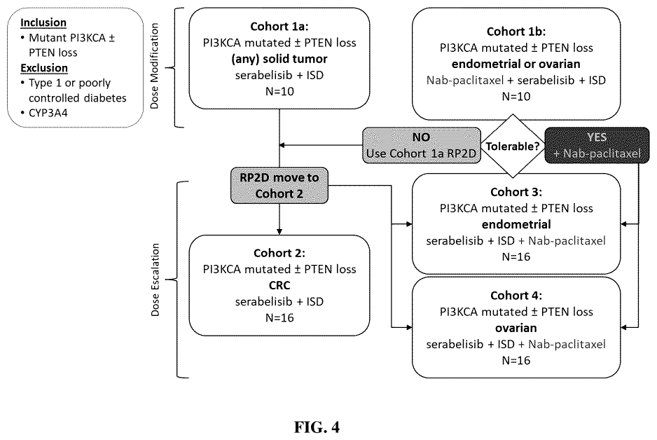

The primary objective of the study is to evaluate the safety, feasibility, and pharmacokinetic impact of intrasubject modulated dosing for serabelisib when co-administered with a study ISD in a pilot cohort of subjects with solid tumors with PIK3CA mutations with or without PTEN loss. An expansion cohort will include subjects with solid tumors that have failed standard-of-care (SOC) therapy and possess PIK3CA-activating mutations with or without PTEN loss: colorectal cancer (CRC) (Cohort 2), endometrial cancer (EC) (Cohort 3), and ovarian clear cell or ovarian endometrioid carcinoma (Cohort 4). For the expansion cohort, nab-paclitaxel will also be administered with serabelisib when co-administered with a study ISD. TABLE 18 shows a summary of the protocol for this study. FIG. 4 depicts a representation of the study cohorts of the clinical trial using serabelisib in combination with an insulin suppressing diet and nab-paclitaxel in subjects with cancer.

TABLE 18
Protocol Summary

| | | | | Serabelisib | Nab-paclitaxel | |
|---|---|---|---|---|---|---|
| | | | | oral (PO) in | intravenously | |
| | | Approximate | | divided doses on | 3-weeks-on/ | |
| | | number of | Cancer/ | 3 consecutive | 1-week-off | |
| | | evaluable | Mutational | days per week | schedule | Study |
| | Cohort | subjects | Status | (3CDW) | (3Q4W) | ISD |
| | Dose Modification: 20 subjects | | | | | |
| Cohort 1 Dose | 1a | 10 | PIK3CA- | 600 mg starting | No | Yes |
| Modification | | | mutated ± | dose (administered | | |
| | | | PTEN loss | PO as 300 mg after | | |
| | | | solid tumors | breakfast and 300 | | |
| | | | | mg after lunch), | | |
| | | | | adjusted to 900 mg | | |
| | | | | (administered PO | | |
| | | | | as 300 mg after | | |
| | | | | breakfast, lunch, | | |
| | | | | and dinner), remain | | |
| | | | | at 600 mg, or de- | | |
| | | | | escalate to 300 mg | | |
| | | | | (administered PO | | |
| | | | | as 300 mg after | | |
| | | | | breakfast) based | | |
| | | | | on safety | | |
| | 1b | 10 | PIK3CA- | 600 mg starting | 80 mg/m² | Yes |
| | | | mutated ± | dose (administered | (administered | |
| | | | PTEN loss | PO as 300 mg after | according to | |
| | | | endometrial | breakfast and 300 | prescribing | |
| | | | cancer, | mg after lunch), | information; | |
| | | | ovarian | adjusted to 900 mg | intravenously | |
| | | | clear cell, | (administered PO | over 30 minutes), | |
| | | | or ovarian | as 300 mg after | dose modifications | |
| | | | endometrioid | breakfast, lunch, | based on AEs | |
| | | | carcinoma | and dinner), remain | and laboratory | |
| | | | | at 600 mg, or de- | evaluations | |
| | | | | escalate to 300 mg | | |
| | | | | (administered PO | | |
| | | | | as 300 mg after | | |
| | | | | breakfast) based | | |
| | | | | on safety | | |
| | Cohort Expansion: 48 subjects | | | | | |
| Cohorts 2, | 2 | 16 | PIK3CA- | Recommended | No | Yes |
| 3, and 4 | | | mutated ± | Phase 2 dose | | |
| Cohort | | | PTEN loss | (RP2D) | | |
| Expansion | | | colorectal | | | |
| | | | cancer | | | |
| | 3 | 16 | PIK3CA- | RP2D | May be added | Yes |
| | | | mutated ± | | depending on | |
| | | | PTEN loss | | results from | |
| | | | endometrial | | Cohort 1b | |
| | | | cancer | | | |
| | 4 | 16 | PIK3CA- | RP2D | May be added | Yes |
| | | | mutated ± | | depending on | |
| | | | PTEN loss | | results from | |
| | | | ovarian clear | | Cohort 1b | |
| | | | cell or ovarian | | | |
| | | | endometrioid | | | |
| | | | carcinoma | | | |

The study takes place in up to 15 study sites in the United States. The total planned enrollment is approximately 68 evaluable subjects (dose modification=20; cohort expansion=48).

Cohort 1a (Dose Modification): The primary objective of the study is to evaluate the safety, compliance, and pharmacokinetic (PK) impact of intrasubject modulated dosing for serabelisib in combination with a Study ISD in a pilot cohort. Endpoints include: Incidence of related adverse events (AEs); Compliance of study intervention; and Standard PK parameters (including but not limited to Cₘₐₓ, Tₘₐₓ, and AUC). The secondary objective of the study is to assess the antitumor efficacy of serabelisib in combination with a Study ISD. Endpoints include: ORR, defined as the proportion of subjects who have best overall response of either CR or PR, as determined by each site; Progression free survival (PFS), defined as the time from date of first dose of Study Drug (Visit 1) to the date of first evidence of disease progression or death from any cause (whichever occurs first); Overall survival (OS) and landmark survival; Duration of response (DoR); and Disease control rate (DCR; CR+PR+stable disease [SD]).

Cohort 1b (Dose Modification): The primary objective of the study is to evaluate the safety, compliance, and pharmacokinetic (PK) impact of intrasubject modulated dosing for serabelisib in combination with a Study ISD and once weekly nab-paclitaxel in a pilot cohort. Endpoints include: Incidence of related adverse events (AEs); Compliance of study intervention; and Standard PK parameters (including but not limited to Cₘₐₓ, Tₘₐₓ, and AUC). The secondary objective of the study is to assess the antitumor efficacy of serabelisib in combination with a Study ISD and once weekly nab-paclitaxel in a pilot cohort. Endpoints include: ORR, defined as the proportion of subjects who have best overall response of either CR or PR, as determined by each site; Progression free survival (PFS), defined as the time from date of first dose of Study Drug (Visit 1) to the date of first evidence of disease progression or death from any cause (whichever occurs first); Overall survival (OS) and landmark survival; Duration of response (DoR); and Disease control rate (DCR; CR+PR+stable disease [SD]).

Cohorts 2, 3, and 4 (Cohort Expansion): The primary objective of the study is to assess the antitumor efficacy of serabelisib in combination with a Study ISD. The secondary objective of the study is to confirm the safety and compliance of serabelisib in combination with a Study ISD, and to provide additional assessments of antitumor efficacy of serabelisib in combination with a Study ISD. Endpoints include: Incidence of related AEs; Compliance of study intervention; PFS, defined as the time from date of first dose of Study Drug (Visit 1) to the date of first evidence of disease progression or death from any cause (whichever occurs first); OS and landmark survival; DoR; and DCR (CR+PR+SD). Another secondary objective of the study is to assess the population PK and intra-tumoral concentration of serabelisib in combination with a Study ISD. Endpoints include: Standard PK parameters of serabelisib (including but not limited to Cₘₐₓ, Tₘₐₓ, and AUC) in plasma and serabelisib concentration in tumor tissue.

All Cohorts—1a, 1b, 2, 3, and 4: An exploratory objective of these studies is to assess the pharmacodynamics (PD) of serabelisib in combination with a Study ISD. Endpoints include: Characterization of changes in fasting blood glucose, insulin, and biomarkers of insulin-PI3K signaling in blood, buccal cells, and tumor biopsies; changes in tumor marker levels in blood. Another exploratory objective of these studies is to assess the ability of serabelisib in combination with a Study ISD to upregulate the immune response. Endpoints include: Assessment of immune markers in PBMCs at baseline, during the ISD run in, and after the addition of serabelisib and paclitaxel (if added). A third exploratory objective of these studies is to assess genetic predictors of toxicity and efficacy. Toxicity and efficacy data will direct an analysis into whether the genetic status of the patient predicts a positive or negative therapeutic response (efficacy) or susceptibility to AE.

Investigational product, dose, and mode of administration: The Study Drug, serabelisib (6-(2-Aminobenzo[d]oxazol-5-yl) imidazo[1,2-a]pyridine-3-yl morpholino methanone) is a class I PI3Kα inhibitor. Serabelisib will be supplied as immediate-release tablets for PO administration in 300 mg dose strengths. The Study ISD will be supplied as pre-packaged meals delivered to the subject's home on a weekly basis. The Study Drug will be given in 300 mg tablets to be taken PO on 3 consecutive days per week (3CDW) beginning on either Monday, Tuesday, or Wednesday (MTW) followed by 4 consecutive days off Study Drug. Subjects should be instructed to take the Study Drug on the same 3 consecutive days of the week for all dosing. Subjects will consume the dose of serabelisib within 15 minutes of finishing the Study ISD meal. The Study Drug must be consumed continuously on the 3CDW schedule without a break. The Study ISD will be delivered weekly and supplied as 3 pre-prepared meals (i.e., breakfast, lunch, dinner) and optional snacks provided depending on caloric needs. The meals must be consumed orally at the subject's usual times for breakfast, lunch, and dinner with regular monitoring for efficacy (e.g., insulin suppression) using routine clinical biomarkers. The Study ISD must be consumed as directed each day without a break, whether or not the subject is taking serabelisib. Alcohol, while not specifically controlled or dictated, is not part of the strict Study ISD and should be discouraged.

Study Rationale: Targeted anticancer drugs have side effects that often result in a poor quality of life, noncompliance, dose decreases, or discontinuation, all of which can affect efficacy. This study will evaluate the feasibility of optimizing the safety and tolerability of serabelisib when combined with an ISD with a goal of reducing side effects and enhancing anticancer activity.

Serabelisib is a small molecule with potent and selective inhibitory activity against the PI3Kα isoform, and can be a promising anticancer monotherapy. Combining PI3K inhibition with an ISD can result in improved efficacy compared with PI3K inhibition alone. Specifically, the combination of serabelisib and the Study ISD will be evaluated to determine safety and efficacy in adult subjects with advanced, PIK3CA-mutated solid tumors with or without PTEN loss.

In addition, preclinical and clinical studies in breast and gynecological tumors suggest that inhibition of the PI3K/AKT/mTOR pathway may reduce resistance to taxanes and that inhibition of that pathway can re-sensitize tumors to the effects of paclitaxel. Thus, Cohort 1b will assess whether the addition of weekly nab-paclitaxel to serabelisib and Study ISD is tolerable and efficacious in patients with advanced ovarian or endometrial tumors. In addition to determination of optimal serabelisib dose when combined with the Study ISD (dose optimization in Cohort 1), the safety, tolerability, compliance, and efficacy of combining serabelisib with the Study ISD will be evaluated (expansion in Cohorts 2, 3, and 4).

Serabelisib's pharmacokinetic (PK), dose regimen, and toxicity profile were evaluated in prior studies in normal healthy volunteers and cancer subjects in 4 separate studies. In these studies, a total of 171 solid tumor cancer subjects and 54 healthy volunteers have received serabelisib under a variety of conditions, doses, and regimens.

Based on a study in 125 subjects with solid tumors (not selected by PIK3CA mutations or PTEN loss), the proposed single agent recommended Phase 2 dose (RP2D) was 900 mg serabelisib on each of three consecutive days each week (3CDW), continuously. Results from skin biopsy samples suggest that serabelisib exposures achieved with doses of >200 mg will be biologically active. On the 3CDW schedule, serabelisib was shown to be reasonably well tolerated with less toxicity compared with other schedules evaluated, and toxicity appeared to be greater with increased dose intensity. The most common related adverse events (AEs) across all doses and regimens with serabelisib included nausea (50%), vomiting (38%), hyperglycemia (37%), diarrhea (31%), decreased appetite (23%), and increased aspartate aminotransferase (AST) (20%). In the 3CDW cohorts, 7% of subjects had related serious adverse events (SAEs). When the dose was escalated from 900 mg to 1200 mg in the 3CDW regimen, 2 of 3 subjects had gastrointestinal (GI) dose-limiting toxicity (DLT). Based upon these AE findings, serabelisib will be administered orally (PO) on either a twice daily (BID) or three times daily (TID) regimen in order to reduce the initial Cₘₐₓ concentration that may be associated with either nausea or vomiting. Divided dose administration is also anticipated to reduce the rate of rise over the first two dosing intervals. The linear PK profile of serabelisib also suggests that near steady-state concentrations will be achieved on day 3 of the 3CDW cycle. No meaningful drug accumulation is expected between each dosing cycle. Dosing regimens of either 300 mg PO BID or TID are expected to maintain drug exposure within the target total drug concentration window of about 10-20 μM. The proposed meals in the Study ISD described herein are not expected to compromise drug bioavailability (BA).

Nab-paclitaxel: Preclinical studies suggest that activation of the PI3K/AKT/mTOR pathway by taxanes may play a role in the development of taxane resistance and that inhibition of that pathway can re-sensitize tumors to the effects of paclitaxel. A retrospective analysis in patients with breast and gynecological cancer appears to confirm that treatment with PI3K or mTOR inhibitors may help to increase efficacy of subsequent taxane treatment.

In a phase 1 study in 16 patients with advanced ovarian, endometrial, or breast cancer, the combination of serabelisib with sapanisertib, a TORC 1/2 inhibitor and paclitaxel showed that this regimen was overall safe and tolerable, with only 28 (8%) grade 3 or 4 adverse events. The most common events were leukopenia and non-febrile neutropenia. Two patients required dose reductions as a result of pneumonitis. The ORR was 46% in 13 evaluable patients, the clinical benefit rate was 69% and PFS was at 10 months at the time of data analysis. Remarkably, 2 patients achieved a CR.

Similarly, in a phase 1 study in 43 HER2-negative patients with advanced breast cancer of whom 84% had been treated with taxanes previously, the combination of the PI3Kα inhibitor alpelisib with nab-paclitaxel proved generally well tolerated, the most frequent adverse events being hyperglycemia (26% grade 3, no grade 4), neutropenia (23% grade 3, 7% grade 4), diarrhea (5% grade 3, no grade 4) and rash (7% grade 3, no grade 4). ORR was 59% (CR 7%), 21% of whom had response lasting >12 months; median PFS was 8.7 months. A total of 40% of patients had histological or circulating tumor DNA (ctDNA) based evidence for PIK3CA mutations; patients with tumor/ctDNA mutation demonstrated better PFS compared with those without mutation (11.9 vs. 7.5 months; HR, 0.44; P=0.027).

Unlike other tubulin-targeting drugs, paclitaxel prevents microtubule polymer disassembly, and thus, blocks the progression of mitosis triggering apoptosis or reversion to the GO-phase of the cell cycle. Given that the activity of paclitaxel is directly related to the cell cycle, more frequent weekly administration of this drug can improve efficacy in patients with advanced ovarian cancer.

Nab-paclitaxel is an albumin-bound, solvent-free formulation of paclitaxel that does not require steroid premedication, which can improve the therapeutic index as compared to paclitaxel. In addition, nab-paclitaxel has an advantageous PK profile achieving a 33% higher tumor uptake in preclinical models. Although nab-paclitaxel is not labeled for use in the setting of relapsed/recurrent ovarian or endometrioid tumors, National Comprehensive Cancer Network (NCCN) clinical practice guidelines include nab-paclitaxel as a SOC that may be administered to patients with recurrent or metastatic cancer.

Dose Modification Strategy Evaluation: Dietary fat content has beneficial effects on the PK and GI tolerability of serabelisib. Therefore, an intrasubject dose-modification scheme (Cohort 1a; n=10; Cohort 1b; n=10) will be employed in evaluable subjects with any solid tumor (Cohort 1a) or endometrial cancer (EC) or ovarian clear cell or ovarian endometroid carcinoma (Cohort 1b) with PIK3CA mutation with or without PTEN loss to determine the safety, feasibility, and PK impact of intrasubject modulated dosing for serabelisib in combination with the Study ISD (Cohort 1a) and, in addition, with weekly nab-paclitaxel (Cohort 1b) to derive the optimal dosing regimen for the cohort expansion. Subjects will consume the Study ISD for at least 7 days (run-in period) before taking their first dose of serabelisib to allow for adjustment to the dietary change and assess the metabolic effects, tolerability, and adherence of the Study ISD prior to the first administration of serabelisib and remaining study procedures.

An intrasubject dose-modification scheme will be employed in 10 evaluable subjects in Cohort 1 to determine the optimal dose modification strategy for the combination of serabelisib and the Study ISD in the expansion cohorts (Cohorts 2, 3, and 4). All cohorts will consume the Study ISD for at least 7 days before the subject takes their first dose of serabelisib (run-in period), to allow for adjustment to the dietary change and assess the metabolic effects, tolerability, and adherence of the Study ISD prior to the first administration of serabelisib and remaining study procedures.

In all cohorts, all serabelisib doses will be divided, taken within 15 minutes after a meal, and all will be taken on a 3CDW regimen. Therefore, the 900 mg dose will be taken as 300 mg after breakfast, lunch, and dinner; 600 mg will be taken as 300 mg after breakfast and lunch; and the 300 mg dose will be taken as 300 mg after breakfast.

To be conservative, the starting dose of serabelisib in Cohort 1 will be 600 mg 3CDW (beginning on either Monday, Tuesday, or Wednesday), which is less than the previously recommended single agent Phase 2 dose (RP2D) of 900 mg once daily (QD) MTW/week. After two weeks of dosing, subjects may escalate to 900 mg (300 mg after breakfast, lunch, and dinner), remain at 600 mg, or de-escalate to 300 mg (300 mg taken after breakfast) 3CDW.

A dose modification decision to remain at 600 mg, increase to 900 mg, or decrease to 300 mg for a subsequent two weeks will be made based on AEs and laboratory evaluations. Subjects will not be allowed to receive a dose higher than 900 mg and if a subject cannot tolerate 300 mg, for whatever reason, they will be discontinued from the study. After at least 10 evaluable subjects have finished ≥56 days (i.e., two cycles) on serabelisib, the AEs, laboratory evaluations, biomarkers, PK, and efficacy parameters will be evaluated to determine the starting dose (i.e., RP2D) for Cohorts 2, 3, and 4.

Nab-paclitaxel will be administered intravenously weekly according to the prescribing information. The starting dose level of nab-paclitaxel in this study will be 80 mg/m² administered intravenously over 30 minutes (±5 minutes) on Days 1, 8, and 15 of each 28-day cycle (3-weeks-on/1-week-off schedule). Dose modifications should be performed based on AEs and laboratory evaluations.

Methodology: This is a multicenter, open-label study of serabelisib in combination with a Study ISD in subjects with advanced solid tumors with PIK3CA mutations with or without PTEN loss. In a subset of subjects (Cohort 1b, possibly Cohorts 3, 4), weekly nab-paclitaxel will be added to serabelisib and Study ISD. The total planned enrollment is approximately 68 evaluable subjects. Enrollment is defined as a subject who has been consented, screened, verified for eligibility, and initiated the Study ISD. Individual subjects will not be included in more than one cohort.

For all cohorts, the study will consist of three periods: screening and run-in (Visits-1 and 0, respectively), Study Drug administration (Visit 1 and onward), and end of intervention. During the Study Drug administration period, serabelisib must be consumed on a 3CDW schedule in 28-day (4-week ±3 d) cycles without a break between cycles.

Treatment with the Study ISD and serabelisib (and weekly nab-paclitaxel in a subset of subjects) can continue with additional cycles beyond Cycle 4, and will continue until subjects develop disease progression, have unacceptable toxicity, or for other protocol-specified reasons.

At Visit −1 (screening), subjects will provide informed consent and will undergo screening. The results obtained at screening will determine a subject's eligibility. At Visit 0 (run-in), blood will be collected to establish baseline values.

An intrasubject dose-modification scheme will be employed in Cohorts 1a (n=10) and 1b (n=10) to determine the appropriate serabelisib dose to combine with the Study ISD (Cohort 1a) and, in addition, with weekly nab-paclitaxel (Cohort 1b). The dosing regimen determined with Cohort 1a will be implemented with Cohort 2, and, in case the addition of weekly nab-paclitaxel in Cohort 1b proves intolerable and/or lacking additional clinical benefits, also with Cohorts 3 and 4 (n=16 per cohort; total of 48). If safety and efficacy results in Cohort 1b support the clinical utility of adding weekly nab-paclitaxel to serabelisib with study ISD, this regimen will be further studied in Cohorts 3 and 4 (n=16 per cohort; total of 32) while Cohort 2 (n=16) will continue to assess the utility of serabelisib with Study ISD alone based on the results from Cohort 1a.

While this study will enroll subjects with different tumor types and mutational status. No more than 8 subjects with PTEN loss will be allowed per cohort for Cohorts 2, 3, and 4.

Safety will be evaluated by AE, vital signs, clinical laboratory tests, electrocardiograms (ECG), and physical examination findings. Efficacy will be determined from tumor assessments (ORR, PFS, DCR, DOR, and OS). Pharmacokinetic parameters will be derived from timed blood sampling. Pharmacodynamic assessment will be completed using blood metabolites and hormones (glucose, insulin) and markers of tissue (tumor biopsy, buccal cells) level insulin signaling as well as changes in peripheral blood tumor biomarker levels.

Eligibility: Cohort 1a (Dose Modification): Adult subjects with any advanced solid tumor with PIK3CA mutations with or without PTEN loss. Cohort 1b (Dose Modification, addition of weekly nab-paclitaxel): Adult subjects with PIK3CA-mutations with or without PTEN loss, and advanced endometrial cancer, or advanced ovarian clear cell or ovarian endometroid carcinoma. Cohorts 2, 3, and 4 (Cohort Expansion): Adult subjects with PIK3CA-mutations with or without PTEN loss, and advanced colorectal cancer, advanced endometrial cancer, or advanced ovarian clear cell or ovarian endometrioid carcinoma.

Inclusion Criteria: Subjects are included only if they meet all of the following criteria:
1. Able to provide written informed consent.
2. Age ≥18 at Visit 1 (screening).
3. Histologically or cytologically confirmed recurrent solid tumors.
   a) Cohort 1a: any extracranial solid tumor
   b) Cohort 1b: either recurrent or persistent endometrial adenocarcinoma with the following histologic epithelial cell types: endometrioid adenocarcinoma, serous adenocarcinoma, undifferentiated carcinoma, clear cell adenocarcinoma, mixed epithelial carcinoma, adenocarcinoma not otherwise specified (N.O.S.), mucinous adenocarcinoma, squamous cell carcinoma, transitional cell carcinoma, and carcinosarcoma or; ovarian cancer primary tumor with ≥50% clear cell histomorphology or ovarian clear cell or ovarian endometrioid carcinoma
   c) Cohort 2: adenocarcinoma of the colon or rectum.
   d) Cohort 3: recurrent or persistent endometrial adenocarcinoma with the histologic epithelial cell types described for Cohort 1b
   e) Cohort 4: ovarian cancer primary tumor as described for Cohort 1b
4. Tumor must harbor an activating mutation in the PIK3CA gene with or without PTEN loss, either previously documented or determined during screening.
5. Fresh or archival tumor biopsy with sufficient material to be sent to the designated laboratory for PD analyses. If only slides are available, a minimum of 15 slides from a surgical specimen or a minimum of 20 slides from a biopsy is required. It is recommended to provide a tumor sample collected after the most recent progression or recurrence. For patients who consent to future research, an additional 5 slides from a surgical specimen or biopsy is required.
6. Cohort 1a—Dose Modification (subjects with any solid tumor): failed, were intolerant of, or ineligible for no more than 3 prior lines of therapy (LOT) for advanced/metastatic disease (American Joint Committee on Cancer [AJCC] stage III and IV) or refused standard-of-care (SOC) therapy.
7. For all cohorts, in the unlikely scenario that a subject refused all available SOC they may proceed with trial. These subjects would be regarded as having 0 prior LOT.
8. Cohorts 1b, 2, 3, and 4: failed, were intolerant of, ineligible for, or have refused SOC therapy for advanced/metastatic disease (AJCC stage III and IV) and:
   a) Cohort 2 (subjects with colorectal cancer): Have failed no more than 2 prior LOT for metastatic colorectal cancer.
   b) Cohort 1b, Cohort 3 (subjects with endometrial cancer): Subjects must have had no more than 3 prior chemotherapeutic regimens for management of endometrial carcinoma (neo-adjuvant and/or adjuvant chemotherapy will be counted as 1 prior LOT). Initial treatment may include chemotherapy, chemotherapy, and radiation therapy, and/or consolidation/maintenance therapy. Chemotherapy administered in conjunction with primary radiation as a radio-sensitizer WILL be counted as a systemic chemotherapy regimen. Bevacizumab given as part of initial therapy will count as part of initial therapy. Subjects may have up to two additional LOT for the treatment for advanced/metastatic/recurrent disease to include but not limited to anti-vascular endothelial growth factor (VEGF) targeted therapy [oo, cabozantinib, bevacizumab] and anti-programmed cell death protein 1 (PD-1) (e.g., dostarlimab, pembrolizumab, nivolumab) or anti-programmed death-ligand 1 (PD-L1) (e.g., avelumab, durvalumab) inhibitors, human epidermal growth factor receptor (HER) 2 targeted therapies (e.g., trastuzumab, trastuzumab emtansine, trastuzumab deruxtecan), mammalian target of rapamycin (mTor) inhibitors (e.g., temsirolimus, everolimus, ridaforolimus). Prior hormonal therapy will not count as a systemic regimen.
   c) Cohort 1b, Cohort 4 (subjects with clear cell or endometrioid ovarian cancer): Subjects must have had no more than 3 prior chemotherapeutic regimens for management of ovarian carcinoma (neo-adjuvant and/or adjuvant chemotherapy will be counted as 1 LOT). Initial treatment may include chemotherapy, chemotherapy plus maintenance poly(ADP-ribose) polymerase inhibitor (PARPi) maintenance therapy, and chemotherapy plus bevacizumab maintenance therapy. Subjects may have up to two additional LOT for the treatment for advanced/metastatic/recurrent disease to include but not be limited to chemotherapy, anti-VEGF targeted therapy (e.g., bevacizumab, pazopanib), PARPis (e.g., niraparib, olaparib, rucaparib), and anti-PD1 inhibitors (pembrolizumab, dostarlimab). Subjects must be platinum resistant/platinum refractory unless they had refused treatment. Breast cancer gene (BRCA) or homologous recombination deficiency (HRD) mutant subjects must have treatment with a PARPi. Prior hormonal therapy will not count as a systemic regimen.
9. Life expectancy of at least 3 months.
10. At least one measurable lesion (as defined by Response Evaluation Criteria in Solid Tumors [RECIST] 1.1). One of the lesions has to be in a location that allows for the on-study biopsy.
11. Eastern Cooperative Oncology Group performance status (ECOG PS) of 0 to 1.
12. Adequate organ function
   a) Absolute neutrophil count (ANC) ≥1.0×10⁹/L, platelet count ≥75 × 10⁹/L, and hemoglobin ≥8.5 gm/dL (may be transfused to reach this hemoglobin level unless due to blood loss).
   b) Liver transaminases (aspartate aminotransferase [AST] and alanine aminotransferase [ALT])≤2.5× upper limit of normal (ULN) (<5×ULN if liver metastases are present), and total bilirubin≤1.5×ULN (<3×ULN if subject has Gilbert Syndrome).
   c) International Normalized Ratio (INR)≤1.5×ULN unless subject is on anticoagulants that would affect the INR, then INR must be in the desired therapeutic range as judged by the Investigator.
   d) Albumin level ≥3.0 mg/dL or ≥the lower limit of normal.
   e) Renal: Serum creatinine ≤2×ULN (or if higher than the normal range, calculated creatinine clearance [CrCl] must be ≥60 mL/min/1.73 m² by body surface area-modified Cockcroft-Gault or other appropriate formula; actual body weight must be used for calculation of CrCl unless body mass index (BMI) is >30 kg/m², in which case, lean body weight must be used).
13. Ability to take oral medication, be willing to adhere to study procedures and Study Drug administration, and receive, consume, and comply with Study ISD.
14. For women of child-bearing potential (defined as a sexually mature woman who has not undergone hysterectomy or bilateral oophorectomy or has not been naturally postmenopausal for at least 24 consecutive months [i.e., has had menses at any time during the preceding 24 consecutive months]), a negative serum pregnancy test collected at screening (V-1) and negative urine pregnancy test collected at baseline (V) and use of physician-approved method of birth control from the time of the pregnancy test performed at screening to 90 days following the last administration of Study Drug.
15. Male subjects must be surgically sterile or must agree to use physician-approved contraception during the study and for 90 days following the last administration of Study Drug.

Exclusion Criteria: Subjects are not eligible if they meet any of the following criteria.
1. Diagnosis of primary brain tumor.
2. Has had serabelisib, alpelisib, or other PI3K inhibitor.
3. Leptomeningeal disease and symptomatic or untreated brain metastases. Subjects with treated (surgically excised or irradiated) and stable brain metastases are eligible, assuming adequate recovery from treatment to Grade 1 or less, the treatment was at least 28 days prior to first administration of Study Drug, and baseline brain computed tomography (CT) with contrast or magnetic resonance imaging (MRI) within 14 days of initiation of Study Drug, is negative for new or worsening brain metastases.
4. Diagnosis of, or requiring treatment for, another malignancy within the past 2 year (excluding a history of carcinoma in situ of the cervix, superficial non-melanoma skin cancer, or superficial bladder cancer that has been adequately treated, or stage 1 prostate cancer that does not require treatment or requires only treatment with luteinizing hormone-releasing hormone agonists or antagonists if initiated at least 90 days prior to the first dose of Study Drug).
5. Is less than 21 days from therapeutic radiation or chemotherapy prior to the first day of dosing with Study Drug and has not recovered to Grade≤1 (National Cancer Institute [NCI] Common Terminology Criteria for Adverse Events [CTCAE] v5.0) from all clinically significant toxicities related to prior therapies.
6. For subjects receiving nitrosoureas or mitomycin C, the subject is <6 weeks from last dose. For monoclonal antibody therapy, the subject is <one half-life or <4 weeks from the last dose.
7. Chronic, systemically administered glucocorticoids in doses equivalent to >5 mg prednisone daily. Topical, inhalational, ophthalmic, intraarticular, and intranasal glucocorticoids are permitted. Isolated or intermittent use of systemically administered glucocorticoids to treat complications of malignancy, use as a premedication, or as a one-time prep for an imaging procedure is permitted. If subject was on >5 mg prednisone/day equivalent, last dose must have been at least 7 days prior to the first dose of Study Drug. Replacement corticosteroids for adrenal insufficiency are permitted.
8. Diabetes mellitus requiring insulin or insulin secretagogue therapy.
9. Poorly controlled diabetes mellitus defined as glycosylated hemoglobin A1c (HbA1c)>7.5% or fasting blood sugar >160 mg/dL.
10. Known impaired cardiac function or clinically significant cardiac disease such as ventricular arrhythmia requiring therapy, congestive heart failure (New York Heart Association Class III or IV), and/or uncontrolled hypertension (defined as systolic blood pressure >150 mmHg and/or diastolic blood pressure >100 mmHg on antihypertensive medications).
11. QTcF interval >470 msec at screening.
12. Myocardial infarction, cardiac stent placement, or unstable angina within 6 months before the first administration of Study Drug.
13. Have clinically significant peripheral vascular disease.
14. Manifestations of malabsorption due to prior gastrointestinal (GI) surgery, GI disease, or for an unknown reason that could alter the absorption of oral medications or Study ISD.
15. Other clinically significant comorbidities, such as uncontrolled pulmonary disease, active central nervous system disease, active infection (any infection requiring systemic therapy), non-healing wounds or injuries, or any other condition that could compromise the subject's participation or confound the interpretation of compliance, adherence, safety, or efficacy results.
16. Pregnant (positive serum pregnancy test), planning to become pregnant during the study, or breastfeeding/planning to breastfeed during the study.
17. Have taken strong CYP3A4 inducers/inhibitors within 7 days before the first administration of serabelisib or have conditions that require the concomitant use of CYP3A4 inducers/inhibitors.
18. Untreated or poorly controlled, gastro-esophageal reflux disease.
19. Have taken histamine-H2 receptor antagonists within 24 hours before the first administration of serabelisib.
20. Have taken proton-pump inhibitors (PPI) within 7 days or 5 half-lives (whichever is the shorter duration) before the first administration of serabelisib or are anticipated to need PPI during the study.
21. Have taken neutralizing antacids within 4 hours before the first administration of serabelisib or are anticipated to need frequent antacid use during the study.
22. Subjects with poorly controlled human immunodeficiency virus (HIV), hepatitis B virus (HBV), and/or hepatitis C virus (HCV) infections.
23. Known allergies to serabelisib excipients or the ISD
   a. Serabelisib excipients: microcrystalline cellulose, low substituted hydroxypropyl cellulose, croscarmellose sodium, colloidal silicon dioxide, and magnesium stearate (non-bovine).
   b. ISD allergens: milk, eggs, fish, Crustacean shellfish, tree nuts, peanuts, wheat, and soybeans.
24. Severe, uncontrolled gout.
25. A BMI <18.5 kg/m², or serious or refractive cachexia or anorexia that, in the Investigator's opinion, realistically prohibits subjects from having energy or appetite sufficient to reliably engage in a strict medical food regimen for an extended time.
26. Any condition that renders the subject unable to satisfactorily chew, swallow, digest, or tolerate (i.e., persistent diarrhea) the majority of foods and liquids of the Study ISD.
27. History of severe nephrolithiasis requiring urologic intervention.
28. Participation in a diet (Atkins, Weight Watchers, Best Life, Nutrisystem, South Beach, Jenny Craig, Paleo Diet, Zone, etc.) or weight loss plan within 10 days prior to the first administration of Study Drug.
29. Severe constipation or condition where exacerbation of constipation is not advisable (e.g., small bowel obstruction history).
30. History of anaphylaxis from food allergy or other disease state requiring avoidance of a particular food, such as celiac disease.
31. Diagnosed eating disorder in the past 10 years.
32. Unwilling to take a non-vegan or non-vegetarian diet.
33. Peripheral neuropathy≥CTC Grade 2

Duration of subject involvement in the study: Subjects may remain on study (expected median=6 months; expected maximum=12 months) until disease progression, unacceptable toxicity, or for other protocol-specified reasons.

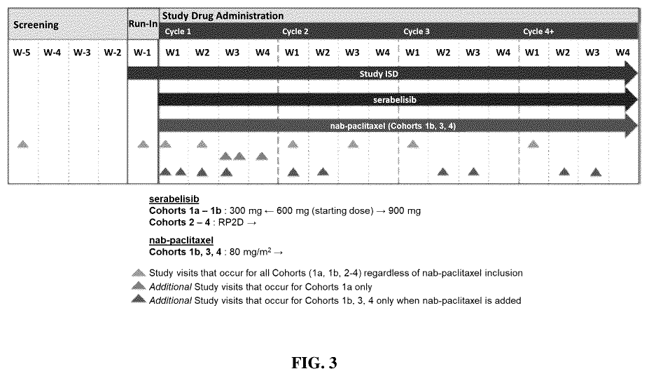

The study schema is illustrated in FIG. 3. Note: Serabelisib doses explored in Cohorts 1a, 1b: starting dose 600 mg 3CDW, escalate to dose 900 mg 3CDW if 600 mg 3CDW is tolerable, de-escalate dose to 300 mg 3CDW if 600 mg 3CDW is not tolerable. 3CDW=3 consecutive days of the week.

TABLE 19, TABLE 20, and TABLE 21 depict the clinical schedules for investigators. TABLE 22, TABLE 23, and TABLE 24 depict the clinical schedules for subjects.

TABLE 19
Cohort 1a Schedule of Activities for Investigators

| | | Intervention Period | | | | | | |
|---|---|---|---|---|---|---|---|---|
| | | | Study Drug Administration | | | | | |
| | | | Cycle 1 | | | | | |
| | SC | RI | W 1 | | W 2 | W 3 | | W 4 |
| | Visit (study day) | | | | | | | |
| | V−1 | V0 | | | V3 | | | |
| | (D −35 | (D −7 or | V1³³ | V2³³ | (D 10) | V3a¹ | V3b¹ | V3c¹ |
| Procedures | to −8) | earlier) | (D 1) | (D 3) | (±1 day) | (D 15) | (D 17) | (D 24) |
| Written informed | X | | | | | | | |
| consent² | | | | | | | | |
| Inclusion and | X | | | | | | | |
| exclusion criteria³ | | | | | | | | |
| Medical history | X | | | | | | | |
| (including | | | | | | | | |
| demographics) | | | | | | | | |
| Physical | X | | X | | | | | |
| examination⁴ | | | | | | | | |
| Height, weight, | X | X⁵ | X^{5, 6} | X⁵ | X⁵ | X⁵ | X⁵ | X⁵ |
| ECOG PS | | | | | | | | |
| Triplicate | X | | X⁸ | X⁸ | X | X⁸ | X | |
| 12-lead ECG | | | | | | | | |
| Vital signs⁷ | X | X | X⁸ | X | X | X | X⁸ | X |
| Continuous glucose | | X⁹ | X¹⁰ | | | X¹⁰ | | |
| monitoring | | | | | | | | |
| (FreeStyle Libre) | | | | | | | | |
| placement/ | | | | | | | | |
| replacement/data | | | | | | | | |
| download | | | | | | | | |
| Dispense | | | X | | | X | | |
| Serabelisib¹² | | | | | | | | |
| Study ISD consumed | | X | X | X | X | X | X | X |
| in clinic¹³ | | | | | | | | |
| Serabelisib PO | | | X | X | X | X | X | X |
| consumed | | | | | | | | |
| (in clinic)¹⁴ | | | | | | | | |
| Adverse events¹⁵ | X (review on an ongoing basis) | | | | | | | |
| Prior and | X | X (review on an ongoing basis) | | | | | | |
| concomitant | | | | | | | | |
| medications¹⁶ | | | | | | | | |
| Study drug | | | | X | X | X | X | X |
| reconciliation¹⁷ | | | | | | | | |
| Virtual visit with | X | X | | X | X | X | | X |
| study dietitian¹⁸ | | | | | | | | |

LABORATORY ASSESSMENTS¹⁹

| | | | | | | | |
|---|---|---|---|---|---|---|---|
| Pregnancy test²⁰ | X | X | | X | X | | X |
| Hematology^{19a} | X | X | | X | X | | X |
| Chemistry^{19b} | X | X | X | X | X | X | X |
| HbA1c | X²² | | | | | | |
| Urinalysis^{19d} | X | X | | X | X | | X |

METABOLIC LABORATORY ASSESSMENTS^{19c}

| | | | | | |
|---|---|---|---|---|---|
| Glucose | X | X | X²¹ | X²¹ | X²¹ |
| Serum insulin | X | X | X²¹ | X²¹ | X²¹ |
| BHB | X | X | X²¹ | X²¹ | X²¹ |
| NEFA | X | X | X²¹ | X²¹ | X²¹ |

PHARMACOKINETIC ASSESSMENTS

| | | | | | |
|---|---|---|---|---|---|
| PK sampling | X²⁴ | X²⁴ | X²⁴ | X²⁴ | X²⁴ |

EFFICACY ASSESSMENTS

| | | |
|---|---|---|
| Circulating | X | X |
| Tumor Markers²⁵ | | |
| Tumor imaging²⁶ | X | |

PHARMACODYNAMIC ASSESSMENTS

| | |
|---|---|
| Tumor | X²⁸ |
| biopsy/collection | |
| of archival tissue | |

EXPLORATORY ASSESSMENTS

| | | | | | |
|---|---|---|---|---|---|
| Buccal cell | | X | X³⁰ | X³⁰ | X³⁰ |
| collection | | | | | |
| PBMC collection³¹ | X | | X | | |

| | Intervention Period | | | | | |
|---|---|---|---|---|---|---|
| | Study Drug Administration | | | | | |
| | | | | Cycle 4 | | End of |
| | Cycle 2 | | Cycle 3 | onward | Unscheduled | Inter- |
| | W 1 | W 3 | W 1 | W 1 | Visit³² | vention |
| | Visit (study day) | | | | | |
| | | | | | | (within 28 |
| | V4 | V5 | V6 | V7 | | days of the |
| Procedures | (D 29) | (D 43) | (D 57) | (D 85+) | | last dose) |
| Written informed | | | | | | |
| consent² | | | | | | |
| Inclusion and | | | | | | |
| exclusion criteria³ | | | | | | |
| Medical history | | | | | | |
| (including | | | | | | |
| demographics) | | | | | | |
| Physical | X | | X | X | X | |
| examination⁴ | | | | | | |
| Height, weight, | X^{5, 6} | X⁵ | X^{5, 6} | X^{5, 6} | X^{5, 6} | X^{5, 6} |
| ECOG PS | | | | | | |
| Triplicate | | | | | X | |
| 12-lead ECG | | | | | | |
| Vital signs⁷ | X | X | X | X | X | X |
| Continuous glucose | X¹⁰ | X¹⁰ | X¹¹ | | | |
| monitoring | | | | | | |
| (FreeStyle Libre) | | | | | | |
| placement/ | | | | | | |
| replacement/data | | | | | | |
| download | | | | | | |
| Dispense | X | | X | X | | |
| Serabelisib¹² | | | | | | |
| Study ISD consumed | X | X | X | X | | |
| in clinic¹³ | | | | | | |
| Serabelisib PO | X | X | X | X | | |
| consumed | | | | | | |
| (in clinic)¹⁴ | | | | | | |
| Adverse events¹⁵ | X (review on an ongoing basis) | | | | | |
| Prior and | X (review on an ongoing basis) | | | | | |
| concomitant | | | | | | |
| medications¹⁶ | | | | | | |
| Study drug | X | X | X | X | | X |
| reconciliation¹⁷ | | | | | | |
| Virtual visit with | X (schedule as needed) | | | | | |
| study dietitian¹⁸ | | | | | | |
| | LABORATORY ASSESSMENTS¹⁹ | | | | | |
| Pregnancy test²⁰ | X | X | X | X | | |
| Hematology^{19a} | X | X | X | X | X | X |
| Chemistry^{19b} | X | X | X | X | X | X |
| HbA1c | | | | X²³ | | X |
| Urinalysis^{19d} | X | X | X | X | X | X |
| | METABOLIC LABORATORY ASSESSMENTS^{19c} | | | | | |
| Glucose | X | | X | X | | X |
| Serum insulin | X | | X | X | | X |
| BHB | X | | X | X | | X |
| NEFA | X | | X | X | | X |
| | PHARMACOKINETIC ASSESSMENTS | | | | | |
| PK sampling | | X²⁴ | | | | |
| | EFFICACY ASSESSMENTS | | | | | |
| Circulating | X | | X | X | | X |
| Tumor Markers²⁵ | | | | | | |
| Tumor imaging²⁶ | | | X²⁷ | | | |
| | PHARMACODYNAMIC ASSESSMENTS | | | | | |
| Tumor | X²⁹ | | | | | |
| biopsy/collection | | | | | | |
| of archival tissue | | | | | | |
| | EXPLORATORY ASSESSMENTS | | | | | |
| Buccal cell | | | | | | |
| collection | | | | | | |
| PBMC collection³¹ | X | | | | | |

¹Visit occurs for Cohort 1 only.
²Written informed consent must be obtained prior to any study-specific Visit −1 (screening) activities.
³Perform a check of inclusion and exclusion criteria at screening; confirm eligibility prior to Visit 0 (run-in). Prior to enrollment of each subject, the site will complete an inclusion/exclusion checklist that will be evaluated in a telephone call with site staff, Sponsor Medical Monitor (optional), and the CRO Medical Monitor. Note that email communication of the checklist will suffice if no telephone call takes place. The sites will communicate the outcome of the meeting and the inclusion policy to the participants.
⁴Physical exam to be completed at the beginning of each subsequent cycle.
⁵Body weight only.
⁶ECOG PS to be completed (and on the first visit of every cycle and upon end of intervention).
⁷Vital signs consist of heart rate, respiratory rate, temperature, and blood pressure.
⁸Vital signs and 12-lead ECG to be performed pre-dose (up to 60 min prior to dosing) and then post-dose at 5 h (±30 min). 12-lead ECG to be performed prior to vital signs and in triplicate after 3 min rest in sitting position.
⁹Insert CGM sensor at clinic. Subjects are provided with two unopened CGM sensors (one for placement during visit and the other to remain unopened and taken home for backup) and education on how to remove sensor, return sensor to clinic, and replace sensor. Should the backup sensor be used at any point, another backup sensor must be provided.
¹⁰The sensor placed at the previous visit will be removed, the data will be downloaded, and a new one placed at least 1 h prior to taking the Study Drug.
¹¹Remove CGM sensor placed at Visit 5. CGM data collection ends upon Visit 6.
¹²Study drug (serabelisib) to be administered PO on three consecutive days per week (either MTW, TWTh, or WThF of each week; subject should be instructed to take Study Drug on the same 3 consecutive days of the week for the entire Study Drug administration). Study Drug will be administered PO in 28-day (4-week; 28 ± 3 days) cycles without a break between cycles. Study Drug will be dispensed in 300 mg tablets and must not be cut, broken, chewed, crushed, mixed with food/liquid before ingestion, or altered in any way. Where Study Drug is not indicated as taken in-clinic, it will be taken at home. For all cohorts, serabelisib will be taken within 15 min of finishing Study ISD meals.
¹³At V0 subjects will bring Study ISD breakfast for consumption in clinic. At visits with pre- and post-Study ISD breakfast/Study Drug assessments, subjects will bring Study ISD breakfast and lunch with them for consumption in clinic (V1, V2, V3, V3a, V3b, V3c). At visits with pre-Study ISD breakfast/Study Drug assessments only (V4, V5, V6, V7+), Investigator and subject should decide together whether the Study ISD breakfast will be consumed in the clinic.
¹⁴Serabelisib to be consumed in clinic within 15 min of finishing Study ISD breakfast (and again within 15 min of finishing Study ISD lunch for V1, V2, V3, V3a, V3b, and V3c,). Subjects will bring their dispensed serabelisib dose with them to clinic visits starting on V2. At visits with pre-Study ISD breakfast/Study Drug assessments only (V4, V5, V6, V7+), Investigator and subject should decide together whether the Study ISD breakfast and Study Drug and will be taken in the clinic.
¹⁵AEs will be collected at Visit 0 (non-related AEs), during the ISD run-in period (non-related and ISD-related), and then after the first administration of serabelisib until end of intervention (non-related, ISD-related, serabelisib-related AEs).
¹⁶Prior and concomitant medications will be collected from 30 days prior to the first administration of Study Drug until end of intervention.
¹⁷Compliance with the dosing schedule will be assessed by reconciliation of the used and unused Study Drug during the clinic visit on Day 1 of each cycle, beginning with Cycle 2.
¹⁸At V0, this will include meal consultation, training with App, etc. Identified schedule of virtual visits is approximate but will occur at least once per week for the first 4 weeks and as needed thereafter.
¹⁹Laboratory assessments to be collected in a fasted state (water consumption encouraged but no food for at least 8 hours prior; assessments not required at V−1 if collected in the past 14 d) and prior to serabelisib dosing include:
^{a}Hematology: CBC with WBC differential (percent and absolute) count, Hb, MCV, MCHC, MCH, RDW, platelet count, and INR (during screening only).
^{b}Chemistry: BUN, creatinine, ALT, AST, ALP, total bilirubin, direct bilirubin, LDH, total protein, sodium, potassium, chloride, bicarbonate, calcium, phosphate, and magnesium.
^{c}Metabolic: blood glucose, serum insulin, triglycerides, total cholesterol, LDL-direct, HDL, BHB and NEFA; triglycerides, total cholesterol, LDL-direct, and HDL are assessed only every other cycle i.e., cycle 2, cycle 4 etc.
^{d}Urinalysis: dipstick protein, glucose, ketones, and blood; pH, specific gravity, nitrite, and leukocyte esterase. A microscopic examination will be performed, if necessary. Urinalysis will also include UACR, b2MG (urinary and/or serum), and NGAL.
²⁰Pregnancy tests for female subjects of child-bearing potential will be done at V−1 during screening (serum only); all subsequent pregnancy tests are to be performed in urine samples and always prior to dosing. Following Cycle 3, a urine pregnancy test must be performed at the beginning of every cycle. All pregnancy tests must confirm that the woman is in a non-pregnant state before dosing.
²¹Serial assessment of glucose, serum insulin, BHB, NEFA: pre- and post-ISD breakfast/Study Drug dosing at 1.5 h (±15 min), 3 h (±30 min), 5 h (±30 min).
²²HbA1c to be collected at screening (V−1) if not measured within 14 d prior to screening.
²³HbA1c to be collected every subsequent 3 months (e.g., Cycle 7, Cycle 10, etc.).
²⁴Serial assessment of serabelisib PK pre-dose and post-dose at 30 min (±15 min), 1 h (±15 min), 1.5 h (±15 min), 3 h (±30 min), and 5 h (±30 min).
²⁵Circulating tumor marker(s) selected by the treating physician (such as CEA, CA 19-9, and CA 125) to be collected at screening and on the first visit of every Study Drug Cycle for the first six months, and once every three Study Drug Cycles thereafter.
²⁶CT scans with/without contrast of chest, abdomen, pelvis.
²⁷CT scans with/without contrast of chest, abdomen, pelvis to be conducted every 8 weeks or as needed following Cycle 3.
²⁸The screening procedures will include evidence by a fresh tumor biopsy or archival tissue confirming mutational status for PIK3CA and PTEN. It is recommended that the most recent archival tumor tissue is sent to the local laboratory for determination of PIK3CA and PTEN mutational status, and PTEN immunohistochemistry. Tumor tissue from a surgical specimen or a biopsy is to be sent to the central laboratory for PD assessment of biomarkers (if only slides are available, then a minimum of 15 slides for surgical specimens and 20 slides for a biopsy specimen, respectively is required; specimens should have been collected from the most recent surgery or biopsy; see the laboratory manual for PD tumor biopsy tissue handling and storage). Results of this procedure confirming PIK3CA and PTEN mutational status as per the protocol eligibility criteria must be done prior to the first administration of Study Drug. For patients who consent to future research, an additional 5 slides from a surgical specimen or biopsy are required.
²⁹Schedule tumor biopsy. Can be performed any time after serabelisib dosing administration on Day 2 (preferred) or 3 of weekly dosing (i.e., Tuesday or Wednesday if dosing started on Monday) or on the day of the visit. If the original lesion identified in screening is not accessible, then another lesion may be chosen. If there is no lesion to biopsy, the subject continues without a biopsy.
³⁰Buccal cells will be collected pre- and 5 h (±30 min) post-serabelisib administration. The collection of buccal cells is only required if the site has the appropriate facilities to collect and process the cells according to the laboratory manual.
³¹PBMCs will be collected at select sites only and will be sent to a central laboratory for exploratory immunophenotype analysis. PBMCs will be collected in a fasted state (water consumption encouraged but no food for at least 8 hours prior) and prior to serabelisib dosing.
³²An unscheduled visit should be performed in the event the subject returns to the site for a visit outside of the scheduled time points or if the subject returns for any follow-up assessments that were not captured in a regular protocol defined visit but are required for this study. The assessments performed by the Investigator during that unscheduled visit will depend on the corresponding follow-up visit but will always include safety assessments.
³³V1 and V2 should not occur on the same day and should be separated by at least 1 day.

TABLE 20
Cohort 2, 3, and 4 Schedule of Activities (no nab-paclitaxel) for Investigators

| | | Intervention Period | | | | | | | | | |
|---|---|---|---|---|---|---|---|---|---|---|---|
| | | | Study Drug Administration Period | | | | | | | | End of |
| | | | Cycle 1 | | | Cycle 2 | | Cycle 3 | Cycle 4+ | Unscheduled | Inter- |
| | SC | RI | W 1 | | W 2 | W 1 | W 3 | W 1 | W 1 | Visit³³ | vention |
| | Visit (study day) | | | | | | | | | | |
| | V −1 | V0 | | | | | | | | | (within 28 |
| | (D −35 | (D −7 or | V1 | V2 | V3 | V4 | V5 | V6 | V7+ | | days of the |
| Procedures | to −8) | earlier) | (D 1) | (D 3) | (D 10) | (D 29) | (D 43) | (D 57) | (D 85+) | | last dose) |
| Written informed | X | | | | | | | | | | |
| consent² | | | | | | | | | | | |
| Inclusion and | X | | | | | | | | | | |
| exclusion criteria³ | | | | | | | | | | | |
| Medical history | X | | | | | | | | | | |
| (including demographics) | | | | | | | | | | | |
| Physical examination | X | | X | | | X | | X | X⁴ | X | |
| Height, weight, ECOG PS | X | X⁵ | X^{5, 6} | X⁵ | X⁵ | X^{5, 6} | X⁵ | X^{5, 6} | X^{5, 6} | X^{5, 6} | X^{5, 6} |
| Triplicate 12-lead ECG | X | | X⁸ | X⁸ | | | | | | X | |
| Vital signs⁷ | X | X | X⁸ | X | X | X | X | X | X | X | X |
| Continuous | | X⁹ | X¹⁰ | | X¹¹ | X¹⁰ | X¹⁰ | X¹² | | | |
| glucose monitoring | | | | | | | | | | | |
| (FreeStyle Libre) | | | | | | | | | | | |
| placement/replacement/ | | | | | | | | | | | |
| data download | | | | | | | | | | | |
| Dispense Serabelisib¹³ | | | X | | | X | | X | X | | |
| Study ISD consumed | | X | X | X | X | X | X | X | | | |
| in clinic¹⁴ | | | | | | | | | | | |
| Serabelisib PO | | | X | X | X | X | X | X | | | |
| consumed (in clinic)¹⁵ | | | | | | | | | | | |
| Adverse events¹⁶ | X (review on an ongoing basis) | | | | | | | | | | |
| Prior and concomitant | X | X (review on an ongoing basis) | | | | | | | | | |
| medications¹⁷ | | | | | | | | | | | |
| Study drug | | | | X | X | X | X | X | X | | X |
| reconciliation¹⁸ | | | | | | | | | | | |

| | | | | | |
|---|---|---|---|---|---|
| Virtual visit with | X | X | X | X | X (schedule as needed) |
| study dietitian¹⁹ | | | | | |

LABORATORY ASSESSMENTS²⁰

| | | | | | | | | | | |
|---|---|---|---|---|---|---|---|---|---|---|
| Pregnancy test²⁰ | X | X | | X | X | X | X | X | | |
| Hematology^{19a} | X | X | | X | X | X | X | X | X | X |
| Chemistry^{19b} | X | X | X | X | X | X | X | X | X | X |
| HbA1c | X²³ | | | | | | | X²⁴ | | X |
| Urinalysis^{19d} | X | X | | X | X | X | X | X | X | X |

METABOLIC LABORATORY ASSESSMENTS^{19c}

| | | |
|---|---|---|
| Glucose | X | X²² |
| Serum insulin | X | X²² |

PHARMACOKINETIC ASSESSMENTS

| | | |
|---|---|---|
| PK sampling | X²⁵ | X²⁵ |

EFFICACY ASSESSMENTS

| | | | | | | |
|---|---|---|---|---|---|---|
| Circulating Tumor | X | X | X | X | X | X |
| Markers²⁶ | | | | | | |
| Tumor imaging²⁷ | X | | | X²⁸ | | |

PHARMACODYNAMIC ASSESSMENTS

| | | |
|---|---|---|
| Tumor | X²⁹ | X³⁰ |
| biopsy/collection of | | |
| archival tissue | | |

EXPLORATORY ASSESSMENTS

| | | | | | |
|---|---|---|---|---|---|
| Buccal cell collection | | X | | X³¹ | |
| PBMC collection³² | X | | X | | X |

1. Written informed consent must be obtained prior to any study-specific Visit −1 (screening) activities.
²Perform a check of inclusion and exclusion criteria at screening; confirm eligibility prior to Visit 0 (run-in). Prior to enrollment of each subject, the site will complete an inclusion/exclusion checklist that will be evaluated in a telephone call with site staff, Sponsor Medical Monitor (optional), and the CRO Medical Monitor. Note that email communication of the checklist will suffice if no telephone call takes place. The sites will communicate the outcome of the meeting and the inclusion policy to the participants.
³Physical exam to be completed at the beginning of each subsequent cycle.
⁴Body weight only.
⁵ECOG PS to be completed (and on the first visit of every cycle and upon end of intervention).
⁶Vital signs consist of heart rate, respiratory rate, temperature, and blood pressure.
⁷Vital signs and 12-lead ECG to be performed pre-dose (up to 60 min prior to dosing) and then post-dose at 5 h (±30 min). 12-lead ECG to be performed prior to vital signs and in triplicate after 3 min rest in sitting position.
⁸Insert CGM sensor at clinic. Subjects are provided with two unopened CGM sensors (one for placement during visit and the other to remain unopened and taken home for backup) and education on how to remove sensor, return sensor to clinic, and replace sensor. Should the backup sensor be used at any point, another backup sensor must be provided.
⁹The sensor placed at the previous visit will be removed, the data will be downloaded, and a new one placed at least 1 h prior to taking the Study Drug.
¹⁰Subjects are provided with another unopened CGM sensor to take home with them. Subjects will receive a reminder phone call on Day 13 to remove the previous sensor, replace it with the new sensor on Day 14 at least 1 h prior to taking Study Drug, and return the used sensor to the clinic upon their next visit.
¹¹Remove CGM sensor placed at Visit 5. CGM data collection ends upon Visit 6.
¹²Study drug (serabelisib) to be administered PO on three consecutive days per week (either MTW, TWTh, or WThF of each week; subject should be instructed to take Study Drug on the same 3 consecutive days of the week for the entire Study Drug administration). Study drug will be administered PO in 28-day (4-week; 28 ± 3 days) cycles without a break between cycles. Study Drug will be dispensed in 300 mg tablets and must not be cut, broken, chewed, crushed, mixed with food/liquid before ingestion, or altered in any way. Where Study Drug is not indicated as taken in-clinic, it will be taken at home. For all cohorts, serabelisib will be taken within 15 min of finishing Study ISD meals.
¹³At V0 subjects will bring Study ISD breakfast for consumption in clinic. At visits with pre- and post-Study ISD breakfast/Study Drug assessments, subjects will bring Study ISD breakfast and lunch with them for consumption in clinic (V1, V3). At visits with pre-Study ISD breakfast/Study Drug assessments only (V4, V5, V6, V7+), Investigator and subject should decide together whether the Study ISD breakfast will be consumed in the clinic.
¹⁴Serabelisib to be consumed in clinic within 15 min of finishing Study ISD breakfast (and again within 15 min of finishing Study ISD lunch V1, V3). Subjects will bring their dispensed serabelisib dose with them to clinic visits starting upon V2. At visits with pre-Study ISD breakfast/Study Drug assessments only (V4, V5, V6, V7+), Investigator and subject should decide together whether the Study ISD breakfast and Study Drug and will be taken in the clinic.
¹⁵AEs will be collected at Visit 0 (non-related AEs), during the ISD run-in period (non-related and ISD-related), and then after the first administration of serabelisib until end of intervention (non-related, ISD-related, and serabelisib-related AEs).
¹⁶Prior and concomitant medications will be collected from 30 days prior to the first administration of Study Drug until end of intervention.
¹⁷Compliance with the dosing schedule will be assessed by reconciliation of the used and unused Study Drug during the clinic visit on Day 1 of each cycle, beginning with Cycle 2.
¹⁸At V0, this will include meal consultation, training with the App, etc. Identified schedule of virtual visits is approximate but will occur at least once per week for the first 4 weeks and as needed thereafter.
¹⁹Laboratory assessments to be collected in a fasted state (water consumption encouraged but no food for at least 8 hours prior; assessments not required at V−1 if collected in the past 14 d) and prior to serabelisib dosing include:
^{a}Hematology: CBC with WBC differential (percent and absolute) count, Hb, MCV, MCHC, MCH, RDW, platelet count and INR (during Screening only).
^{b}Chemistry: BUN, creatinine, ALT, AST, ALP, total bilirubin, direct bilirubin, LDH, total protein, sodium, potassium, chloride, bicarbonate, calcium, phosphate, and magnesium.
^{c}Metabolic: blood glucose, serum insulin, triglycerides, total cholesterol, LDL-direct, HDL; triglycerides, total cholesterol, LDL-direct, and HDL are assessed only once each cycle.
^{d}Urinalysis: dipstick protein, glucose, ketones, and blood; pH, specific gravity, nitrite, and leukocyte esterase. A microscopic examination will be performed, if necessary. Urinalysis will also include UACR, b2MG (urinary and/or serum), and NGAL.
²⁰Pregnancy tests for female subjects of child-bearing potential will be done at V−1 during screening (serum only); all subsequent pregnancy tests are to be performed in urine samples and always prior to dosing. Following Cycle 3, a urine pregnancy test must be performed at the beginning of every cycle. All pregnancy tests must confirm that the woman is in a non-pregnant state before dosing.
²¹Assessment of glucose, serum insulin: pre-Study ISD breakfast/Study Drug dosing.
²²Serial assessment of glucose, serum insulin: pre- Study ISD breakfast/Study Drug dosing and post-dose at 1.5 h (±15 min), 3 h (±30 min), 5 h (±30 min).
²³HbA1c to be collected at screening (V−1) if not measured within 14 d prior to screening.
²⁴HbA1c to be collected every subsequent 3 months (e.g., Cycle 7, Cycle 10, etc.).
²⁵Serial assessment of serabelisib PK pre-dose and post-dose at 30 min (±15 min), 1 h (±15 min), 1.5 h (±15 min), 3 h (±30 min), and 5 h (±30 min).
²⁶Circulating tumor marker(s) selected by the treating physician (such as CEA, CA 19-9, and CA 125) to be collected at screening and on the first visit of every Study Drug Cycle for the first six months, and once every three Study Drug Cycles thereafter.
²⁷CT scans with/without contrast of chest, abdomen, pelvis.
²⁸CT scans with contrast of chest, abdomen, pelvis to be conducted after completion of each even numbered cycle (starting with Cycle 4).
²⁹The screening procedures will include evidence by a fresh tumor biopsy or archival tissue confirming mutational status for PIK3CA and PTEN. It is recommended that the most recent archival tumor tissue is sent to the local laboratory for determination of PIK3CA and PTEN mutational status, and PTEN immunohistochemistry. Tumor tissue from a surgical specimen or a biopsy is to be sent to the central laboratory for PD assessment of biomarkers (if only slides are available, then a minimum of 15 slides for surgical specimens and 20 slides for a biopsy specimen, respectively is required; specimens should have been collected from the most recent surgery or biopsy; see the laboratory manual for PD tumor biopsy tissue handling and storage). Results of this procedure confirming PIK3CA and PTEN mutational status as per the protocol eligibility criteria must be done prior to the first administration of Study Drug. For patients who consent to future research, an additional 5 slides from a surgical specimen or biopsy are required.
³⁰Schedule tumor biopsy. Can be performed any time after serabelisib dosing administration on day 2 (preferred) or 3 of weekly dosing (i.e., Tuesday or Wednesday if dosing started on Monday). If the original lesion identified in screening is not accessible, then another lesion may be chosen. If there is no lesion to biopsy, the subject continues without a biopsy.
³¹Buccal cells will be collected pre- and 5 h (±30 min) post-serabelisib administration. The collection of buccal cells is only required if the site has the appropriate facilities to collect and process the cells according to the laboratory manual.
³²PBMCs will be collected at select sites only and will be sent to a central laboratory for exploratory immunophenotype analysis. PBMC's will be collected in a fasted state (water consumption encouraged but no food for at least 8 hours prior) and prior to serabelisib dosing.
An unscheduled visit should be added in the event the subject returns to the site for a visit outside of the scheduled time points or if the subject returns for any follow-up assessments that were not captured in a regular protocol defined visit but are required for this study. The assessments performed by the Investigator during that unscheduled visit will depend on the corresponding follow-up visit but will always include safety assessments.

TABLE 21
Cohort 1b, 3, and 4 Schedule of Activities (with nab-paclitaxel) for Investigators

| | | Intervention Period | | | | | | | |
|---|---|---|---|---|---|---|---|---|---|
| | | | Study Drug Administration Period | | | | | | |
| | | | Cycle 1 | | | | | | Cycle 2 |
| | SC | RI | W 1 | | | W 2 | | W 3 | W 1 |
| | Visit (study day) | | | | | | | | |
| | V−1 | V0 | | | | | | | |
| | (D −35 | (D −7 or | V1 | V1a | V2 | V3 | V3d | V4 | V5 |
| Procedures | to −8) | earlier) | (D 1) | (D 2) | (D 4) | (D 8) | (D 10) | (D 15) | (D 29) |
| Written informed | X | | | | | | | | |
| consent¹ | | | | | | | | | |
| Inclusion and | X | | | | | | | | |
| exclusion | | | | | | | | | |
| criteria² | | | | | | | | | |
| Medical history | X | | | | | | | | |
| (including | | | | | | | | | |
| demographics) | | | | | | | | | |
| Physical examination | X | | X | X | | X | | X | X |
| Height, weight, | X | X⁴ | X^{4, 5} | | | X⁴ | X⁴ | X^{4, 5} | X^{4, 5} |
| ECOG PS | | | | | | | | | |
| Triplicate 12- | X | | X⁷ | | | X | | X⁷ | |
| lead ECG | | | | | | | | | |
| Vital signs⁷ | X | X | X⁷ | X⁷ | | X | X | X⁷ | X |
| Continuous | | X⁸ | | X⁹ | | | X¹⁰ | | |
| glucose monitoring | | | | | | | | | |
| (FreeStyle Libre) | | | | | | | | | |
| placement/ | | | | | | | | | |
| replacement/data | | | | | | | | | |
| download | | | | | | | | | |
| Nab-paclitaxel | | | X | | | X | | X | X |
| Dispense | | | | X | | | | | |
| Serabelisib¹² | | | | | | | | | |
| Study ISD consumed | | X | | X | | | X | | |
| in clinic¹³ | | | | | | | | | |
| Serabelisib PO | | | | X | | | X | | |
| consumed | | | | | | | | | |
| (in clinic)¹⁴ | | | | | | | | | |
| Adverse events¹⁵ | X (review on an ongoing basis) | | | | | | | | |
| Prior and | X | X (review on an ongoing basis) | | | | | | | |
| concomitant | | | | | | | | | |
| medications¹⁶ | | | | | | | | | |
| Study drug | | | | | | X | X | X | X |
| reconciliation¹⁷ | | | | | | | | | |
| Virtual visit with | X | X | | | X | X | X | X | X (schedule as needed) |
| study dietitian¹⁸ | | | | | | | | | |

LABORATORY ASSESSMENTS²⁰

| | | | | | | | |
|---|---|---|---|---|---|---|---|
| Pregnancy test¹⁹ | X | X | | X | | X | X |
| Hematology^{20a} | X | X | | X | | X | X |
| Chemistry^{20b} | X | X | | X | | X | X |
| HbA1c | X²³ | | | | | | |
| Urinalysis^{20d} | X | | X | | X | | X |

METABOLIC LABORATORY ASSESSMENTS (Cohort 1b)^{20c}

| | | | | |
|---|---|---|---|---|
| Glucose | X²¹ | X²¹ | X²² | X²² |
| Serum insulin | X²¹ | X²¹ | X²² | X²² |
| BHB | X²¹ | X²¹ | X²² | X²² |
| NEFA | X²¹ | X²¹ | X²² | X²² |

METABOLIC LABORATORY ASSESSMENTS (Cohorts 3 and 4)

| | | |
|---|---|---|
| Glucose | X²¹ | X²² |
| Serum insulin | X²¹ | X²² |

PHARMACOKINETIC ASSESSMENTS

| | | |
|---|---|---|
| PK sampling | X²⁵ | X²⁵ |

EFFICACY ASSESSMENTS

| | | | |
|---|---|---|---|
| Circulating | X | X | X |
| Tumor Markers²⁶ | | | |
| Tumor imaging²⁷ | X | | |

PHARMACODYNAMIC ASSESSMENTS

| | | |
|---|---|---|
| Tumor | X²⁹ | X³⁰ |
| biopsy/collection | | |
| of archival tissue | | |

EXPLORATORY ASSESSMENTS

| | | | | | |
|---|---|---|---|---|---|
| Buccal cell collection | | X | | X³¹ | |
| PBMC collection³² | X | | X | | X |

| | Intervention Period | | | | | |
|---|---|---|---|---|---|---|
| | Study Drug Administration Period | | | | | End of |
| | Cycle 2 | | | Cycle 3+ | Unscheduled | Inter- |
| | W 1 | W 2 | W 3 | W 1-4 | Visit³³ | vention |
| | Visit (study day) | | | | | |
| | | | | Once | | (within 28 |
| | V5a | V6 | V7+ | weekly | | days of the |
| Procedures | (D 30) | (D 36) | (D 43) | visits | | last dose) |
| Written informed | | | | | | |
| consent¹ | | | | | | |
| Inclusion and | | | | | | |
| exclusion | | | | | | |
| criteria² | | | | | | |
| Medical history | | | | | | |
| (including | | | | | | |
| demographics) | | | | | | |
| Physical examination | | X | | X³ | X | |
| Height, weight, | | X^{4, 5} | | X^{4, 5} | X^{4, 5} | X^{4, 5} |
| ECOG PS | | | | | | |
| Triplicate 12- | | | | X⁸ | X | |
| lead ECG | | | | | | |
| Vital signs⁷ | X | X | | X | X | X |
| Continuous | X⁹ | | X¹¹ | | | |
| glucose monitoring | | | | | | |
| (FreeStyle Libre) | | | | | | |
| placement/ | | | | | | |
| replacement/data | | | | | | |
| download | | | | | | |
| Nab-paclitaxel | | X | X | X | | |
| Dispense | X | | | X | | |
| Serabelisib¹² | | | | | | |
| Study ISD consumed | | | | | | |
| in clinic¹³ | | | | | | |
| Serabelisib PO | | | | | | |
| consumed | | | | | | |
| (in clinic)¹⁴ | | | | | | |
| Adverse events¹⁵ | X (review on an ongoing basis) | | | | | |
| Prior and | X (review on an ongoing basis) | | | | | |
| concomitant | | | | | | |
| medications¹⁶ | | | | | | |
| Study drug | X | X | X | X | | X |
| reconciliation¹⁷ | | | | | | |
| Virtual visit with | X (schedule as needed) | | | | | |
| study dietitian¹⁸ | | | | | | |
| | LABORATORY ASSESSMENTS²⁰ | | | | | |
| Pregnancy test¹⁹ | | X | X | X | | |
| Hematology^{20a} | | X | X | X | X | X |
| Chemistry^{20b} | | X | X | X | X | X |
| HbA1c | X | | | X²⁴ | | X |
| Urinalysis^{20d} | | X | | X | X | X |
| | METABOLIC LABORATORY ASSESSMENTS (Cohort 1b)^{20c} | | | | | |
| Glucose | X²¹ | | | | | X |
| Serum insulin | X²¹ | | | | | X |
| BHB | X²¹ | | | | | X |
| NEFA | X²¹ | | | | | X |
| | METABOLIC LABORATORY ASSESSMENTS (Cohorts 3 and 4) | | | | | |
| Glucose | | | | | | |
| Serum insulin | | | | | | |
| | PHARMACOKINETIC ASSESSMENTS | | | | | |
| PK sampling | | | | | | |
| | EFFICACY ASSESSMENTS | | | | | |
| Circulating | | X | | X | | X |
| Tumor Markers²⁶ | | | | | | |
| Tumor imaging²⁷ | | X²⁸ | | X²⁸ | | |
| | PHARMACODYNAMIC ASSESSMENTS | | | | | |
| Tumor | | | | | | |
| biopsy/collection | | | | | | |
| of archival tissue | | | | | | |
| | EXPLORATORY ASSESSMENTS | | | | | |
| Buccal cell | | | | | | |
| collection | | | | | | |
| PBMC | | | | | | |
| collection³² | | | | | | |

¹Written informed consent must be obtained prior to any study-specific Visit −1 (screening) activities.
²Perform a check of inclusion and exclusion criteria at screening; confirm eligibility prior to Visit 0 (run-in). Prior to enrollment of each subject, the site will complete an inclusion/exclusion checklist that will be evaluated in a telephone call with site staff, Sponsor Medical Monitor (optional), and the CRO Medical Monitor. Note that email communication of the checklist will suffice if no telephone call takes place. The sites will communicate the outcome of the meeting and the inclusion policy to the participants.
³Physical exam to be completed at the beginning of each subsequent cycle.
⁴Body weight only.
⁵ECOG PS to be completed (and on the first visit of every cycle and upon end of intervention).
6. Vital signs consist of heart rate, respiratory rate, temperature, and blood pressure.
⁷Vital signs and 12-lead ECG (in triplicate) to be performed pre-dose (up to 60 min prior to dosing) and then post-dose at 5 h (±30 min). 12-lead ECG to be performed prior to vital signs and in triplicate after 3 min rest in sitting position.
⁸Insert CGM sensor at clinic. Subjects are provided with two unopened CGM sensors (one for placement during visit and the other to remain unopened and taken home for backup) and education on how to remove sensor, return sensor to clinic, and replace sensor. Should the backup sensor be used at any point, another backup sensor must be provided.
⁹The sensor placed at the previous visit will be removed, the data will be downloaded, and a new one placed at least 1 h prior to taking the Study Drug.
¹⁰Subjects are provided with another unopened CGM sensor to take home with them. Subjects will receive a reminder phone call on Day 13 to remove the previous sensor, replace it with the new sensor on Day 14 at least 1 h prior to taking Study Drug, and return the used sensor to the clinic upon their next visit.
¹¹Remove CGM sensor placed at Visit 5. CGM data collection ends upon Visit 6.
¹²Study drug (serabelisib) to be administered PO on three consecutive days per week (either MTW, TWTh, or WThF of each week; subject should be instructed to take Study Drug on the same 3 consecutive days of the week for the entire Study Drug administration). Study drug will be administered PO in 28-day (4-week; 28 ± 3 days) cycles without a break between cycles. Study Drug will be dispensed in 300 mg tablets and must not be cut, broken, chewed, crushed, mixed with food/liquid before ingestion, or altered in any way. Where Study Drug is not indicated as taken in-clinic, it will be taken at home. For all cohorts, serabelisib will be taken within 15 min of finishing Study ISD meals.
¹³At V0 subjects will bring Study ISD breakfast for consumption in clinic. At visits with pre- and post-Study ISD breakfast/Study Drug assessments, subjects will bring Study ISD breakfast and lunch with them for consumption in clinic (V1a, V3d). At visits with pre-Study ISD breakfast/Study Drug assessments only (V5a+), Investigator and subject should decide together whether the Study ISD breakfast will be consumed in the clinic.
¹⁴Serabelisib to be consumed in clinic within 15 min of finishing Study ISD breakfast (and again within 15 min of finishing Study ISD lunch V1a, V3d). Subjects will bring their dispensed serabelisib dose with them to clinic visits starting upon V2. At visits with pre-Study ISD breakfast/Study Drug assessments only (V5a+), Investigator and subject should decide together whether the Study ISD breakfast and Study Drug and will be taken in the clinic.
¹⁵AEs will be collected at Visit 0 (non-related AEs), during the ISD run-in period (non-related and ISD-related), and then after the first administration of serabelisib until end of intervention (non-related, ISD-related, and serabelisib-related AEs).
¹⁶Prior and concomitant medications will be collected from 30 days prior to the first administration of Study Drug until end of intervention.
¹⁷Compliance with the dosing schedule will be assessed by reconciliation of the used and unused Study Drug during the clinic visit on Day 1 of each cycle, beginning with Cycle 2.
¹⁸At V0, this will include meal consultation, training with App, etc. Identified schedule of virtual visits is approximate but will occur at least once per week for the first 4 weeks and as needed thereafter.
¹⁹Pregnancy tests for female subjects of child-bearing potential will be done at V−1 during screening (serum only); all subsequent pregnancy tests are to be performed in urine samples and always prior to dosing. Following Cycle 3, a urine pregnancy test must be performed at the beginning of every cycle. All pregnancy tests must confirm that the woman is in a non-pregnant state before dosing.
²⁰Laboratory assessments to be collected in a fasted state (water consumption encouraged but no food for at least 8 hours prior; assessments not required at V−1 if collected in the past 14 d) and prior to serabelisib dosing include:
^{a}Hematology: CBC with WBC differential (percent and absolute) count, Hb, MCV, MCHC, MCH, RDW, platelet count and INR (during Screening only).
^{b}Chemistry: BUN, creatinine, ALT, AST, ALP, total bilirubin, direct bilirubin, LDH, total protein, sodium, potassium, chloride, bicarbonate, calcium, phosphate, and magnesium.
^{c}Metabolic: blood glucose, serum insulin, triglycerides, total cholesterol, LDL-direct, HDL, NEFA, and BHB;
^{d}Urinalysis: dipstick protein, glucose, ketones, and blood; pH, specific gravity, nitrite, and leukocyte esterase. A microscopic examination will be performed, if necessary. Urinalysis will also include UACR, b2MG (urinary and/or serum), and NGAL.
²¹For Cohort 1b, assessment of glucose, serum insulin, BHB, NEFA: pre-Study ISD breakfast/Study Drug dosing. Cohorts 3 and 4, assessment of glucose, serum insulin: pre-Study ISD breakfast/Study Drug dosing.
²²For Cohort 1b, serial assessment of glucose, serum insulin, BHB, NEFA: pre- Study ISD breakfast/Study Drug dosing and post-dose at 1.5 h (±15 min), 3 h (±30 min), 5 h (±30 min). For Cohorts 3 and 4, serial assessment of glucose, serum insulin: pre- Study ISD breakfast/Study Drug dosing and post-dose at 1.5 h (±15 min), 3 h (±30 min), 5 h (±30 min) on Visit 3d (D 10) only.
²³HbA1c to be collected at screening (V−1) if not measured within 14 d prior to screening.
²⁴HbA1c to be collected every subsequent 3 months (e.g., Cycle 7, Cycle 10, etc.).
²⁵Serial assessment of serabelisib PK pre-dose and post-dose at 30 min (±15 min), 1 h (±15 min), 1.5 h (±15 min), 3 h (±30 min), and 5 h (±30 min).
²⁶Circulating tumor marker(s) selected by the treating physician (such as CEA, CA 19-9, and CA 125) to be collected at screening and on the first visit of every Study Drug Cycle for the first six months, and once every three Study Drug Cycles thereafter.
²⁷CT scans with/without contrast of chest, abdomen, pelvis.
²⁸CT scans with contrast of chest, abdomen, pelvis to be conducted after completion of each even numbered cycle (starting with Cycle 4).
²⁹The screening procedures will include evidence by a fresh tumor biopsy or archival tissue confirming mutational status for PIK3CA and PTEN. It is recommended that the most recent archival tumor tissue is sent to the local laboratory for determination of PIK3CA and PTEN mutational status, and PTEN immunohistochemistry. Tumor tissue from a surgical specimen or a biopsy is to be sent to the central laboratory for PD assessment of biomarkers (if only slides are available, then a minimum of 15 slides for surgical specimens and 20 slides for a biopsy specimen, respectively is required; specimens should have been collected from the most recent surgery or biopsy; see the laboratory manual for PD tumor biopsy tissue handling and storage). Results of this procedure confirming PIK3CA and PTEN mutational status as per the protocol eligibility criteria must be done prior to the first administration of Study Drug. For patients who consent to future research, an additional 5 slides from a surgical specimen or biopsy are required.
³⁰Schedule tumor biopsy. Can be performed any time after serabelisib dosing administration on day 2 (preferred) or 3 of weekly dosing (i.e., Tuesday or Wednesday if dosing started on Monday). If the original lesion identified in screening is not accessible, then another lesion may be chosen. If there is no lesion to biopsy, the subject continues without a biopsy.
³¹Buccal cells will be collected pre- and 5 h (±30 min) post-serabelisib administration. The collection of buccal cells is only required if the site has the appropriate facilities to collect and process the cells according to the laboratory manual.
³²PBMCs will be collected at select sites only and will be sent to a central laboratory for exploratory immunophenotype analysis. PBMC's will be collected in a fasted state (water consumption encouraged but no food for at least 8 hours prior) and prior to serabelisib dosing.
³³An unscheduled visit should be added in the event the subject returns to the site for a visit outside of the scheduled time points or if the subject returns for any follow-up assessments that were not captured in a regular protocol defined visit but are required for this study. The assessments performed by the Investigator during that unscheduled visit will depend on the corresponding follow-up visit but will always include safety assessments.
³⁴Serabelisib should be dispensed at the beginning of every cycle and as needed.

TABLE 22
Cohort 1a Schedule of Activities for Subjects

| | | Intervention Period | | | | | |
|---|---|---|---|---|---|---|---|
| | | | Study Drug Administration Period | | | | |
| | | | Cycle 1 | | | | |
| | SCR | RI | W 1 | | W 2 | W 3 | |
| | Visit (study day) | | | | | | |
| | V−1 | V0 | V1 | V2 | V3 | V3a | V3b |
| | (D −35 | (D −7 or | (D 1) | (D 3) | (D 10) | (D 15) | (D 17) |
| Procedures | to −8) | earlier) | (±1 day) | (±1 day) | (±1 day) | (±1 day) | (±1 day) |
| Virtual visit | | X | | X (scheduled as needed) | | | |
| with dietitian | | | | | | | |
| (meal | | | | | | | |
| consultation, | | | | | | | |
| training with | | | | | | | |
| App, etc.) | | | | | | | |
| Bring smart | | X | X | X | X | X | X |
| phone or tablet | | | | | | | |
| to clinic visit | | | | | | | |
| Arrive at | X | X | X | X | X | X | X |
| clinic fasted¹ | | | | | | | |
| Bring breakfast | | X | X | X | X | X | X |
| from Study ISD | | | | | | | |
| menu to clinic² | | | | | | | |
| Bring dispensed | | | | X³ | X³ | X³ | X³ |
| serabelisib | | | | | | | |
| dose to clinic | | | | | | | |

| Fasted morning | X (daily for 2 weeks and then as directed by the study dietitian)⁴ | |
|---|---|---|
| BHB (via finger | | |
| stick); report | | |
| via App | | |
| Report | X (daily on an ongoing basis) | |
| administration | | |
| of and | | |
| satisfaction | | |
| with Study ISD | | |
| via App | | |
| Report body | X (weekly on an ongoing basis) | |
| weight from | | |
| at-home scale | | |
| via App | | |
| Report at-home | | X (daily on an ongoing basis) |
| administration | | |
| of serabelisib | | |
| via App | | |

| | Intervention Period | | | | | |
|---|---|---|---|---|---|---|
| | Study Drug Administration Period | | | | | End of |
| | Cycle 1 | Cycle 2 | | Cycle 3 | Cycle 4+ | Inter- |
| | W 4 | W 1 | W 3 | W 1 | W 1 | vention |
| | Visit (study day) | | | | | |
| | V3c | V4 | V5 | V6 | V7+ | (within 28 |
| | (D 24) | (D 29) | (D 43) | (D 57) | (D 85+) | days of |
| Procedures | (±1 day) | (±3 days) | (±3 days) | (±3 days) | (±3 days) | last dose) |
| Virtual visit | X (scheduled as needed) | | | | | |
| with dietitian | | | | | | |
| (meal | | | | | | |
| consultation, | | | | | | |
| training with | | | | | | |
| App, etc.) | | | | | | |
| Bring smart | X | X | X | X | X | |
| phone or tablet | | | | | | |
| to clinic visit | | | | | | |
| Arrive at | X | X | X | X | X | X |
| clinic fasted¹ | | | | | | |
| Bring breakfast | X | X | X | X | X | |
| from Study ISD | | | | | | |
| menu to clinic² | | | | | | |
| Bring dispensed | X³ | X³ | X³ | X³ | X³ | |
| serabelisib | | | | | | |
| dose to clinic | | | | | | |

| | |
|---|---|
| Fasted morning | X (daily for 2 weeks and then as directed by the study dietitian)⁴ |
| BHB (via finger | |
| stick); report | |
| via App | |
| Report | X (daily on an ongoing basis) |
| administration | |
| of and | |
| satisfaction | |
| with Study ISD | |
| via App | |
| Report body | X (weekly on an ongoing basis) |
| weight from | |
| at-home scale | |
| via App | |
| Report at-home | X (daily on an ongoing basis) |
| administration | |
| of serabelisib | |
| via App | |

¹Water consumption encouraged but no food for at least 8 hours prior.
²At V0 subjects will bring Study ISD breakfast for consumption in clinic. At visits with pre- and post-Study ISD breakfast/Study Drug assessments, subjects will bring Study ISD breakfast and lunch with them for consumption in clinic (V1, V2, V3, V3a, V3b, V3c). At visits with pre-Study ISD breakfast/Study Drug assessments only (V4, V5, V6, V7+), Investigator and subject should decide together whether the Study ISD breakfast will be consumed in the clinic.
³Serabelisib will be self-administered PO within 15 min of finishing Study ISD meals on three consecutive days of every week starting with either Monday, Tuesday, or Wednesday for the remainder of the trial. At visits with pre-Study ISD breakfast/Study Drug assessments only (V4, V5, V6, V7+), Investigator and subject should decide together whether the Study ISD breakfast and Study Drug will be consumed in the clinic.
⁴Fasted morning BHB measurements will occur daily for two weeks upon run-in (i.e., starting on V0 and through Week 1 Cycle 1 of serabelisib dosing) and intermittently thereafter, as directed by the study dietitian.

TABLE 23
Cohorts 2, 3, and 4 Schedule of Activities (no nab-paclitaxel) for Subjects

| | | Intervention Period | | | | | | | | |
|---|---|---|---|---|---|---|---|---|---|---|
| | | | Study Drug Administration Period | | | | | | | End of |
| | | | Cycle 1 | | | Cycle 2 | | Cycle 3 | Cycles 4+ | Inter- |
| | SCR | RI | W 1 | | W 2 | W 1 | W 3¹ | W 1 | W 1 | vention |
| | Visit (study day) | | | | | | | | | |
| | V−1 | V0 | V1 | V2 | V3 | V4 | V5 | V6 | V7+ | (within 28 |
| | (D −35 | (D −7 or | (D 1) | (D 3) | (D 10) | (D 29) | (D 43) | (D 57) | (D 85+) | days of |
| Procedures | to −8) | earlier) | (±1 day) | (±1 day) | (±1 day) | (±3 days) | (±3 days) | (±3 days) | (±3 days) | last dose) |
| Virtual visit with | | X | | X | X (scheduled as needed) | | | | | |
| study dietitian (meal | | | | | | | | | | |
| consultation, training | | | | | | | | | | |
| with App, etc.) | | | | | | | | | | |
| Bring smart phone or | | X | X | X | X | X | X | X | X | |
| tablet to clinic visit | | | | | | | | | | |
| Arrive at clinic fasted¹ | X | X | X | X | X | X | X | X | X | X |
| Bring breakfast from | | X | X | X | X | X | X | X | X | |
| Study ISD menu to | | | | | | | | | | |
| clinic² | | | | | | | | | | |
| Bring dispensed | | | | X³ | X³ | X³ | X³ | X³ | X³ | |
| serabelisib dose | | | | | | | | | | |
| to clinic | | | | | | | | | | |

| | |
|---|---|
| Fasted morning BHB | X (daily on an ongoing basis)⁴ |
| (via finger stick); | |
| report via App | |
| Report administration | X (daily on an ongoing basis) |
| of and satisfaction | |
| with Study ISD via App | |
| Report body weight from | X (weekly on an ongoing basis) |
| at-home scale via App | |
| Report at-home | X (daily on an ongoing basis) |
| administration of | |
| serabelisib via App | |

¹Water consumption encouraged but no food for at least 8 hours prior.
²At V0 subjects will bring Study ISD breakfast for consumption in clinic. At visits with pre- and post-Study ISD breakfast/Study Drug assessments, subjects will bring Study ISD breakfast and lunch with them for consumption in clinic (V1, V2, V3). At visits with pre-Study ISD breakfast/Study Drug assessments only (V4, V5, V6, V7+), Investigator and subject should decide together whether the Study ISD breakfast will be consumed in the clinic.
³Serabelisib will be self-administered PO within 15 min of finishing Study ISD meals on three consecutive days of every week starting with either Monday, Tuesday, or Wednesday for the remainder of the trial. At visits with pre-Study ISD breakfast/Study Drug assessments only (V4, V5, V6, V7+), Investigator and subject should decide together whether the Study ISD breakfast and Study ISD Drug will be consumed in the clinic.
⁴Fasted morning BHB measurements will occur daily for two weeks upon run-in (i.e., starting on V0 and through Week 1 Cycle 1 of serabelisib dosing) and intermittently thereafter, as directed by the study dietitian.

TABLE 24
Cohorts 1b, 3, and 4 Schedule of Activities (with nab-paclitaxel) for Subjects

| | | Intervention Period | | | | | | |
|---|---|---|---|---|---|---|---|---|
| | | | Study Drug Administration Period | | | | | |
| | | | Cycle 1 | | | | | |
| | SCR | RI | W 1 | | | W 2 | W 3 | |
| | Visit (study day) | | | | | | | |
| | V−1 | V0 | V1 | V1a | V2 | V3 | V3d | V4 |
| | (D −28 | (D −7 or | (D 1) | (D 2) | (D 4) | (D 8) | (D 10) | (D 10) |
| Procedures | to −8) | earlier) | (±1 day) | (±1 day) | (±1 day) | (±1 days) | (±1 day) | (±3 days) |
| Virtual visit | | X | | X (scheduled as needed) | | | | |
| with study | | | | | | | | |
| dietitian | | | | | | | | |
| (meal | | | | | | | | |
| consultation, | | | | | | | | |
| training with | | | | | | | | |
| App, etc.) | | | | | | | | |
| Bring smart | | X | X | X | | X | X | X |
| phone or | | | | | | | | |
| tablet to | | | | | | | | |
| clinic visit | | | | | | | | |
| Arrive at | X | X | X | X | | X | X | X |
| clinic fasted¹ | | | | | | | | |
| Bring breakfast | | X | | X | | | X | |
| from Study ISD | | | | | | | | |
| menu to clinic² | | | | | | | | |
| Bring dispensed | | | | | | | X³ | |
| serabelisib | | | | | | | | |
| dose to clinic | | | | | | | | |

| Fasted morning | X (daily on an ongoing basis)⁴ | |
|---|---|---|
| BHB (via finger | | |
| stick); report | | |
| via App | | |
| Report | X (daily on an ongoing basis) | |
| administration | | |
| of and | | |
| satisfaction | | |
| with Study ISD | | |
| via App | | |
| Report body | X (weekly on an ongoing basis) | |
| weight from | | |
| at-home scale | | |
| via App | | |
| Report at-home | | X (daily on an ongoing basis) |
| administration | | |
| of serabelisib | | |
| via App | | |

| | Intervention Period | | | | | |
|---|---|---|---|---|---|---|
| | Study Drug Administration Period | | | | | End of |
| | Cycle 2 | | | | Cycle 3+ | Inter- |
| | W 1 | | W 2 | W 3 | W 1 | vention |
| | Visit (study day) | | | | | |
| | V5 | V5a | V6 | V7 | V8+ | (within 28 |
| | (D 29) | (D 30) | (D 36) | (D 43) | (D 57) | days of |
| Procedures | (±3 days) | (±1 day) | (±3 days) | (±3 days) | (±3 days) | last dose) |
| Virtual visit | X (scheduled as needed) | | | | | |
| with study | | | | | | |
| dietitian | | | | | | |
| (meal | | | | | | |
| consultation, | | | | | | |
| training with | | | | | | |
| App, etc.) | | | | | | |
| Bring smart | X | X | X | X | X | |
| phone or | | | | | | |
| tablet to | | | | | | |
| clinic visit | | | | | | |
| Arrive at | X | X | X | X | X | X |
| clinic fasted¹ | | | | | | |
| Bring breakfast | | | | | | |
| from Study ISD | | | | | | |
| menu to clinic² | | | | | | |
| Bring dispensed | | | | | | |
| serabelisib | | | | | | |
| dose to clinic | | | | | | |

| | |
|---|---|
| Fasted morning | X (daily on an ongoing basis)⁴ |
| BHB (via finger | |
| stick); report | |
| via App | |
| Report | X (daily on an ongoing basis) |
| administration | |
| of and | |
| satisfaction | |
| with Study ISD | |
| via App | |
| Report body | X (weekly on an ongoing basis) |
| weight from | |
| at-home scale | |
| via App | |
| Report at-home | X (daily on an ongoing basis) |
| administration | |
| of serabelisib | |
| via App | |

¹Water consumption encouraged but no food for at least 8 hours prior.
²At V0 subjects will bring Study ISD breakfast for consumption in clinic. At visits with pre- and post-Study ISD breakfast/Study Drug assessments, subjects will bring Study ISD breakfast and lunch with them for consumption in clinic (V1a, V3d). At visits with pre-Study ISD breakfast/Study Drug assessments only (V5a+), Investigator and subject should decide together whether the Study ISD breakfast will be consumed in the clinic.
³Serabelisib will be self-administered PO within 15 min of finishing Study ISD meals on three consecutive days of every week starting with either Monday, Tuesday, or Wednesday for the remainder of the trial. At visits with pre-Study ISD breakfast/Study Drug assessments only (V5a+), Investigator and subject should decide together whether the Study ISD breakfast and Study ISD Drug will be consumed in the clinic.
⁴Fasted morning BHB measurements will occur daily for two weeks upon run-in (i.e., starting on V0 and through Week 1 Cycle 1 of serabelisib dosing) and intermittently thereafter, as directed by the study dietitian.

Dose Modification Strategy (Cohorts 1a, 1b): Dietary fat content has beneficial effects on the PK and GI tolerability of serabelisib. Therefore, an intrasubject dose-modification scheme (Cohort 1a; n=10; Cohort 1b; n=10) will be employed in evaluable subjects with any solid tumor (Cohort 1a) or endometrial cancer (EC) or ovarian clear cell or ovarian endometroid carcinoma (Cohort 1b) with PIK3CA mutation with or without PTEN loss to determine the safety, feasibility, and PK impact of intrasubject modulated dosing for serabelisib in combination with the Study ISD (Cohort 1a) and, in addition, with weekly nab-paclitaxel (Cohort 1b) to derive the optimal dosing regimen for the cohort expansion. Subjects will consume the Study ISD for at least 7 days (run-in period) before taking their first dose of serabelisib to allow for adjustment to the dietary change and assess the metabolic effects, tolerability, and adherence of the Study ISD prior to the first administration of serabelisib and remaining study procedures.

To be conservative, the starting dose of serabelisib in both Cohorts 1a and 1b (TABLE 18) will be 600 mg delivered as 300 mg BID on the same three consecutive days of each week (3CDW; which must start on either a Monday, Tuesday, or Wednesday), which is less than the previously RP2D of 900 mg QD administered PO 3CDW. Serabelisib doses must be consumed within 15 minutes of finishing the Study ISD meal.

Nab-paclitaxel will be administered intravenously weekly according to current prescribing information. The starting dose level of nab-paclitaxel in this study will be 80 mg/m² administered intravenously over 30 minutes on Days 1, 8, and 15 of each 28-day cycle (3-weeks-on/1-week-off schedule).

The pharmacokinetics of serabelisib will be assessed during the two weeks of the 600 mg dosing. Pharmacokinetics will also be assessed following one week of the 600 mg dosing. Following two weeks of 600 mg, a decision will be made by the Investigator and the study team based on safety and tolerability to escalate the dose to 900 mg PO delivered as 300 mg TID 3CDW and consumed within 15 minutes of finishing the Study ISD meal. PK will again be assessed following one week of the 900 mg dosing (TABLE 19). Weekly nab-paclitaxel will be added to the treatment regimen in Cohorts 3 and 4 if results from Cohort 1b indicate a favorable risk-benefit ratio for this regimen.

Cohorts 2, 3, and 4 (Cohort Expansion): Cohort expansion will include three groups of subjects with advanced cancer to evaluate efficacy and confirm the safety, tolerability, and compliance of the combination of the Study ISD and serabelisib RP2D identified in dose modification (Cohort 1). The expansion cohorts include subjects with solid tumors that have failed standard-of-care (SOC) therapy and possess PIK3CA-activating mutations with or without PTEN loss: colorectal cancer (CRC) (Cohort 2), endometrial cancer (EC) (Cohort 3), and ovarian clear cell or ovarian endometrioid carcinoma (Cohort 4). Subjects will consume the Study ISD for at least 7 days (run-in period) before taking their first dose of serabelisib, to allow for adjustment to the dietary change and assess the metabolic effects, tolerability, and adherence of the Study ISD prior to the first administration of serabelisib and remaining study procedures.

FIG. 3 depicts a flowchart of the study using serabelisib in combination with an insulin suppressing diet with nab-paclitaxel in subjects with cancer. The screening is run from 35 days (Week −5) to 8 days prior to the first planned administration of serabelisib. The run-in is from at least 7 days to 1 day prior to the first planned administration of serabelisib. Study ISD alone is administered. During Cycle 1 Week 1 and onward, serabelisib in combination with Study ISD and nab-paclitaxel is administered. The Study Drug is given orally on the same 3 consecutive days each week (starting with either Monday, Tuesday, or Wednesday) followed by 4 consecutive days off the Study Drug. Study Drug is administered in 28-day (4-week; +3 days) cycles without a break between cycles.

Study Schedule: For all cohorts, the study comprises, or in some cases consists of, three periods: 1) Screening and study diet Run-In (captured at Visits −1 and 0, respectively), 2) Study Drug Cycles (Visit 1 and onward), and 3) End of Intervention (to be conducted within 28 d of ending participation). An unscheduled visit should be performed in the event the subject returns to the site for a visit outside of the scheduled time points or if the subject returns for any follow-up assessments that were not captured in a regular protocol defined visit but are required for this study. The assessments during that unscheduled visit will depend on the corresponding follow-up visit but will always include safety assessments.

During the Study Drug Cycles, serabelisib, nab-paclitaxel (in Cohort 1b, and potentially in Cohorts 3 and 4) and the ISD will be administered in 28-day (4-week; ±3 d) cycles without a break between cycles. The intervention will continue until subjects develop disease progression, have unacceptable toxicity, or for other protocol-specified reasons.

In general, the procedures performed during the Screening, Run-in, Study Drug Cycles, unscheduled visits, and at the End of Intervention Visit are the same for all cohorts. Safety will be evaluated by AEs, clinical laboratory tests, vital signs, electrocardiograms (ECGs), and physical examination. Efficacy will be determined from tumor assessments (ORR, PFS, duration of response [DoR], disease control rate [DCR], and OS). Pharmacokinetic and PD samples will be used to estimate PK and PD parameters.

Screening/Run-in Period: Time Frame: 35 days (5 weeks) prior to administration of Study Drug. During the screening visit period, subjects will be evaluated for eligibility. All subjects evaluated for eligibility will be recorded on a screening log. Date of screening and reason for ineligibility (if subject is not eligible) will be recorded. No study-specific procedures that are not part of SOC should be performed prior to informed consent. Enrollment is defined as a subject who has been consented, screened, verified for eligibility, and initiated the Study ISD.

Visit −1 (Screening): Time frame: between study days −35 and −8 (between 35 and 8 days prior to run-in [Visit 0]). After obtaining informed consent, subjects will be screened for eligibility at Visit −1 (Study days −35 to −8). The screening procedures will include evidence by a fresh tumor biopsy or archival tissue confirming mutational status for PIK3CA and PTEN; if mutational status is unknown or not performed within the past 6 months, analysis of archival or fresh tumor tissue must be completed during screening.

Visit 0 (Run-In): Run-in time frame: The Run-in period must start at least 7 days before Visit 1 and thus, with Visit 0 on Day −7 or earlier provided that all other screening procedures have been successfully completed, and ends at Visit 1.

Cycles 1-4 of Study Drug Administration: For all cohorts, serabelisib will be dispensed according to the schedule of activities for administration at home in between clinic visits. Subjects will receive the serabelisib as continuous 28±3-day cycles. For Cohorts 1b, 3, and 4, nab-paclitaxel will be administered in the clinic once weekly on weeks 1, 2, and 3 of a 28 day±3 days cycle.

The Study Drug Administration Period begins at the time of serabelisib (all cohorts) and nab-paclitaxel (Cohort 1b, 3, and 4) administration at Visit 1 and will end with the completion of the End of Intervention Visit. Cohorts 1a and 1b will begin with daily 600 mg PO dosing for two weeks, and if tolerable, dosing will escalate to daily 900 mg PO for the next two weeks.

Subjects in Cohort 1b and in Cohorts 3 and 4, subsequent to evaluation of results from Cohorts 1a and 1b, will also receive nab-paclitaxel according to the current prescribing information. The starting dose level of nab-paclitaxel in this study will be 80 mg/m² administered intravenously over 30 minutes on Days 1, 8, and 15 of each 28-day cycle (3-weeks-on/1-week-off schedule, 3Q4W).

Safety assessments will be conducted throughout this dose modulation scheme to determine the RP2D to administer in Cohorts 2, 3, and 4. Subjects may remain on study (expected median of 6 months; expected maximum of 12 months) until disease progression, unacceptable toxicity, or for other protocol-specified reasons. Subjects will continue to receive the Study Drug and Study ISD until disease progression, development of unacceptable toxicity, or withdrawal of consent.

Known Potential Risks for Serabelisib: Common adverse events of serabelisib administration can include nausea, vomiting, and fatigue, and hyperglycemia. Frequently observed Grade ≥3 AEs can include elevated liver transaminases and hyperglycemia. Serabelisib has the potential to inhibit BCRP, OCT1, and OCT2. In some cases, a drug-drug interaction may be possible. The potential for BCRP inhibition can be highly unlikely particularly if the compound is highly water soluble and permeable. These medications should be administered cautiously when dosing with serabelisib, as there is the potential for serabelisib to affect the exposures of co-administered OCT1, OCT2, and BCRP substrates. If possible, these drugs should be avoided, and alternative medications prescribed. Substrates of BCRP, OCT1, and OCT2 should be administered with caution and subjects should be closely monitored. Compared with QD dosing, intermittent dosing can be associated with a lower incidence of Grade 3 drug-related ALT/AST elevations, but an increased incidence of Grade 3 drug-related hyperglycemia. In the current study, the starting dose of 600 mg PO QD is a conservative selection and was chosen because the MTD in the Phase 1 study was 900 mg on an intermittent 3CDW schedule and because there exists the potential for higher exposure of serabelisib when given with the Study ISD. In addition, the intermittent schedule given in the same 3 consecutive days out of each week (e.g., MTW, TWTh, WThF) was chosen based on the lower percentage of subjects with elevated liver enzymes in the Phase 1 study.

Known Potential Risks for Nab-Paclitaxel: Common adverse events of nab-paclitaxel include alopecia, neutropenia, sensory neuropathy, leukopenia, and non-febrile neutropenia, hyperglycemia, neutropenia, diarrhea, and rash.

Study ISD: The Study ISD formulation is consistent with other very low carbohydrate diets that have been used safely in thousands of adults and children. There are some known AEs that may occur due to the changes in water, electrolytes, and hormones. Constipation, thirst/dehydration, frequent urination, fatigue, tachycardia, lightheadedness, weight loss, and increased cholesterol can be common. People on this diet for months or years can also have decreases in some vitamins and minerals such as magnesium, calcium, vitamin D, and folate. Thus, standard QD vitamin supplements can be given. Cholelithiasis, hypoglycemia, nephrolithiasis, and hypernatremia can be possible. TABLE 25 summarizes Study ISD risk mitigation plans.

TABLE 25
Study ISD Risk Mitigation Plan

| Risk | Monitoring and Mitigation |
|---|---|
| Gastrointestinal (GI) Changes | Subjects may report any GI changes directly |
| (Constipation or Diarrhea) | to the Investigator or their research staff |
| | during study visits. Additionally, subjects |
| | may report episodes of constipation/diarrhea |
| | to the study dietitian via the App and will be |
| | transmitted to the subject's research team to |
| | provide appropriate care. Subjects for whom |
| | the exacerbation of constipation would not |
| | be advisable (e.g., small bowel obstruction |
| | hx) are excluded. |
| Dehydration | Subjects will receive recommendations for |
| | increasing fluid intake to offset risk of |
| | dehydration as applicable. |
| Fatigue | Fatigue primarily due to dehydration. |
| | Subjects will be provided education |
| | regarding increasing fluid and sodium |
| | intake. |
| Weight Loss | Meals and snacks will provide adequate |
| | calories for weight maintenance and are |
| | adjusted to provide approximately 30-35 |
| | kcal/kg body weight for subjects with BMI |
| | <30 kg/m² and 25-30 kcal/kg for subjects |
| | with BMI >30 kg/m². Subjects will enter |
| | their body weight daily. Weight is |
| | monitored on a weekly basis. Additional |
| | meals will be provided to subjects with |
| | significant weight loss, i.e., more than 10% |
| | decline compared to body weight at |
| | baseline. |
| Tachycardia | Tachycardia may occur during the transition |
| | into ketosis, commonly due to dehydration. |
| | Subjects will be provided education |
| | regarding increasing fluid and sodium |
| | intake. |
| Lightheadedness/Hypoglycemia | Subject's blood glucose and Alc will be |
| | monitored at regular intervals. Subjects with |
| | insulin-dependent diabetes are excluded. |
| | Subjects will be educated regarding the |
| | symptoms of hypoglycemia and how to |
| | treat. |

Metastatic cancer is a deadly disease and at the time subjects will be accrued to this trial, most will have a life expectancy of less than two years. In general, very low carbohydrate diets, like the Study ISD, are not only considered safe, but may be healthier than the subject's baseline diet, particularly when insulin resistance is present.

Although associated with reversible and largely tolerable side effects (e.g., GI distress and hyperglycemia), serabelisib has been generally well tolerated in most subjects with advanced cancer. The most concerning AE can be the elevation in liver transaminases observed in QD dosing, which was mitigated by using an intermittent dosing schema. The combination of serabelisib with the Study ISD is unlikely to potentiate any known side effects or elicit new ones. Diet-induced improvements in systemic metabolism may greatly diminish rates of hyperglycemia. Additionally, by using a split (300 mg TID) vs single (900 mg QD), there may be a further improvement in tolerability by decreasing nausea and vomiting which appeared to be correlated with maximal blood concentrations.

Reproductive Potential: Female subjects of child-bearing potential must have a negative serum pregnancy test at screening. They are to use an investigator-approved method of birth control from 30 days before the first administration of Study Drug to 90 days after the last administration of Study Drug. Male subjects must be surgically sterile or must agree to use physician-approved contraception during the study and for 90 days after the last administration of Study Drug.

Restrictions: During this study, subjects are asked to: Abstain from strenuous exercise for 12 hours before each blood collection for clinical laboratory tests. Arrive to each clinic visit in a fasted state (water consumption encouraged but no food for at least 8 hours prior). Refrain from all alcohol consumption. In the event of a special occasion, certain alcoholic beverages may be consumed in small quantities and subjects must correspond with the study dietitian prior to any alcohol consumption. Refrain from all outside foods beyond the Study ISD.

Study Drug (Serabelisib): Serabelisib has been formulated as immediate-release tablets for oral administration. The tablet comprises or in some cases consists of a mixture of serabelisib drug substance with microcrystalline cellulose, low substituted hydroxypropyl cellulose, croscarmellose sodium, colloidal silicon dioxide (anhydrous), and magnesium stearate. The clinical trial tablets will be supplied in 300 mg (caplet) dose strengths and administered PO.

Nab-paclitaxel: Nab-paclitaxel (ABRAXANE®) is provided as injectable suspension, for intravenous use: white to yellow, sterile lyophilized powder containing 100 mg of paclitaxel formulated as albumin-bound particles in single-dose vial for reconstitution.

### Example 6: List of Common Abbreviations

TABLE 26 shows a list of common abbreviations used herein. Any abbreviation listed may be used in its singular or plural form, depending upon the context.

TABLE 26
Common abbreviations used in this application

| Abbreviation | Definition |
|---|---|
| λz | Apparent terminal rate constant, estimated using the negative slope of the |
| | least square regression analysis of the log concentration versus time data for |
| | the terminal linear portion of the curve |
| AE | Adverse event |
| ALP | Alkaline phosphatase |
| ALT | Alanine aminotransferase |
| ANC | Absolute neutrophil count |
| AST | Aspartate aminotransferase |
| AUC | Area under the concentration versus time curve |
| AUC₀₋ₜ | Area under the concentration versus time curve from time zero to the |
| | sampling time at the last quantifiable concentration (Ct) at the time of the last |
| | quantifiable concentration (tₗₐₛₜ) calculated by the linear trapezoidal rule |
| AUC_{0-∞} | Area under the concentration versus time curve estimated from time zero to |
| | infinity as the sum of the 2 areas: AUC₀₋ₜ and AUCₑₓₜᵣₐₚ, where AUCₑₓₜᵣₐₚ is |
| | calculated as Ct/λz |
| AUCₑₓₜᵣₐₚ | Area under the plasma concentration versus time curve extrapolated from time |
| | t to infinity as a percentage of total AUC |
| BA | Bioavailability |
| BCRP | Breast cancer resistance protein |
| BHB | beta-hydroxybutyrate |
| BID | Twice daily |
| BOR | Best overall response |
| BUN | Blood urea nitrogen |
| CBC | Complete blood count |
| CFR | Code of Federal Regulations |
| CI | Confidence interval |
| CL | Systemic clearance calculated as: Dose/AUC_{0-∞} |
| C-peptide | Connecting peptide |
| Cₘₐₓ | Maximum observed plasma concentration |
| Cₘᵢₙ | Minimum observed plasma concentration |
| CR | Complete response |
| CT | Computed tomography |
| CTCAE | Common Terminology Criteria for Adverse Events |
| CYP | Cytochrome P450 |
| DLT | Dose-limiting toxicity |
| DKA | diabetic ketoacidosis |
| eCRF | Electronic Case Report Form |
| ECG | Electrocardiogram |
| ECOG PS | Eastern Cooperative Oncology Group Performance Status |
| FDA | US Food and Drug Administration |
| GCP | Good Clinical Practice |
| GI | Gastrointestinal |
| HbA1c | Hemoglobin Alc |
| HCG | Human chorionic gonadotropin |
| HIPAA | Health Insurance Portability and Accountability Act |
| HR | Hormone receptor |
| HER2 | Human epidermal growth factor receptor 2 |
| IB | Investigator's Brochure |
| IC50 | Half maximal inhibitory concentration |
| ICF | Informed Consent Form |
| ICH | International Council for Harmonization |
| IP | Investigational product |
| IRB | Institutional Review Board |
| IRT | Interactive Response Technology |
| ISD | Insulin Suppressing Diet |
| ITT | Intention-to-treat |
| LDH | Lactate dehydrogenase |
| MCH | Mean corpuscular hemoglobin |
| MCHC | Mean corpuscular hemoglobin concentration |
| MCV | Mean corpuscular volume |
| MedDRA | Medical Dictionary for Regulatory Activities |
| MRI | Magnetic resonance imaging |
| MTD | Maximum tolerated dose |
| MTW | Monday, Tuesday, and Wednesday |
| MWF | Monday, Wednesday, and Friday |
| NCI | National Cancer Institute |
| NEFA | Non-esterified fatty acids |
| NOAEL | No-observed-adverse-effect level |
| NSCLC | Non-small cell lung cancer |
| NYHA | New York Heart Association |
| OCT | Organic cation transporter |
| ORR | Objective response rate |
| OTC | Over-the-counter |
| PD | Pharmacodynamic |
| PFS | Progression-free survival |
| PI3K | Phosphoinositide 3-kinase |
| PI3Ka | Phosphoinositide 3-kinase alpha isoform |
| PI3KB | Phosphoinositide 3-kinase beta isoform |
| PIK3CA | Phosphatidylinositol-4,5-bisphosphate 3-kinase catalytic alpha |
| PK | Pharmacokinetic |
| PO | Oral |
| POC | Point of care |
| PPI | Proton pump inhibitor |
| PR | Partial response |
| PTEN | Phosphatase and tensin homolog |
| QD | Once daily |
| QTcF | QT interval corrected by Fredericia |
| RBC | Red blood cell |
| RBM | Risk-based management |
| RECIST | Response Evaluation Criteria in Solid Tumors |
| RI | Run-in |
| RP2D | Recommended phase 2 dose |
| SAE | Serious adverse event |
| SAP | Statistical Analysis Plan |
| SC | Screening |
| SD | Stable disease |
| SGLT2 | Sodium-glucose cotransporter-2 |
| SRC | Safety Review Committee |
| SUSAR | Suspected unexpected serious adverse reaction |
| t_{1/2} | Apparent terminal half-life, calculated from Ln 2/λz |
| TBD | To be determined |
| TRAE | Treatment-related adverse event |
| tₘₐₓ | Time at which Cₘₐₓ was apparent |
| ULN | Upper limit of normal |
| Vd | Apparent volume of distribution |
| Vss | Apparent volume of distribution at steady state |
| WBC | White blood cell |
| W | weeks |

### Embodiments

Embodiment 1. A method of treating a cancer in a subject in need thereof, the method comprising administering to the subject: a) an insulin suppressing meal; and b) a therapeutically effective amount of serabelisib, wherein the therapeutically effective amount is from about 300 mg to about 900 mg, thereby treating the cancer in the subject.

Embodiment 2. The method of embodiment 1, wherein the cancer comprises a PIK3Ca mutation.

Embodiment 3. The method of embodiment 1 or 2, wherein the cancer comprises a phosphatase and tensin homologue (PTEN) loss-of-function mutation.

Embodiment 4. The method of embodiment 1 or 2, wherein the cancer does not comprise a PTEN loss-of-function mutation.

Embodiment 5. The method of any one of embodiments 1-4, wherein the cancer is a solid tumor.

Embodiment 6. The method of any one of embodiments 1-5, wherein the cancer is ovarian cancer.

Embodiment 7. The method of any one of embodiments 1-5, wherein the cancer is endometrial cancer.

Embodiment 8. The method of any one of embodiments 1-5, wherein the cancer is endometrial adenocarcinoma.

Embodiment 9. The method of any one of embodiments 1-5, wherein the cancer is ovarian clear cell carcinoma.

Embodiment 10. The method of any one of embodiments 1-5, wherein the cancer is ovarian endometrioid carcinoma.

Embodiment 11. The method of any one of embodiments 1-5, wherein the cancer is colorectal cancer.

Embodiment 12. The method of any one of embodiments 1-11, wherein the subject is on an insulin suppressing diet that comprises the insulin suppressing meal.

Embodiment 13. The method of embodiment 12, wherein the insulin suppressing diet comprises at least two insulin suppressing meals daily.

Embodiment 14. The method of embodiment 12, wherein the insulin suppressing diet comprises three insulin suppressing meals daily.

Embodiment 15. The method of any one of embodiments 12-14, wherein the insulin suppressing diet comprises a net carbohydrate content of no more than about 9 g/day.

Embodiment 16. The method of any one of embodiments 12-14, wherein the insulin suppressing diet comprises a net carbohydrate content of about 0.1-9 g/day.

Embodiment 17. The method of any one of embodiments 12-14, wherein the insulin suppressing diet comprises a net carbohydrate content of about 30-40 g/day.

Embodiment 18. The method of any one of embodiments 12-17, wherein the insulin suppressing diet comprises a protein content of about 0.8-1.0 g/kg of a reference weight of the subject.

Embodiment 19. The method of any one of embodiments 12-18, wherein the insulin suppressing diet comprises a fat to protein and net carbohydrate content mass ratio of about 3:1.

Embodiment 20. The method of any one of embodiments 12-19, wherein the insulin suppressing diet comprises about 80-90% of total calories from fat.

Embodiment 21. The method of any one of embodiments 12-19, wherein the insulin suppressing diet comprises about 87% of total calories from fat.

Embodiment 22. The method of any one of embodiments 12-19, wherein the insulin suppressing diet comprises about 82% of total calories from fat.

Embodiment 23. The method of any one of embodiments 12-22, wherein the insulin suppressing diet comprises about 5-15% of total calories from protein.

Embodiment 24. The method of any one of embodiments 12-22, wherein the insulin suppressing diet comprises about 10% of total calories from protein.

Embodiment 25. The method of any one of embodiments 12-24, wherein the insulin suppressing diet comprises about 1-10% of total calories from carbohydrate.

Embodiment 26. The method of any one of embodiments 12-24, wherein the insulin suppressing diet comprises about 7% of total calories from carbohydrate.

Embodiment 27. The method of any one of embodiments 12-24, wherein the insulin suppressing diet comprises about 3% of total calories from carbohydrate.

Embodiment 28. The method of any one of embodiments 12-27, wherein the insulin suppressing diet comprises about 20-40 kcal/kg of a reference weight of the subject.

Embodiment 29. The method of any one of embodiments 12-27, wherein the insulin suppressing diet comprises about 30-35 kcal/kg of a reference weight of the subject.

Embodiment 30. The method of any one of embodiments 12-27, wherein the insulin suppressing diet comprises about 25-30 kcal/kg of a reference weight of the subject.

Embodiment 31. The method of any one of embodiments 1-30, wherein the subject has a BMI of less than about 30 kg/m².

Embodiment 32. The method of any one of embodiments 1-30, wherein the subject has a BMI of greater than about 30 kg/m².

Embodiment 33. The method of any one of embodiments 12-32, wherein the insulin suppressing diet comprises at least about 4-5 g/day of sodium.

Embodiment 34. The method of any one of embodiments 1-33, wherein the administering of the serabelisib is oral.

Embodiment 35. The method of any one of embodiments 1-34, wherein the administering of the serabelisib is once daily.

Embodiment 36. The method of any one of embodiments 1-34, wherein the administering of the serabelisib is twice daily.

Embodiment 37. The method of any one of embodiments 1-34, wherein the administering of the serabelisib is three times daily.

Embodiment 38. The method of any one of embodiments 1-37, wherein the administering of the serabelisib is three consecutive days per week.

Embodiment 39. The method of any one of embodiments 1-37, wherein the administering of the serabelisib is three consecutive days per week followed by four consecutive days without administering the serabelisib.

Embodiment 40. The method of any one of embodiments 1-37, wherein the administering of the serabelisib is three consecutive days per week followed by four consecutive days without administering the serabelisib during a 28-day cycle.

Embodiment 41. The method of any one of embodiments 1-40, wherein the administering of the serabelisib is after administering the insulin suppressing meal.

Embodiment 42. The method of any one of embodiments 1-40, wherein the administering of the serabelisib is before administering the insulin suppressing meal.

Embodiment 43. The method of any one of embodiments 1-40, wherein the administering of the serabelisib is within about 1 hour upon the subject finishing the insulin suppressing meal.

Embodiment 44. The method of any one of embodiments 1-40, wherein the administering of the serabelisib is within about 30 minutes upon the subject finishing the insulin suppressing meal.

Embodiment 45. The method of any one of embodiments 1-40, wherein the administering of the serabelisib is within about 15 minutes upon the subject finishing the insulin suppressing meal.

Embodiment 46. The method of any one of embodiments 1-45, wherein the therapeutically effective amount of serabelisib is about 300 mg.

Embodiment 47. The method of any one of embodiments 1-45, wherein the therapeutically effective amount of serabelisib is about 600 mg.

Embodiment 48. The method of any one of embodiments 1-45, wherein the therapeutically effective amount of serabelisib is about 900 mg.

Embodiment 49. The method of any one of embodiments 1-48, further comprising administering a therapeutically effective amount of a chemotherapeutic agent.

Embodiment 50. The method of embodiment 49, wherein the chemotherapeutic agent is part of a standard-of-care therapy for the cancer.

Embodiment 51. The method of embodiment 49 or 50, wherein the chemotherapeutic agent is a taxane-based chemotherapeutic agent.

Embodiment 52. The method of any one of embodiments 49-51, wherein the chemotherapeutic agent is nab-paclitaxel.

Embodiment 53. The method of any one of embodiments 49-51, wherein the chemotherapeutic agent is paclitaxel.

Embodiment 54. The method of any one of embodiments 49-51, wherein the chemotherapeutic agent is docetaxel.

Embodiment 55. The method of any one of embodiments 49-51, wherein the chemotherapeutic agent is cabazitaxel.

Embodiment 56. The method of any one of embodiments 49-55, wherein the therapeutically effective amount of the chemotherapeutic agent is about 80 mg/m².

Embodiment 57. The method of any one of embodiments 49-56, wherein the administering of the chemotherapeutic agent is over about 30 minutes.

Embodiment 58. The method of any one of embodiments 49-57, wherein the administering of the chemotherapeutic agent is once weekly.

Embodiment 59. The method of any one of embodiments 49-57, wherein the administering of the chemotherapeutic agent is once weekly for three weeks over 4 weeks (3-weeks-on/1-week-off cycle).

Embodiment 60. The method of any one of embodiments 49-59, wherein the administering of the chemotherapeutic agent is intravenous.

Embodiment 61. The method of any one of embodiments 49-59, wherein the administering of the chemotherapeutic agent is intravenous over about 30 minutes.

Embodiment 62. The method of any one of embodiments 1-61, further comprising administering a daily multivitamin, wherein the daily multivitamin comprises a daily recommended dose of essential vitamins and minerals.

Embodiment 63. The method of any one of embodiments 1-62, further comprising administering a therapeutically effective amount of an antihyperglycemia agent.

Embodiment 64. The method of embodiment 63, wherein the antihyperglycemia agent is metformin.

Embodiment 65. The method of embodiment 63, wherein the antihyperglycemia agent is acarbose.

Embodiment 66. The method of embodiment 63, wherein the antihyperglycemia agent is sodium-glucose cotransporter 2 inhibitor.

Embodiment 67. The method of embodiment 63, wherein the antihyperglycemia agent is DPP-4 inhibitor.

Embodiment 68. The method of embodiment 63, wherein the antihyperglycemia agent is a thiazolidinedione.

Embodiment 69. The method of embodiment 63, wherein the antihyperglycemia agent is a GLP-1 receptor agonist.

Embodiment 70. The method of embodiment 63, wherein the antihyperglycemia agent is an α-glucosidase inhibitor.

Embodiment 71. The method of embodiment 63, wherein the antihyperglycemia agent is a meglitinide.

Embodiment 72. The method of embodiment 63, wherein the antihyperglycemia agent is a sulfonylurea.

Embodiment 73. The method of embodiment 63, wherein the antihyperglycemia agent is insulin.

Embodiment 74. The method of any one of embodiments 49-73, further comprising administering a therapeutically effective amount of a second chemotherapeutic agent. Embodiment 75. The method of embodiment 74, wherein the second

chemotherapeutic agent is part of a standard-of-care therapy for the cancer.

Embodiment 76. The method of any one of embodiments 1-75, further comprising administering a radiotherapy.

Embodiment 77. A method of treating a cancer in a subject in need thereof, the method comprising administering to the subject: a) an insulin suppressing meal; b) a therapeutically effective amount of an inhibitor of an insulin receptor/PI3K/AKT/mTOR pathway; and c) a therapeutically effective amount of a taxane-based chemotherapeutic agent, thereby treating the cancer in the subject.

Embodiment 78. The method of embodiment 77, wherein the cancer comprises a PIK3Ca mutation.

Embodiment 79. The method of embodiment 77 or 78, wherein the cancer comprises a PTEN loss-of-function mutation.

Embodiment 80. The method of embodiment 77 or 78, wherein the cancer does not comprise a PTEN loss-of-function mutation.

Embodiment 81. The method of any one of embodiments 77-80, wherein the cancer is a solid tumor.

Embodiment 82. The method of any one of embodiments 77-81, wherein the cancer is ovarian cancer.

Embodiment 83. The method of any one of embodiments 77-81, wherein the cancer is endometrial cancer.

Embodiment 84. The method of any one of embodiments 77-81, wherein the cancer is endometrial adenocarcinoma.

Embodiment 85. The method of any one of embodiments 77-81, wherein the cancer is ovarian clear cell carcinoma.

Embodiment 86. The method of any one of embodiments 77-81, wherein the cancer is ovarian endometrioid carcinoma.

Embodiment 87. The method of any one of embodiments 77-81, wherein the cancer is colorectal cancer.

Embodiment 88. The method of any one of embodiments 77-87, wherein the subject is on an insulin suppressing diet that comprises the insulin suppressing meal.

Embodiment 89. The method of embodiment 88, wherein the insulin suppressing diet comprises at least two insulin suppressing meals daily.

Embodiment 90. The method of embodiment 88 or 89, wherein the insulin suppressing diet comprises three insulin suppressing meals daily.

Embodiment 91. The method of any one of embodiments 88-90, wherein the insulin suppressing diet comprises a net carbohydrate content of no more than about 9 g/day.

Embodiment 92. The method of any one of embodiments 88-90, wherein the insulin suppressing diet comprises a net carbohydrate content of about 0.1-9 g/day.

Embodiment 93. The method of any one of embodiments 88-90, wherein the insulin suppressing diet comprises a net carbohydrate content of about 30-40 g/day.

Embodiment 94. The method of any one of embodiments 88-93, wherein the insulin suppressing diet comprises a protein content of about 0.8-1.0 g/kg of a reference weight of the subject.

Embodiment 95. The method of any one of embodiments 88-94, wherein the insulin suppressing diet comprises a fat to protein and net carbohydrate content mass ratio of about 3:1.

Embodiment 96. The method of any one of embodiments 88-95, wherein the insulin suppressing diet comprises about 80-90% of total calories from fat.

Embodiment 97. The method of any one of embodiments 88-95, wherein the insulin suppressing diet comprises about 87% of total calories from fat.

Embodiment 98. The method of any one of embodiments 88-95, wherein the insulin suppressing diet comprises about 82% of total calories from fat.

Embodiment 99. The method of any one of embodiments 88-95, wherein the insulin suppressing diet comprises about 5-15% of total calories from protein.

Embodiment 100. The method of any one of embodiments 88-95, wherein the insulin suppressing diet comprises about 10% of total calories from protein.

Embodiment 101. The method of any one of embodiments 88-100, wherein the insulin suppressing diet comprises about 1-10% of total calories from carbohydrate.

Embodiment 102. The method of any one of embodiments 88-100, wherein the insulin suppressing diet comprises about 7% of total calories from carbohydrate.

Embodiment 103. The method of any one of embodiments 88-100, wherein the insulin suppressing diet comprises about 3% of total calories from carbohydrate.

Embodiment 104. The method of any one of embodiments 88-103, wherein the insulin suppressing diet comprises about 20-40 kcal/kg of a reference weight of the subject.

Embodiment 105. The method of any one of embodiments 88-103, wherein the insulin suppressing diet comprises about 30-35 kcal/kg of a reference weight of the subject.

Embodiment 106. The method of any one of embodiments 88-103, wherein the insulin suppressing diet comprises about 25-30 kcal/kg of a reference weight of the subject.

Embodiment 107. The method of any one of embodiments 77-106, wherein the subject has a BMI of less than about 30 kg/m².

Embodiment 108. The method of any one of embodiments 77-106, wherein the subject has a BMI of greater than about 30 kg/m².

Embodiment 109. The method of any one of embodiments 88-108, wherein the insulin suppressing diet comprises at least about 4-5 g/day of sodium.

Embodiment 110. The method of any one of embodiments 77-109, wherein the administering of the inhibitor of the insulin receptor/PI3K/AKT/mTOR pathway is oral.

Embodiment 111. The method of any one of embodiments 77-110, wherein the administering of the inhibitor of the insulin receptor/PI3K/AKT/mTOR pathway is once daily.

Embodiment 112. The method of any one of embodiments 77-110, wherein the administering of the inhibitor of the insulin receptor/PI3K/AKT/mTOR pathway is twice daily.

Embodiment 113. The method of any one of embodiments 77-110, wherein the administering of the inhibitor of the insulin receptor/PI3K/AKT/mTOR pathway is three times daily.

Embodiment 114. The method of any one of embodiments 77-113, wherein the administering of the inhibitor of the insulin receptor/PI3K/AKT/mTOR pathway is three consecutive days per week.

Embodiment 115. The method of any one of embodiments 77-113, wherein the administering of the inhibitor of the insulin receptor/PI3K/AKT/mTOR pathway is three consecutive days per week followed by four consecutive days without administering the inhibitor of the insulin receptor/PI3K/AKT/mTOR pathway.

Embodiment 116. The method of any one of embodiments 77-113, wherein the administering of the inhibitor of the insulin receptor/PI3K/AKT/mTOR pathway is three consecutive days per week followed by four consecutive days without administering the inhibitor of the insulin receptor/PI3K/AKT/mTOR pathway during a 28-day cycle.

Embodiment 117. The method of any one of embodiments 77-116, wherein the administering of the inhibitor of the insulin receptor/PI3K/AKT/mTOR pathway is after administering the insulin suppressing meal.

Embodiment 118. The method of any one of embodiments 77-116, wherein the administering of the inhibitor of the insulin receptor/PI3K/AKT/mTOR pathway is before administering the insulin suppressing meal.

Embodiment 119. The method of any one of embodiments 77-116, wherein the administering of the inhibitor of the insulin receptor/PI3K/AKT/mTOR pathway is within about 1 hour upon the subject finishing the insulin suppressing meal.

Embodiment 120. The method of any one of embodiments 77-116, wherein the administering of the inhibitor of the insulin receptor/PI3K/AKT/mTOR pathway is within about 30 minutes upon the subject finishing the insulin suppressing meal.

Embodiment 121. The method of any one of embodiments 77-116, wherein the administering of the inhibitor of the insulin receptor/PI3K/AKT/mTOR pathway is within about 15 minutes upon the subject finishing the insulin suppressing meal.

Embodiment 122. The method of any one of embodiments 77-121, wherein the therapeutically effective amount of inhibitor of the insulin receptor/PI3K/AKT/mTOR pathway is about 300 mg.

Embodiment 123. The method of any one of embodiments 77-121, wherein the therapeutically effective amount of inhibitor of the insulin receptor/PI3K/AKT/mTOR pathway is about 600 mg.

Embodiment 124. The method of any one of embodiments 77-121, wherein the therapeutically effective amount of inhibitor of the insulin receptor/PI3K/AKT/mTOR pathway is about 900 mg.

Embodiment 125. The method of any one of embodiments 77-124, wherein the inhibitor of the insulin receptor/PI3K/AKT/mTOR pathway is a PI3K inhibitor.

Embodiment 126. The method of any one of embodiments 77-125, wherein the inhibitor of the insulin receptor/PI3K/AKT/mTOR pathway is a PI3Kα inhibitor.

Embodiment 127. The method of any one of embodiments 77-125, wherein the inhibitor of the insulin receptor/PI3K/AKT/mTOR pathway is serabelisib.

Embodiment 128. The method of any one of embodiments 77-125, wherein the inhibitor of the insulin receptor/PI3K/AKT/mTOR pathway is idelalisib.

Embodiment 129. The method of any one of embodiments 77-125, wherein the inhibitor of the insulin receptor/PI3K/AKT/mTOR pathway is copanlisib.

Embodiment 130. The method of any one of embodiments 77-125, wherein the inhibitor of the insulin receptor/PI3K/AKT/mTOR pathway is buparlisib (BKM120).

Embodiment 131. The method of any one of embodiments 77-125, wherein the inhibitor of the insulin receptor/PI3K/AKT/mTOR pathway is alpelisib (BYL719).

Embodiment 132. The method of any one of embodiments 77-125, wherein the inhibitor of the insulin receptor/PI3K/AKT/mTOR pathway is taselisib (GDC-0032).

Embodiment 133. The method of any one of embodiments 77-125, wherein the inhibitor of the insulin receptor/PI3K/AKT/mTOR pathway is pictilisib (GDC-0941).

Embodiment 134. The method of any one of embodiments 77-125, wherein the inhibitor of the insulin receptor/PI3K/AKT/mTOR pathway is apitolisib (GDC-0980).

Embodiment 135. The method of any one of embodiments 77-125, wherein the inhibitor of the insulin receptor/PI3K/AKT/mTOR pathway is dactolisib.

Embodiment 136. The method of any one of embodiments 77-124, wherein the inhibitor of the insulin receptor/PI3K/AKT/mTOR pathway is an AKT inhibitor.

Embodiment 137. The method of any one of embodiments 77-124 and 136, wherein the inhibitor of the insulin receptor/PI3K/AKT/mTOR pathway is MK-2206.

Embodiment 138. The method of any one of embodiments 77-124, wherein the inhibitor of the insulin receptor/PI3K/AKT/mTOR pathway is an inhibitor of the insulin receptor.

Embodiment 139. The method of any one of embodiments 77-124 and 138, wherein the inhibitor of the insulin receptor/PI3K/AKT/mTOR pathway is linsitinib.

Embodiment 140. The method of any one of embodiments 77-139, wherein the taxane-based chemotherapeutic agent is nab-paclitaxel.

Embodiment 141. The method of any one of embodiments 77-139, wherein the taxane-based chemotherapeutic agent is paclitaxel.

Embodiment 142. The method of any one of embodiments 77-139, wherein the taxane-based chemotherapeutic agent is docetaxel.

Embodiment 143. The method of any one of embodiments 77-139, wherein the taxane-based chemotherapeutic agent is cabazitaxel.

Embodiment 144. The method of any one of embodiments 77-143, wherein the therapeutically effective amount of the taxane-based chemotherapeutic agent is about 80 mg/m².

Embodiment 145. The method of any one of embodiments 77-144, wherein the administering of the taxane-based chemotherapeutic agent is over about 30 minutes.

Embodiment 146. The method of any one of embodiments 77-145, wherein the administering of the taxane-based chemotherapeutic agent is once weekly.

Embodiment 147. The method of any one of embodiments 77-145, wherein the administering of the taxane-based chemotherapeutic agent is once weekly for three weeks over 4 weeks (3-weeks-on/1-week-off cycle).

Embodiment 148. The method of any one of embodiments 77-147, wherein the administering of the taxane-based chemotherapeutic agent is intravenous.

Embodiment 149. The method of any one of embodiments 77-147, wherein the administering of the taxane-based chemotherapeutic agent is intravenous over about 30 minutes.

Embodiment 150. The method of any one of embodiments 77-149, further comprising administering a daily multivitamin, wherein the daily multivitamin comprises a daily recommended dose of essential vitamins and minerals.

Embodiment 151. The method of any one of embodiments 77-150, further comprising administering a therapeutically effective amount of an antihyperglycemia agent.

Embodiment 152. The method of embodiment 151, wherein the antihyperglycemia agent is metformin.

Embodiment 153. The method of embodiment 151, wherein the antihyperglycemia agent is acarbose.

Embodiment 154. The method of embodiment 151, wherein the antihyperglycemia agent is sodium-glucose cotransporter 2 inhibitor.

Embodiment 155. The method of embodiment 151, wherein the antihyperglycemia agent is DPP-4 inhibitor.

Embodiment 156. The method of embodiment 151, wherein the antihyperglycemia agent is a thiazolidinedione.

Embodiment 157. The method of embodiment 151, wherein the antihyperglycemia agent is a GLP-1 receptor agonist.

Embodiment 158. The method of embodiment 151, wherein the antihyperglycemia agent is an α-glucosidase inhibitor.

Embodiment 159. The method of embodiment 151, wherein the antihyperglycemia agent is a meglitinide.

Embodiment 160. The method of embodiment 151, wherein the antihyperglycemia agent is a sulfonylurea.

Embodiment 161. The method of embodiment 151, wherein the antihyperglycemia agent is insulin.

Embodiment 162. The method of any one of embodiments 77-161, further comprising administering a therapeutically effective amount of a second chemotherapeutic agent.

Embodiment 163. The method of embodiment 162, wherein the second chemotherapeutic agent is part of a standard-of-care therapy for the cancer.

Embodiment 164. The method of any one of embodiments 77-163, further comprising administering a radiotherapy.

Embodiment 165. A method of treating a cancer in a subject in need thereof, the method comprising administering to the subject: a) an insulin suppressing meal; and b) a therapeutically effective amount of serabelisib, wherein the therapeutically effective amount is from about 300 mg to about 900 mg daily for three consecutive days per week, wherein the administration of the serabelisib is oral, wherein the cancer is a solid tumor comprising a PIK3CA mutation, thereby treating the cancer in the subject.

Embodiment 166. The method of embodiment 165, wherein the cancer comprises a PTEN loss-of-function mutation.

Embodiment 167. The method of embodiment 165, wherein the cancer does not comprise a PTEN loss-of-function mutation.

Embodiment 168. The method of any one of embodiments 165-167, wherein the cancer is ovarian cancer.

Embodiment 169. The method of any one of embodiments 165-167, wherein the cancer is endometrial cancer.

Embodiment 170. The method of any one of embodiments 165-167, wherein the cancer is endometrial adenocarcinoma.

Embodiment 171. The method of any one of embodiments 165-167, wherein the cancer is ovarian clear cell carcinoma.

Embodiment 172. The method of any one of embodiments 165-167, wherein the cancer is ovarian endometrioid carcinoma.

Embodiment 173. The method of any one of embodiments 165-167, wherein the cancer is colorectal cancer.

Embodiment 174. The method of any one of embodiments 165-173, wherein the subject is on an insulin suppressing diet that comprises the insulin suppressing meal.

Embodiment 175. The method of embodiment 174, wherein the insulin suppressing diet comprises at least two insulin suppressing meals daily.

Embodiment 176. The method of embodiment 174 or 175, wherein the insulin suppressing diet comprises three insulin suppressing meals daily.

Embodiment 177. The method of any one of embodiments 174-176, wherein the insulin suppressing diet comprises a net carbohydrate content of no more than about 9 g/day.

Embodiment 178. The method of any one of embodiments 174-176, wherein the insulin suppressing diet comprises a net carbohydrate content of about 0.1-9 g/day.

Embodiment 179. The method of any one of embodiments 174-176, wherein the insulin suppressing diet comprises a net carbohydrate content of about 30-40 g/day.

Embodiment 180. The method of any one of embodiments 174-179, wherein the insulin suppressing diet comprises a protein content of about 0.8-1.0 g/kg of a reference weight of the subject.

Embodiment 181. The method of any one of embodiments 174-178, wherein the insulin suppressing diet comprises a fat to protein and net carbohydrate content mass ratio of about 3:1.

Embodiment 182. The method of any one of embodiments 174-181, wherein the insulin suppressing diet comprises about 80-90% of total calories from fat.

Embodiment 183. The method of any one of embodiments 174-181, wherein the insulin suppressing diet comprises about 87% of total calories from fat.

Embodiment 184. The method of any one of embodiments 174-181, wherein the insulin suppressing diet comprises about 82% of total calories from fat.

Embodiment 185. The method of any one of embodiments 174-184, wherein the insulin suppressing diet comprises about 5-15% of total calories from protein.

Embodiment 186. The method of any one of embodiments 174-184, wherein the insulin suppressing diet comprises about 10% of total calories from protein.

Embodiment 187. The method of any one of embodiments 174-186, wherein the insulin suppressing diet comprises about 1-10% of total calories from carbohydrate.

Embodiment 188. The method of any one of embodiments 174-186, wherein the insulin suppressing diet comprises about 7% of total calories from carbohydrate.

Embodiment 189. The method of any one of embodiments 174-186, wherein the insulin suppressing diet comprises about 3% of total calories from carbohydrate.

Embodiment 190. The method of any one of embodiments 174-189, wherein the insulin suppressing diet comprises about 20-40 kcal/kg of a reference weight of the subject.

Embodiment 191. The method of any one of embodiments 174-189, wherein the insulin suppressing diet comprises about 30-35 kcal/kg of a reference weight of the subject.

Embodiment 192. The method of any one of embodiments 174-189, wherein the insulin suppressing diet comprises about 25-30 kcal/kg of a reference weight of the subject.

Embodiment 193. The method of any one of embodiments 165-192, wherein the subject has a BMI of less than about 30 kg/m².

Embodiment 194. The method of any one of embodiments 165-192, wherein the subject has a BMI of greater than about 30 kg/m².

Embodiment 195. The method of any one of embodiments 174-194, wherein the insulin suppressing diet comprises at least about 4-5 g/day of sodium.

Embodiment 196. The method of any one of embodiments 165-195, wherein the administering of the serabelisib is twice daily.

Embodiment 197. The method of any one of embodiments 165-195, wherein the administering of the serabelisib is three times daily.

Embodiment 198. The method of any one of embodiments 165-197, wherein the administering of the serabelisib is three consecutive days per week followed by four consecutive days without administering the serabelisib.

Embodiment 199. The method of any one of embodiments 165-197, wherein the administering of the serabelisib is three consecutive days per week followed by four consecutive days without administering the serabelisib during a 28-day cycle.

Embodiment 200. The method of any one of embodiments 165-199, wherein the administering of the serabelisib is after administering the insulin suppressing meal.

Embodiment 201. The method of any one of embodiments 165-199, wherein the administering of the serabelisib is before administering the insulin suppressing meal.

Embodiment 202. The method of any one of embodiments 165-199, wherein the administering of the serabelisib is within about 1 hour upon the subject finishing the insulin suppressing meal.

Embodiment 203. The method of any one of embodiments 165-199, wherein the administering of the serabelisib is within about 30 minutes upon the subject finishing the insulin suppressing meal.

Embodiment 204. The method of any one of embodiments 165-199, wherein the administering of the serabelisib is within about 15 minutes upon the subject finishing the insulin suppressing meal.

Embodiment 205. The method of any one of embodiments 165-204, wherein the therapeutically effective amount of serabelisib is about 300 mg.

Embodiment 206. The method of any one of embodiments 165-204, wherein the therapeutically effective amount of serabelisib is about 600 mg.

Embodiment 207. The method of any one of embodiments 165-204, wherein the therapeutically effective amount of serabelisib is about 900 mg.

Embodiment 208. The method of any one of embodiments 165-207, further comprising administering a therapeutically effective amount of a chemotherapeutic agent.

Embodiment 209. The method of embodiment 208, wherein the chemotherapeutic agent is part of a standard-of-care therapy for the cancer.

Embodiment 210. The method of embodiment 208 or 209, wherein the chemotherapeutic agent is a taxane-based chemotherapeutic agent.

Embodiment 211. The method of any one of embodiments 208-210, wherein the chemotherapeutic agent is nab-paclitaxel.

Embodiment 212. The method of any one of embodiments 208-210, wherein the chemotherapeutic agent is paclitaxel.

Embodiment 213. The method of any one of embodiments 208-210, wherein the chemotherapeutic agent is docetaxel.

Embodiment 214. The method of any one of embodiments 208-210, wherein the chemotherapeutic agent is cabazitaxel.

Embodiment 215. The method of any one of embodiments 208-214, wherein the therapeutically effective amount of the chemotherapeutic agent is about 80 mg/m².

Embodiment 216. The method of any one of embodiments 208-215, wherein the administering of the chemotherapeutic agent is over about 30 minutes.

Embodiment 217. The method of any one of embodiments 208-216, wherein the administering of the chemotherapeutic agent is once weekly.

Embodiment 218. The method of any one of embodiments 208-216, wherein the administering of the chemotherapeutic agent is once weekly for three weeks over 4 weeks (3-weeks-on/1-week-off cycle).

Embodiment 219. The method of any one of embodiments 208-218, wherein the administering of the chemotherapeutic agent is intravenous.

Embodiment 220. The method of any one of embodiments 208-218, wherein the administering of the chemotherapeutic agent is intravenous over about 30 minutes.

Embodiment 221. The method of any one of embodiments 165-220, further comprising administering a daily multivitamin, wherein the daily multivitamin comprises a daily recommended dose of essential vitamins and minerals.

Embodiment 222. The method of any one of embodiments 165-221, further comprising administering a therapeutically effective amount of an antihyperglycemia agent.

Embodiment 223. The method of embodiment 222, wherein the antihyperglycemia agent is metformin.

Embodiment 224. The method of embodiment 222, wherein the antihyperglycemia agent is acarbose.

Embodiment 225. The method of embodiment 222, wherein the antihyperglycemia agent is sodium-glucose cotransporter 2 inhibitor.

Embodiment 226. The method of embodiment 222, wherein the antihyperglycemia agent is DPP-4 inhibitor.

Embodiment 227. The method of embodiment 222, wherein the antihyperglycemia agent is a thiazolidinedione.

Embodiment 228. The method of embodiment 222, wherein the antihyperglycemia agent is a GLP-1 receptor agonist.

Embodiment 229. The method of embodiment 222, wherein the antihyperglycemia agent is an α-glucosidase inhibitor.

Embodiment 230. The method of embodiment 222, wherein the antihyperglycemia agent is a meglitinide.

Embodiment 231. The method of embodiment 222, wherein the antihyperglycemia agent is a sulfonylurea.

Embodiment 232. The method of embodiment 222, wherein the antihyperglycemia agent is insulin.

Embodiment 233. The method of any one of embodiments 165-232, further comprising administering a therapeutically effective amount of a second chemotherapeutic agent.

Embodiment 234. The method of embodiment 233, wherein the second chemotherapeutic agent is part of a standard-of-care therapy for the cancer.

Embodiment 235. The method of any one of embodiments 165-234, further comprising administering a radiotherapy.

Embodiment 236. A method of treating a cancer in a subject in need thereof, the method comprising administering to the subject: a) an insulin suppressing meal; b) a therapeutically effective amount of serabelisib, wherein the therapeutically effective amount is from about 300 mg to about 900 mg; and c) a therapeutically effective amount of nab-paclitaxel, wherein the therapeutically effective amount is about 80 mg/m², wherein the administration of the serabelisib is oral, wherein the administering of the serabelisib is daily for three consecutive days per week, wherein the administration of the nab-paclitaxel is intravenous, wherein the administration of the nab-paclitaxel is once weekly, thereby treating the cancer in the subject.

Embodiment 237. The method of embodiment 236, wherein the cancer comprises a PIK3Ca mutation.

Embodiment 238. The method of embodiment 236 or 237, wherein the cancer comprises a PTEN loss-of-function mutation.

Embodiment 239. The method of embodiment 236 or 237, wherein the cancer does not comprise a PTEN loss-of-function mutation.

Embodiment 240. The method of any one of embodiments 236-239, wherein the cancer is a solid tumor.

Embodiment 241. The method of any one of embodiments 236-240, wherein the cancer is ovarian cancer.

Embodiment 242. The method of any one of embodiments 236-240, wherein the cancer is endometrial cancer.

Embodiment 243. The method of any one of embodiments 236-240, wherein the cancer is endometrial adenocarcinoma.

Embodiment 244. The method of any one of embodiments 236-240, wherein the cancer is ovarian clear cell carcinoma.

Embodiment 245. The method of any one of embodiments 236-240, wherein the cancer is ovarian endometrioid carcinoma.

Embodiment 246. The method of any one of embodiments 236-240, wherein the cancer is colorectal cancer.

Embodiment 247. The method of any one of embodiments 236-246, wherein the subject is on an insulin suppressing diet that comprises the insulin suppressing meal.

Embodiment 248. The method of embodiment 247, wherein the insulin suppressing diet comprises at least two insulin suppressing meals daily.

Embodiment 249. The method of embodiment 247 or 248, wherein the insulin suppressing diet comprises three insulin suppressing meals daily.

Embodiment 250. The method of any one of embodiments 247-249, wherein the insulin suppressing diet comprises a net carbohydrate content of no more than about 9 g/day.

Embodiment 251. The method of any one of embodiments 247-249, wherein the insulin suppressing diet comprises a net carbohydrate content of about 0.1-9 g/day.

Embodiment 252. The method of any one of embodiments 247-249, wherein the insulin suppressing diet comprises a net carbohydrate content of about 30-40 g/day.

Embodiment 253. The method of any one of embodiments 247-252, wherein the insulin suppressing diet comprises a protein content of about 0.8-1.0 g/kg of a reference weight of the subject.

Embodiment 254. The method of any one of embodiments 247-253, wherein the insulin suppressing diet comprises a fat to protein and net carbohydrate content mass ratio of about 3:1.

Embodiment 255. The method of any one of embodiments 247-254, wherein the insulin suppressing diet comprises about 80-90% of total calories from fat.

Embodiment 256. The method of any one of embodiments 247-254, wherein the insulin suppressing diet comprises about 87% of total calories from fat.

Embodiment 257. The method of any one of embodiments 247-254, wherein the insulin suppressing diet comprises about 82% of total calories from fat.

Embodiment 258. The method of any one of embodiments 247-257, wherein the insulin suppressing diet comprises about 5-15% of total calories from protein.

Embodiment 259. The method of any one of embodiments 247-257, wherein the insulin suppressing diet comprises about 10% of total calories from protein.

Embodiment 260. The method of any one of embodiments 247-259, wherein the insulin suppressing diet comprises about 1-10% of total calories from carbohydrate.

Embodiment 261. The method of any one of embodiments 247-259, wherein the insulin suppressing diet comprises about 7% of total calories from carbohydrate.

Embodiment 262. The method of any one of embodiments 247-259, wherein the insulin suppressing diet comprises about 3% of total calories from carbohydrate.

Embodiment 263. The method of any one of embodiments 247-262, wherein the insulin suppressing diet comprises about 20-40 kcal/kg of a reference weight of the subject.

Embodiment 264. The method of any one of embodiments 247-262, wherein the insulin suppressing diet comprises about 30-35 kcal/kg of a reference weight of the subject.

Embodiment 265. The method of any one of embodiments 247-262, wherein the insulin suppressing diet comprises about 25-30 kcal/kg of a reference weight of the subject.

Embodiment 266. The method of any one of embodiments 236-265, wherein the subject has a BMI of less than about 30 kg/m².

Embodiment 267. The method of any one of embodiments 236-265, wherein the subject has a BMI of greater than about 30 kg/m².

Embodiment 268. The method of any one of embodiments 247-267, wherein the insulin suppressing diet comprises at least about 4-5 g/day of sodium.

Embodiment 269. The method of any one of embodiments 236-268, wherein the administering of the serabelisib is once daily.

Embodiment 270. The method of any one of embodiments 236-268, wherein the administering of the serabelisib is twice daily.

Embodiment 271. The method of any one of embodiments 236-268, wherein the administering of the serabelisib is three times daily.

Embodiment 272. The method of any one of embodiments 236-271, wherein the administering of the serabelisib is three consecutive days per week followed by four consecutive days without administering the serabelisib.

Embodiment 273. The method of any one of embodiments 236-271, wherein the administering of the serabelisib is three consecutive days per week followed by four consecutive days without administering the serabelisib during a 28-day cycle.

Embodiment 274. The method of any one of embodiments 236-273, wherein the administering of the serabelisib is after administering the insulin suppressing meal.

Embodiment 275. The method of any one of embodiments 236-273, wherein the administering of the serabelisib is before administering the insulin suppressing meal.

Embodiment 276. The method of any one of embodiments 236-273, wherein the administering of the serabelisib is within about 1 hour upon the subject finishing the insulin suppressing meal.

Embodiment 277. The method of any one of embodiments 236-273, wherein the administering of the serabelisib is within about 30 minutes upon the subject finishing the insulin suppressing meal.

Embodiment 278. The method of any one of embodiments 236-273, wherein the administering of the serabelisib is within about 15 minutes upon the subject finishing the insulin suppressing meal.

Embodiment 279. The method of any one of embodiments 236-278, wherein the therapeutically effective amount of serabelisib is about 300 mg.

Embodiment 280. The method of any one of embodiments 236-278, wherein the therapeutically effective amount of serabelisib is about 600 mg.

Embodiment 281. The method of any one of embodiments 236-278, wherein the therapeutically effective amount of serabelisib is about 900 mg.

Embodiment 282. The method of any one of embodiments 236-281, wherein the administering of nab-paclitaxel is over about 30 minutes.

Embodiment 283. The method of any one of embodiments 236-282, wherein the administering of the nab-paclitaxel is once weekly for three weeks over 4 weeks (3-weeks-on/1-week-off cycle).

Embodiment 284. The method of any one of embodiments 236-283, further comprising administering a daily multivitamin, wherein the daily multivitamin comprises a daily recommended dose of essential vitamins and minerals.

Embodiment 285. The method of any one of embodiments 236-284, further comprising administering a therapeutically effective amount of an antihyperglycemia agent.

Embodiment 286. The method of embodiment 285, wherein the antihyperglycemia agent is metformin.

Embodiment 287. The method of embodiment 285, wherein the antihyperglycemia agent is acarbose.

Embodiment 288. The method of embodiment 285, wherein the antihyperglycemia agent is sodium-glucose cotransporter 2 inhibitor.

Embodiment 289. The method of embodiment 285, wherein the antihyperglycemia agent is DPP-4 inhibitor.

Embodiment 290. The method of embodiment 285, wherein the antihyperglycemia agent is a thiazolidinedione.

Embodiment 291. The method of embodiment 285, wherein the antihyperglycemia agent is a GLP-1 receptor agonist.

Embodiment 292. The method of embodiment 285, wherein the antihyperglycemia agent is an α-glucosidase inhibitor.

Embodiment 293. The method of embodiment 285, wherein the antihyperglycemia agent is a meglitinide.

Embodiment 294. The method of embodiment 285, wherein the antihyperglycemia agent is a sulfonylurea.

Embodiment 295. The method of embodiment 285, wherein the antihyperglycemia agent is insulin.

Embodiment 296. The method of any one of embodiments 236-295 further comprising administering a therapeutically effective amount of a second chemotherapeutic agent.

Embodiment 297. The method of embodiment 296, wherein the second chemotherapeutic agent is part of a standard-of-care therapy for the cancer.

Embodiment 298. The method of any one of embodiments 236-297, further comprising administering a radiotherapy.

Embodiment 299. A method of treating a cancer in a subject in need thereof, the method comprising administering to the subject: a) an insulin suppressing meal; and b) a therapeutically effective amount of a PI3K inhibitor, thereby treating the cancer in the subject.

Embodiment 300. A method of inhibiting PI3K signaling in a subject in need thereof, the method comprising administering to the subject: a) an insulin suppressing meal; and b) a therapeutically effective amount of a PI3K inhibitor, thereby inhibiting PI3K signaling in the subject.

Embodiment 301. The method of embodiment 299, wherein the cancer comprises a PIK3Ca mutation.

Embodiment 302. The method of embodiment 299 or 301, wherein the cancer comprises a PTEN loss-of-function mutation.

Embodiment 303. The method of embodiment 299 or 301, wherein the cancer does not comprise a PTEN loss-of-function mutation.

Embodiment 304. The method of any one of embodiments 299 and 301-303, wherein the cancer is a solid tumor.

Embodiment 305. The method of any one of embodiments 299 and 301-304, wherein the cancer is ovarian cancer.

Embodiment 306. The method of any one of embodiments 299 and 301-304, wherein the cancer is endometrial cancer.

Embodiment 307. The method of any one of embodiments 299 and 301-304, wherein the cancer is endometrial adenocarcinoma.

Embodiment 308. The method of any one of embodiments 299 and 301-304, wherein

the cancer is ovarian clear cell carcinoma.

Embodiment 309. The method of any one of embodiments 299 and 301-304, wherein

the cancer is ovarian endometrioid carcinoma.

Embodiment 310. The method of any one of embodiments 299 and 301-304, wherein the cancer is colorectal cancer.

Embodiment 311. The method of embodiment 300, wherein the subject has a cancer.

Embodiment 312. The method of embodiment 311, wherein the cancer comprises a PIK3Ca mutation.

Embodiment 313. The method of any one of embodiments 300, 311, and 312, wherein the cancer comprises a PTEN loss-of-function mutation.

Embodiment 314. The method of any one of embodiments 300, 311, and 312, wherein the cancer does not comprise a PTEN loss-of-function mutation.

Embodiment 315. The method of any one of embodiments 311-314, wherein the cancer is a solid tumor.

Embodiment 316. The method of any one of embodiments 311-315, wherein the cancer is ovarian cancer.

Embodiment 317. The method of any one of embodiments 311-315, wherein the cancer is endometrial cancer.

Embodiment 318. The method of any one of embodiments 311-315, wherein the cancer is endometrial adenocarcinoma.

Embodiment 319. The method of any one of embodiments 311-315, wherein the cancer is ovarian clear cell carcinoma.

Embodiment 320. The method of any one of embodiments 311-315, wherein the cancer is ovarian endometrioid carcinoma.

Embodiment 321. The method of any one of embodiments 311-315, wherein the cancer is colorectal cancer.

Embodiment 322. The method of any one of embodiments 299-321, wherein the subject is on an insulin suppressing diet that comprises the insulin suppressing meal.

Embodiment 323. The method of embodiment 322, wherein the insulin suppressing diet comprises at least two insulin suppressing meals daily.

Embodiment 324. The method of embodiment 322 or 323, wherein the insulin suppressing diet comprises three insulin suppressing meals daily.

Embodiment 325. The method of any one of embodiments 322-324, wherein the insulin suppressing diet comprises a net carbohydrate content of no more than about 9 g/day.

Embodiment 326. The method of any one of embodiments 322-324, wherein the insulin suppressing diet comprises a net carbohydrate content of about 0.1-9 g/day.

Embodiment 327. The method of any one of embodiments 322-324, wherein the insulin suppressing diet comprises a net carbohydrate content of about 30-40 g/day.

Embodiment 328. The method of any one of embodiments 322-327, wherein the insulin suppressing diet comprises a protein content of about 0.8-1.0 g/kg of a reference weight of the subject.

Embodiment 329. The method of any one of embodiments 322-328, wherein the insulin suppressing diet comprises a fat to protein and net carbohydrate content mass ratio of about 3:1.

Embodiment 330. The method of any one of embodiments 322-329, wherein the insulin suppressing diet comprises about 80-90% of total calories from fat.

Embodiment 331. The method of any one of embodiments 322-329, wherein the insulin suppressing diet comprises about 87% of total calories from fat.

Embodiment 332. The method of any one of embodiments 322-329, wherein the insulin suppressing diet comprises about 82% of total calories from fat.

Embodiment 333. The method of any one of embodiments 322-332, wherein the insulin suppressing diet comprises about 5-15% of total calories from protein.

Embodiment 334. The method of any one of embodiments 322-332, wherein the insulin suppressing diet comprises about 10% of total calories from protein.

Embodiment 335. The method of any one of embodiments 322-334, wherein the insulin suppressing diet comprises about 1-10% of total calories from carbohydrate.

T Embodiment 336. The method of any one of embodiments 322-334, wherein the insulin suppressing diet comprises about 7% of total calories from carbohydrate.

Embodiment 337. The method of any one of embodiments 322-334, wherein the insulin suppressing diet comprises about 3% of total calories from carbohydrate.

Embodiment 338. The method of any one of embodiments 322-337, wherein the insulin suppressing diet comprises about 20-40 kcal/kg of a reference weight of the subject.

Embodiment 339. The method of any one of embodiments 322-337, wherein the insulin suppressing diet comprises about 30-35 kcal/kg of a reference weight of the subject.

Embodiment 340. The method of any one of embodiments 322-337, wherein the insulin suppressing diet comprises about 25-30 kcal/kg of a reference weight of the subject.

Embodiment 341. The method of any one of embodiments 299-340, wherein the subject has a BMI of less than about 30 kg/m².

Embodiment 342. The method of any one of embodiments 299-340, wherein the subject has a BMI of greater than about 30 kg/m².

Embodiment 343. The method of any one of embodiments 322-342, wherein the insulin suppressing diet comprises at least about 4-5 g/day of sodium.

Embodiment 344. The method of any one of embodiments 299-343, wherein the administering of the PI3K inhibitor is oral.

Embodiment 345. The method of any one of embodiments 299-344, wherein the administering of the PI3K inhibitor is once daily.

Embodiment 346. The method of any one of embodiments 299-344, wherein the administering of the PI3K inhibitor is twice daily.

Embodiment 347. The method of any one of embodiments 299-344, wherein the administering of the PI3K inhibitor is three times daily.

Embodiment 348. The method of any one of embodiments 299-347, wherein the administering of the PI3K inhibitor is three consecutive days per week.

Embodiment 349. The method of any one of embodiments 299-347, wherein the administering of the PI3K inhibitor is three consecutive days per week followed by four consecutive days without administering of the PI3K inhibitor.

Embodiment 350. The method of any one of embodiments 299-347, wherein the administering of the PI3K inhibitor is three consecutive days per week followed by four consecutive days without administering of the PI3K inhibitor during a 28-day cycle.

Embodiment 351. The method of any one of embodiments 299-350, wherein the administering of the PI3K inhibitor is after administering the insulin suppressing meal.

Embodiment 352. The method of any one of embodiments 299-350, wherein the administering of the PI3K inhibitor is before administering the insulin suppressing meal.

Embodiment 353. The method of any one of embodiments 299-350, wherein the administering of the PI3K inhibitor is within about 1 hour upon the subject finishing the insulin suppressing meal.

Embodiment 354. The method of any one of embodiments 299-350, wherein the administering of the PI3K inhibitor is within about 30 minutes upon the subject finishing the insulin suppressing meal.

Embodiment 355. The method of any one of embodiments 299-350, wherein the administering of the PI3K inhibitor is within about 15 minutes upon the subject finishing the insulin suppressing meal.

Embodiment 356. The method of any one of embodiments 299-355, wherein the therapeutically effective amount of the PI3K inhibitor is about 300 mg.

Embodiment 357. The method of any one of embodiments 299-355, wherein the therapeutically effective amount of the PI3K inhibitor is about 600 mg.

Embodiment 358. The method of any one of embodiments 299-355, wherein the therapeutically effective amount of the PI3K inhibitor is about 900 mg.

Embodiment 359. The method of any one of embodiments 299-358, wherein the PI3K inhibitor is a PI3Kα inhibitor.

Embodiment 360. The method of any one of embodiments 299-358, wherein the PI3K inhibitor is serabelisib.

Embodiment 361. The method of any one of embodiments 299-358, wherein the PI3K inhibitor is idelalisib.

Embodiment 362. The method of any one of embodiments 299-358, wherein the PI3K inhibitor is copanlisib.

Embodiment 363. The method of any one of embodiments 299-358, wherein the PI3K inhibitor is buparlisib (BKM120).

Embodiment 364. The method of any one of embodiments 299-358, wherein the PI3K inhibitor is alpelisib (BYL719).

Embodiment 365. The method of any one of embodiments 299-358, wherein the PI3K inhibitor is taselisib (GDC-0032).

Embodiment 366. The method of any one of embodiments 299-358, wherein the PI3K inhibitor is pictilisib (GDC-0941).

Embodiment 367. The method of any one of embodiments 299-358, wherein the PI3K inhibitor is apitolisib (GDC-0980).

Embodiment 368. The method of any one of embodiments 299-358, wherein the PI3K inhibitor is dactolisib.

Embodiment 369. The method of any one of embodiments 299-368, further comprising administering a therapeutically effective amount of a chemotherapeutic agent.

Embodiment 370. The method of embodiment 369, wherein the chemotherapeutic agent is part of a standard-of-care therapy for the cancer.

Embodiment 371. The method of embodiment 369 or 370, wherein the chemotherapeutic agent is a taxane-based chemotherapeutic agent.

Embodiment 372. The method of any one of embodiments 369-371, wherein the chemotherapeutic agent is nab-paclitaxel.

Embodiment 373. The method of any one of embodiments 369-371, wherein the chemotherapeutic agent is paclitaxel.

Embodiment 374. The method of any one of embodiments 369-371, wherein the chemotherapeutic agent is docetaxel.

Embodiment 375. The method of any one of embodiments 369-371, wherein the chemotherapeutic agent is cabazitaxel.

Embodiment 376. The method of any one of embodiments 369-375, wherein the therapeutically effective amount of the chemotherapeutic agent is about 80 mg/m².

Embodiment 377. The method of any one of embodiments 369-376, wherein the administering of the chemotherapeutic agent is over about 30 minutes.

Embodiment 378. The method of any one of embodiments 369-377, wherein the administering of the chemotherapeutic agent is once weekly.

Embodiment 379. The method of any one of embodiments 369-377, wherein the administering of the chemotherapeutic agent is once weekly for three weeks over 4 weeks (3-weeks-on/1-week-off cycle).

Embodiment 380. The method of any one of embodiments 369-379, wherein the administering of the chemotherapeutic agent is intravenous.

Embodiment 381. The method of any one of embodiments 369-379, wherein the administering of the chemotherapeutic agent is intravenous over about 30 minutes.

Embodiment 382. The method of any one of embodiments 299-381, further comprising administering a daily multivitamin, wherein the daily multivitamin comprises a daily recommended dose of essential vitamins and minerals.

Embodiment 383. The method of any one of embodiments 299-382, further comprising administering a therapeutically effective amount of an antihyperglycemia agent.

Embodiment 384. The method of embodiment 383, wherein the antihyperglycemia agent is metformin.

Embodiment 385. The method of embodiment 383, wherein the antihyperglycemia agent is acarbose.

Embodiment 386. The method of embodiment 383, wherein the antihyperglycemia agent is sodium-glucose cotransporter 2 inhibitor.

Embodiment 387. The method of embodiment 383, wherein the antihyperglycemia agent is DPP-4 inhibitor.

Embodiment 388. The method of embodiment 383, wherein the antihyperglycemia agent is a thiazolidinedione.

Embodiment 389. The method of embodiment 383, wherein the antihyperglycemia agent is a GLP-1 receptor agonist.

Embodiment 390. The method of embodiment 383, wherein the antihyperglycemia agent is an α-glucosidase inhibitor.

Embodiment 391. The method of embodiment 383, wherein the antihyperglycemia agent is a meglitinide.

Embodiment 392. The method of embodiment 383, wherein the antihyperglycemia agent is a sulfonylurea.

Embodiment 393. The method of embodiment 383, wherein the antihyperglycemia agent is insulin.

Embodiment 394. The method of any one of embodiments 299-393, further comprising administering a therapeutically effective amount of a second chemotherapeutic agent.

Embodiment 395. The method of embodiment 394, wherein the second chemotherapeutic agent is part of a standard-of-care therapy for the cancer.

Embodiment 396. The method of any one of embodiments 299-395, further comprising administering a radiotherapy.

77. A method of treating a cancer in a subject in need thereof, the method comprising administering to the subject:
a) an insulin suppressing meal;
b) about 100 to about 900 mg of an inhibitor of at least one kinase in the insulin receptor/PI3K/AKT/mTOR pathway; and
c) a therapeutically effective amount of a taxane-based chemotherapeutic agent, thereby treating the cancer in the subject.

78. The method of claim 77, wherein the cancer comprises a PIK3Cα mutation.

79. The method of claim 77, wherein the cancer comprises a PTEN loss-of-function mutation.

80. (canceled)

81. The method of claim 77, wherein the cancer is a solid tumor.

82. The method of claim 77, wherein the cancer is ovarian cancer.

83. The method of claim 77, wherein the cancer is endometrial cancer.

84. The method of claim 77, wherein the cancer is endometrial adenocarcinoma.

85. The method of claim 77, wherein the cancer is ovarian clear cell carcinoma.

86. The method of claim 77, wherein the cancer is ovarian endometrioid carcinoma.

87. The method of claim 77, wherein the cancer is colorectal cancer.

88. The method of claim 77, wherein the subject is on an insulin suppressing diet that comprises the insulin suppressing meal.

89. The method of claim 88, wherein the insulin suppressing diet comprises at least two insulin suppressing meals daily.

90.-93. (canceled)

94. The method of claim 88, wherein the insulin suppressing diet comprises a protein content of about 0.8-1.0 g/kg of a reference weight of the subject.

95. (canceled)

96. The method of claim 88, wherein the insulin suppressing diet comprises about 80-90% of total calories from fat.

97.-98. (canceled)

99. The method of claim 88, wherein the insulin suppressing diet comprises about 5-15% of total calories from protein.

100. (canceled)

101. The method of claim 88, wherein the insulin suppressing diet comprises about 1-10% of total calories from carbohydrate.

102.-103. (canceled)

104. The method of any one of claim 88, wherein the insulin suppressing diet comprises about 20-40 kcal/kg of a reference weight of the subject.

105. The method of claim 88, wherein the insulin suppressing diet comprises about 30-35 kcal/kg of a reference weight of the subject having a BMI<30 kg/m² or about 25-30 kcal/kg of a reference weight of the subject having a BMI>kg/m².

106.-114. (canceled)

115. The method of claim 77, wherein the administering of the inhibitor of the at least one kinase in the insulin receptor/PI3K/AKT/mTOR pathway is three consecutive days per week followed by four consecutive days without administering the inhibitor of the at least one kinase in the insulin receptor/PI3K/AKT/mTOR pathway.

116.-122. (canceled)

123. The method of claim 77, wherein the method comprises administering about 100 mg to about 600 mg of the inhibitor of the at least one kinase in the insulin receptor/PI3K/AKT/mTOR pathway.

124. (canceled)

125. The method of claim 77, wherein the inhibitor of the of the at least one kinase in the insulin receptor/PI3K/AKT/mTOR pathway is a PI3K inhibitor.

126. The method of claim 77, wherein the inhibitor of the of the at least one kinase in the insulin receptor/PI3K/AKT/mTOR pathway is a PI3Kα inhibitor.

127. The method of claim 77, wherein the inhibitor of the at least one kinase in the insulin receptor/PI3K/AKT/mTOR pathway is serabelisib, idelalisib, copanlisib, buparlisib (BKM120), alpelisib (BYL719), taselisib (GDC-0032), pictilisib (GDC-0941), apitolisib (GDC-0980), or dactolisib.

128.-135. (canceled)

136. The method of claim 77, wherein the inhibitor of the at least one kinase in the insulin receptor/PI3K/AKT/mTOR pathway is an AKT inhibitor.

137. The method of claim 77, wherein the inhibitor of the at least one kinase in the insulin receptor/PI3K/AKT/mTOR pathway is MK-2206.

138. The method of claim 77, wherein the inhibitor of the at least one kinase in the insulin receptor/PI3K/AKT/mTOR pathway is an inhibitor of the insulin receptor.

139. The method of claim 77, wherein the inhibitor of the at least one kinase in the insulin receptor/PI3K/AKT/mTOR pathway is linsitinib.

140. (canceled)

141. The method of claim 77, wherein the taxane-based chemotherapeutic agent is paclitaxel.

142.-143. (canceled)

144. The method of claim 77, wherein the therapeutically effective amount of the taxane-based chemotherapeutic agent is about 80 mg/m².

145.-147. (canceled)

148. The method of claim 77, wherein the administering of the taxane-based chemotherapeutic agent is intravenous.

149.-396. (canceled)
